(19) United States
(12) Patent Application Publication
(10) Pub. No.: US 2022/0396807 A1
(43) Pub. Date: Dec. 15, 2022

(54) METHODS FOR TREATING PATIENTS HAVING CFH MUTATIONS WITH CFH-ENCODING VECTORS

(71) Applicant: Gemini Therapeutics Sub, Inc., Cbridge, MA (US)

(72) Inventors: James McLaughlin, Cambridge, MA (US); Lisa Huang, Cambridge, MA (US); Suresh Katti, Hamden, CT (US); Walter Strapps, Dedham, MA (US); Melissa Deck, Acton, MA (US)

(21) Appl. No.: 17/771,336

(22) PCT Filed: Oct. 23, 2020
(86) PCT No.: PCT/US2020/057161

§ 371 Date: Apr. 22, 2022

Related U.S. Application Data
(60) Provisional application No. 62/925,073, filed on Oct. 23, 2019.

(51) Int. Cl. C12N 15/86; C07K 14/47; A61P 27/02

(52) U.S. Cl. CPC C12N 15/86; C07K 14/472; A61P 27/02; C12N 2750/14142; C12N 2750/14171; A61K 48/005

(57) ABSTRACT

The present disclosure provides methods for treating, preventing, or inhibiting diseases in patients having one or more mutations in complement factor H (CFH), complement component 3 (C3), and complement factor B (CFB) by administering to the patients a recombinant adeno-associated virus (rAAV) vector encoding a CFH polypeptide or biologically active fragment and/or variant thereof.

### CROSS-REFERENCE TO RELATED APPLICATIONS

This application claims the benefit of and priority to U.S. Provisional Patent Application No. 62/925,073, filed Oct. 23, 2019, the disclosures of which are incorporated by reference herein in their entireties for all purposes.

### SEQUENCE LISTING

The instant application contains a Sequence Listing which has been submitted electronically in ASCII format and is hereby incorporated by reference in its entirety. Said ASCII copy, created on Oct. 22, 2020, is named GEM-016WO_SL_ST25.txt and is 205,002 bytes in size.

### BACKGROUND OF THE DISCLOSURE

Age-related macular degeneration (AMD) is a medical condition and is the leading cause of legal blindness in Western societies. AMD typically affects older adults and results in a loss of central vision due to degenerative and neovascular changes to the macula, a pigmented region at the center of the retina which is responsible for visual acuity. There are four major AMD subtypes: Early AMD; Intermediate AMD; Advanced non-neovascular (“Dry”) AMD; and Advanced neovascular (“Wet”) AMD. Typically, AMD is identified by the focal hyperpigmentation of the retinal pigment epithelium (RPE) and accumulation of drusen deposits and/or geographic atrophy. The size and number of drusen deposits and level of geographic atrophy typically correlates with AMD severity. AMD occurs in up to 8% of individuals over the age of 60, and the prevalence of AMD continues to increase with age. The U.S. is anticipated to have nearly 22 million cases of AMD by the year 2050, while global cases of AMD are expected to be nearly 288 million by the year 2040.

There is a need for novel treatments for preventing progression from early to intermediate and/or from intermediate to advanced stages of AMD to prevent loss of vision, particularly in certain subpopulations of patients having one or more CFH mutations.

### SUMMARY OF THE DISCLOSURE

The disclosure provides methods of treating, preventing, or inhibiting diseases in a subject having a mutation in certain complement pathway genes by administering an effective amount of an AAV vector to express a complement factor H gene. The disease can be a disease of the eye, and the AAV vector can be delivered intraocularly (e.g., intravitreally) to the subject.

In one aspect, the present disclosure provides a method of treating a subject having a disease or disorder associated with undesired activity of the alternative complement pathway, comprising the step of administering to the subject an adeno-associated viral (AAV) vector comprising a nucleotide sequence encoding a Complement Factor H (CFH) polypeptide or biologically active fragment or variant thereof, and wherein the subject has one or more CFH, Complement Component 3 (C3), and/or Complement Factor B (CFB) gene mutations.

In another aspect, the present disclosure provides a method of treating a subject having age-related macular degeneration (AMD), comprising the step of administering to the subject an adeno-associated viral (AAV) vector comprising a nucleotide sequence encoding a Complement Factor H (CFH) polypeptide or biologically active fragment or variant thereof, and wherein the subject has one or more CFH, C3, and/or CFB gene mutations.

In certain embodiments, the amino acid sequence of the CFH polypeptide is at least 90% identical to the amino acid sequence of SEQ ID NO: 33 or a fragment thereof. In certain embodiments, the amino acid sequence of the CFH polypeptide is at least 95% identical to the amino acid sequence of SEQ ID NO: 33 or a fragment thereof. In certain embodiments, the amino acid sequence of the CFH polypeptide comprises the amino acid sequence of SEQ ID NO: 33 or a fragment thereof.

In certain embodiments, the nucleotide sequence is at least 90% identical to the nucleotide sequence of SEQ ID NO: 1, 2, 3 or 5, or codon-optimized variant and/or a fragment thereof. In certain embodiments, the nucleotide sequence is the sequence of SEQ ID NO: 1, 2, 3 or 5, or codon-optimized variant and/or a fragment thereof.

In certain embodiments, the CFH polypeptide or biologically active fragment or variant thereof comprises the V62 polymorphism. In certain embodiments, the CFH polypeptide or biologically active fragment or variant thereof comprises the Y402 polymorphism.

In certain embodiments, the CFH polypeptide or biologically active fragment or variant thereof comprises at least four CCP domains. In certain embodiments, the CFH polypeptide or biologically active fragment or variant thereof comprises at least five CCP domains. In certain embodiments, the CFH polypeptide or biologically active fragment or variant thereof comprises at least six CCP domains. In certain embodiments, the CFH polypeptide or biologically active fragment or variant thereof comprises at least seven CCP domains. In certain embodiments, the CFH polypeptide or biologically active fragment or variant thereof comprises at least three CCP domains.

In certain embodiments, the CFH polypeptide or biologically active fragment or variant thereof comprises the amino acid sequence of SEQ ID NO: 4. In certain embodiments, the amino acid sequence of SEQ ID NO: 4 is the C-terminal sequence of the CFH polypeptide or biologically active fragment or variant thereof.

In certain embodiments, the CFH polypeptide or biologically active fragment or variant thereof is capable of diffusing across the Bruch's membrane. In certain embodiments, the CFH polypeptide or biologically active fragment or variant thereof is capable of binding C3b. In certain embodiments, the CFH polypeptide or biologically active fragment or variant thereof is capable of facilitating the breakdown of C3b.

In certain embodiments, the vector comprises a promoter operably linked to the nucleotide sequence encoding the CFH polypeptide or biologically active fragment or variant thereof. In certain embodiments, the promoter is less than 1000 nucleotides in length. In certain embodiments, the promoter is less than 500 nucleotides in length. In certain embodiments, the promoter is less than 400 nucleotides in length.

In certain embodiments, the promoter comprises a CMV promoter. In certain embodiments, the promoter comprises the nucleotide sequence of SEQ ID NO: 8, or a fragment thereof. In certain embodiments, the promoter comprises the nucleotide sequence of SEQ ID NO: 12, or a fragment thereof. In certain embodiments, the promoter comprises the nucleotide sequence of SEQ ID NO: 14, or a fragment thereof. In certain embodiments, the promoter comprises the nucleotide sequence of SEQ ID NO: 31, or a fragment thereof. In certain embodiments, the promoter comprises the nucleotide sequence of SEQ ID NO: 36, or a fragment thereof.

In certain embodiments, the promoter is associated with strong expression in the eye. In certain embodiments, the promoter comprises a nucleotide sequence at least 90%, 95%, or 100% identical to the nucleotide sequence of any one of SEQ ID NOs: 6 or 32. In certain embodiments, the promoter comprises the nucleotide sequence of SEQ ID NO: 6, or a fragment thereof. In certain embodiments, the promoter comprises the nucleotide sequence of SEQ ID NO: 32, or a fragment thereof.

In certain embodiments, the promoter is associated with strong expression in the liver. In certain embodiments, the promoter comprises a nucleotide sequence at least 90%, 95% or 100% identical to the nucleotide sequence of any one of SEQ ID NOs: 16, 18, or 20. In certain embodiments, the promoter comprises the nucleotide sequence of SEQ ID NO: 16, or a fragment thereof. In certain embodiments, the promoter comprises the nucleotide sequence of SEQ ID NO: 18, or a fragment thereof. In certain embodiments, the promoter comprises the nucleotide sequence of SEQ ID NO: 20, or a fragment thereof.

In certain embodiments, the vector comprises a viral intron operably linked to the promoter. In certain embodiments, the viral intron comprises the nucleotide sequence of SEQ ID NO: 10, or a fragment thereof.

In certain embodiments, the vector comprises a Kozak sequence operably linked to the CFH polypeptide or biologically active fragment or variant thereof. In certain embodiments, the vector comprises a polyadenylation sequence operably linked to the CFH polypeptide or biologically active fragment or variant thereof.

In certain embodiments, the vector comprises AAV2 capsid proteins. In certain embodiments, the vector comprises AAV.7m8 capsid proteins. In certain embodiments, the vector comprises one or more ITR sequence flanking the vector portion encoding CFH. In certain embodiments, the vector comprises a selective marker. In certain embodiments, the selective marker is an antibiotic-resistance gene. In certain embodiments, the antibiotic-resistance gene is an ampicillin-resistance gene.

In certain embodiments, expression of the CFH polypeptide or biologically active fragment or variant thereof is induced in a target cell of the subject.

In certain embodiments, the target cell is a cell of the eye. In certain embodiments, the target cell is a cell of the retina. In certain embodiments, target cell is a cell of a layer of the retina selected from the group consisting of inner limiting membrane, nerve fiber, ganglion cell layer (GCL), inner plexiform layer, inner nuclear layer, outer plexiform layer, outer nuclear layer, external limiting membrane, rods and cones, and retinal pigment epithelium (RPE). In certain embodiments, the target cell is an RPE cell. In certain embodiments, the target cell is a cell of the macula. In certain embodiments, the vector is administered intraocularly. In certain embodiments, the vector is administered intravitreally. In certain embodiments, the vector is administered to the retina at a dose in the range of 1×10¹⁰ vg/eye to 1×10¹³ vg/eye. In certain embodiments, the vector or composition is administered to the retina at a dose of about 1.4×10¹² vg/eye. In certain embodiments, the level of the CFH polypeptide or biologically active fragment or variant thereof in the aqueous humor or vitreous humor of the subject is at least 50 ng/mL, at least 55 ng/mL, at least 60 ng/mL, at least 65 ng/mL, at least 70 ng/mL, at least 75 ng/mL, at least 80 ng/mL, at least 85 ng/mL, at least 90 ng/mL, or at least 100 ng/mL.

In certain embodiments, the target cell is a liver cell. In certain embodiments, the vector is administered systemically. In certain embodiments, the vector is administered intravenously. In certain embodiments, the level of the CFH polypeptide or biologically active fragment or variant thereof in the plasma of the subject is at least 100 μg/mL, at least 150 μg/mL, at least 200 μg/mL, at least 250 μg/mL, or at least 300 μg/mL.

In certain embodiments, the target cell is a cell of the choroid plexus.

In certain embodiments, the subject has a mutation in the CFH gene, optionally wherein the mutation is a loss-of-function mutation. In certain embodiments, the subject has one or more CFH mutations selected from the group consisting of: Y402H, R2T, L3V, R53C, R53H, S58A, D90G, D130N, R175Q, R175P, I221V, R303W, R303Q, Q400K, P503A, R567G, G650V, S890I, T956M, G1194D, or R1210C. In certain embodiments, the subject has any one or more of the following CFH mutations: R2T, R53C, R53H, S58A, D130N, R175Q, R175P, I221V, R303W, R303Q, P503A, R567G, G650V, G1194D, or R1210C. In certain embodiments, the subject has a Y402H mutation. In certain embodiments, the subject is homozygous for a Y402H mutation. In certain embodiments, the subject has an R2T mutation. In certain embodiments, the subject has an L3V mutation. In certain embodiments, the subject has an R53C mutation. In certain embodiments, the subject has an R53H mutation. In certain embodiments, the subject has an S58A mutation. In certain embodiments, the subject has a D90G mutation. In certain embodiments, the subject has a D130N mutation. In certain embodiments, the subject has an R175Q mutation. In certain embodiments, the subject has an R175P mutation. In certain embodiments, the subject has an I221V mutation. In certain embodiments, the subject has an R303W mutation. In certain embodiments, the subject has an R303Q mutation. In certain embodiments, the subject has a Q400K mutation. In certain embodiments, the subject has a P503A mutation. In certain embodiments, the subject has an R567G mutation. In certain embodiments, the subject has a G650V mutation. In certain embodiments, the subject has an S890I mutation. In certain embodiments, the subject has a T956M mutation. In certain embodiments, the subject has a G1194D mutation. In certain embodiments, the subject has an R1210C mutation.

In certain embodiments, the one or more CFH mutations reduce CFH activity as compared to a wildtype CFH polypeptide. In certain embodiments, the CFH activity is the ability to bind to C3b. In certain embodiments, the CFH activity has the ability to act as a cofactor with CFI and facilitate C3b cleavage. In certain embodiments, the CFH activity is the ability to bind to a cell surface. In certain embodiments, the CFH activity is the ability to bind to heparin. In certain embodiments, the CFH activity is the ability to reduce C5b9 levels generated as a result of complement activation. In certain embodiments, the CFH activity is the ability to inhibit hemolysis. In certain embodiments, the wildtype CFH polypeptide comprises a CFH polypeptide having the amino acid sequence of any one of SEQ ID NOs: 33, 34, 37 or 38.

In certain embodiments, the subject has a mutation in the subject's C3 gene, optionally wherein the mutation is a gain-of-function mutation. In certain embodiments, the subject has one or more of the following C3 mutations: R102G, K155Q, V619M, and R735W.

In certain embodiments, the subject has a mutation in the subject's CFB gene, optionally wherein the mutation is a gain-of-function mutation. In certain embodiments, the subject has the I242L mutation of CFB.

In certain embodiments, the subject is homozygous for CFH 62V, C3 102G, and complement factor B (CFB) 32R.

In certain embodiments, the subject is homozygous for at least one of the one or more CFH, C3, and/or CFB mutations. In certain embodiments, the subject is heterozygous for at least one of the one or more CFH, C3, and/or CFB mutations.

In certain embodiments, the subject has been determined to have the one or more CFH, C3, and/or CFB mutations.

In certain embodiments, the subject has atypical hemolytic uremic syndrome (aHUS). In certain embodiments, the subject has a renal disease or complication.

### BRIEF DESCRIPTION OF THE DRAWINGS

FIG. 1A shows a simplified schematic of the CFH protein bound to C3b. CCPs 1-20 are shown, along with the site of the R53C mutation. FIG. 1B is a graph showing CFH binding ratio to C3b protein. FIG. 1C is a graph plotting cofactor activity of CFH protein. FIG. 1D is a graph showing results from a decay acceleration assay. FIG. 1E is a graph showing results from a Weislab activity assay. FIG. 1F is a graph showing results from a hemolysis inhibition assay. In each assay, VYE control CFH was tested as compared to the R53C mutant CFH protein. The VYE control CFH used in these and other experiments described herein included three prevalent polymorphisms: V62, Y402 and E936. As shown in FIGS. 1B-1F, the R53C mutant CFH has impaired function.

FIG. 2A shows a simplified schematic of the CFH protein bound to C3b. CCPs 1-20 are shown, along with the site of the R53H mutation. FIG. 2B is a graph showing CFH binding ratio to C3b protein. FIG. 2C is a graph plotting cofactor activity of CFH protein. FIG. 2D is a graph showing results from a decay acceleration assay. FIG. 2E is a graph showing results from a Weislab activity assay. FIG. 2F is a graph showing results from a hemolysis inhibition assay. In each assay, VYE control CFH was tested as compared to the R53H mutant CFH protein. As shown in FIGS. 2C-2F, the R53H mutant CFH has impaired function.

FIG. 3A shows a simplified schematic of the CFH protein bound to C3b. CCPs 1-20 are shown, along with the site of the S58A mutation. FIG. 3B is a graph showing CFH binding ratio to C3b protein. FIG. 3C is a graph plotting cofactor activity of CFH protein. FIG. 3D is a graph showing results from a decay acceleration assay. FIG. 3E is a graph showing results from a Weislab activity assay. FIG. 3F is a graph showing results from a hemolysis inhibition assay. In each assay, VYE control CFH was tested as compared to the S58A mutant CFH protein. As shown in FIG. 3C, the S58A mutant CFH has impaired function.

FIG. 4A shows a simplified schematic of the CFH protein bound to C3b. CCPs 1-20 are shown, along with the site of the D90G mutation. FIG. 4B is a graph showing CFH binding ratio to C3b protein. FIG. 4C is a graph plotting cofactor activity of CFH protein. FIG. 4D is a graph showing results from a decay acceleration assay. FIG. 4E is a graph showing results from a Weislab activity assay. FIG. 4F is a graph showing results from a hemolysis inhibition assay. In each assay, VYE control CFH was tested as compared to the D90G mutant CFH protein.

FIG. 5A shows a simplified schematic of the CFH protein bound to C3b. CCPs 1-20 are shown, along with the site of the D130N mutation. FIG. 5B is a graph showing CFH binding ratio to C3b protein. FIG. 5C is a graph plotting cofactor activity of CFH protein. FIG. 5D is a graph showing results from a decay acceleration assay. FIG. 5E is a graph showing results from a Weislab activity assay. FIG. 5F is a graph showing results from a hemolysis inhibition assay. In each assay, VYE control CFH was tested as compared to the D130N mutant CFH protein. As shown in FIGS. 5C, 5D and potentially FIGS. 5E and 5F, the D130N mutant CFH has impaired function.

FIG. 6A shows a simplified schematic of the CFH protein bound to C3b. CCPs 1-20 are shown, along with the site of the R175Q mutation. FIG. 6B is a graph showing CFH binding ratio to C3b protein. FIG. 6C is a graph plotting cofactor activity of CFH protein. FIG. 6D is a graph showing results from a decay acceleration assay. FIG. 6E is a graph showing results from a Weislab activity assay. FIG. 6F is a graph showing results from a hemolysis inhibition assay. In each assay, VYE control CFH was tested as compared to the R175Q mutant CFH protein. As shown in FIG. 6B-6E, the R175Q mutant CFH has impaired function.

FIG. 7A shows a simplified schematic of the CFH protein bound to C3b. CCPs 1-20 are shown, along with the site of the R175P mutation. FIG. 7B is a graph showing CFH binding ratio to C3b protein. FIG. 7C is a graph plotting cofactor activity of CFH protein. FIG. 7D is a graph showing results from a decay acceleration assay. FIG. 7E is a graph showing results from a Weislab activity assay. FIG. 7F is a graph showing results from a hemolysis inhibition assay. In each assay, VYE control CFH was tested as compared to the R175P mutant CFH protein. As shown in FIGS. 7B-7F, the R175P mutant CFH has impaired function.

FIG. 8A shows a simplified schematic of the CFH protein bound to C3b. CCPs 1-20 are shown, along with the site of the I221V mutation. FIG. 8B is a graph showing CFH binding ratio to C3b protein. FIG. 8C is a graph plotting cofactor activity of CFH protein. FIG. 8D is a graph showing results from a decay acceleration assay. FIG. 8E is a graph showing results from a Weislab activity assay. FIG. 8F is a graph showing results from a hemolysis inhibition assay. In each assay, VYE control CFH was tested as compared to the I221V mutant CFH protein. As shown in FIG. 8C and potentially FIG. 8E, the I221V mutant CFH has impaired function.

FIG. 9A shows a simplified schematic of the CFH protein bound to C3b. CCPs 1-20 are shown, along with the site of the R303W mutation. FIG. 9B is a graph showing CFH binding ratio to C3b protein. FIG. 9C is a graph plotting cofactor activity of CFH protein. FIG. 9D is a graph showing results from a decay acceleration assay. FIG. 9E is a graph showing results from a Weislab activity assay. FIG. 9F is a graph showing results from a hemolysis inhibition assay. In each assay, VYE control CFH was tested as compared to the R303W mutant CFH protein. As shown in FIG. 9F and potentially FIGS. 9C and 9E, the R303W mutant CFH has impaired function.

FIG. 10A shows a simplified schematic of the CFH protein bound to C3b. CCPs 1-20 are shown, along with the site of the R303Q mutation. FIG. 10B is a graph showing CFH binding ratio to C3b protein. FIG. 10C is a graph plotting cofactor activity of CFH protein. FIG. 10D is a graph showing results from a decay acceleration assay. FIG. 10E is a graph showing results from a Weislab activity assay. FIG. 10F is a graph showing results from a hemolysis inhibition assay. In each assay, VYE control CFH was tested as compared to the R303Q mutant CFH protein. As shown in FIGS. 10B and 10F and potentially 10C-10E, the R303Q mutant CFH has impaired function.

FIG. 11A shows a simplified schematic of the CFH protein bound to C3b. CCPs 1-20 are shown, along with the site of the Q400K mutation. FIG. 11B is a graph showing CFH binding ratio to C3b protein. FIG. 11C is a graph plotting cofactor activity of CFH protein. FIG. 11D is a graph showing results from a decay acceleration assay. FIG. 11E is a graph showing results from a Weislab activity assay. FIG. 11F is a graph showing results from a hemolysis inhibition assay. In each assay, VYE control CFH was tested as compared to the Q400K mutant CFH protein.

FIG. 12A shows a simplified schematic of the CFH protein bound to C3b. CCPs 1-20 are shown, along with the site of the Y402H mutation. FIG. 12B is a graph showing CFH binding ratio to C3b protein. FIG. 12C is a graph plotting cofactor activity of CFH protein. FIG. 12D is a graph showing results from a decay acceleration assay. FIG. 12E is a graph showing results from a Weislab activity assay. FIG. 12F is a graph showing results from a hemolysis inhibition assay. In each assay, VYE control CFH was tested as compared to the Y402H mutant CFH protein.

FIG. 13A shows a simplified schematic of the CFH protein bound to C3b. CCPs 1-20 are shown, along with the site of the P503A mutation. FIG. 13B is a graph showing CFH binding ratio to C3b protein. FIG. 13C is a graph plotting cofactor activity of CFH protein. FIG. 13D is a graph showing results from a decay acceleration assay. FIG. 13E is a graph showing results from a Weislab activity assay. FIG. 13F is a graph showing results from a hemolysis inhibition assay. In each assay, VYE control CFH was tested as compared to the P503A mutant CFH protein. As shown in FIGS. 13B and 13E-13F and potentially FIG. 13D, the P503A mutant CFH has impaired function.

FIG. 14A shows a simplified schematic of the CFH protein bound to C3b. CCPs 1-20 are shown, along with the site of the R567G mutation. FIG. 14B is a graph showing CFH binding ratio to C3b protein. FIG. 14C is a graph plotting cofactor activity of CFH protein. FIG. 14D is a graph showing results from a decay acceleration assay. FIG. 14E is a graph showing results from a Weislab activity assay. FIG. 14F is a graph showing results from a hemolysis inhibition assay. In each assay, VYE control CFH was tested as compared to the R567G mutant CFH protein. As shown in FIGS. 14B and 14D-14F and potentially 14C, the R567G mutant CFH has impaired function.

FIG. 15A shows a simplified schematic of the CFH protein bound to C3b. CCPs 1-20 are shown, along with the site of the G650V mutation. FIG. 15B is a graph showing CFH binding ratio to C3b protein. FIG. 15C is a graph plotting cofactor activity of CFH protein. FIG. 15D is a graph showing results from a decay acceleration assay. FIG. 15E is a graph showing results from a Weislab activity assay. FIG. 15F is a graph showing results from a hemolysis inhibition assay. In each assay, VYE control CFH was tested as compared to the G650V mutant CFH protein. As shown in FIG. 15C, the G650V mutant CFH has impaired function.

FIG. 16A shows a simplified schematic of the CFH protein bound to C3b. CCPs 1-20 are shown, along with the site of the S890I mutation. FIG. 16B is a graph showing CFH binding ratio to C3b protein. FIG. 16C is a graph plotting cofactor activity of CFH protein. FIG. 16D is a graph showing results from a decay acceleration assay. FIG. 16E is a graph showing results from a Weislab activity assay. FIG. 16F is a graph showing results from a hemolysis inhibition assay. In each assay, VYE control CFH was tested as compared to the S890I mutant CFH protein.

FIG. 17A shows a simplified schematic of the CFH protein bound to C3b. CCPs 1-20 are shown, along with the site of the T956M mutation. FIG. 17B is a graph showing CFH binding ratio to C3b protein. FIG. 17C is a graph plotting cofactor activity of CFH protein. FIG. 17D is a graph showing results from a decay acceleration assay. FIG. 17E is a graph showing results from a Weislab activity assay. FIG. 17F is a graph showing results from a hemolysis inhibition assay. In each assay, VYE control CFH was tested as compared to the T956M mutant CFH protein.

FIG. 18A shows a simplified schematic of the CFH protein bound to C3b. CCPs 1-20 are shown, along with the site of the G1194D mutation. FIG. 18B is a graph showing CFH binding ratio to C3b protein. FIG. 18C is a graph plotting cofactor activity of CFH protein. FIG. 18D is a graph showing results from a decay acceleration assay. FIG. 18E is a graph showing results from a Weislab activity assay. FIG. 18F is a graph showing results from a hemolysis inhibition assay. In each assay, VYE control CFH was tested as compared to the G1194D mutant CFH protein. As shown in FIG. 18C-18F, the G1194D mutant CFH has impaired function.

FIG. 19A shows a simplified schematic of the CFH protein bound to C3b. CCPs 1-20 are shown, along with the site of the R1210C mutation. FIG. 19B is a graph showing CFH binding ratio to C3b protein. FIG. 19C is a graph plotting cofactor activity of CFH protein. FIG. 19D is a graph showing results from a decay acceleration assay. FIG. 19E is a graph showing results from a Weislab activity assay. FIG. 19F is a graph showing results from a hemolysis inhibition assay. In each assay, VYE control CFH was tested as compared to the R1210C mutant CFH protein. As shown in FIGS. 19B-19D and potentially 19E and 19F, the R1210C mutant CFH has impaired function.

FIG. 20A shows a simplified schematic of the CFH protein bound to C3b. CCPs 1-20 are shown. FIG. 20B is a graph showing CFH binding ratio to C3b protein. FIG. 20C is a graph plotting cofactor activity of CFH protein. FIG. 20D is a graph showing results from a decay acceleration assay. FIG. 20E is a graph showing results from a Weislab activity assay. FIG. 20F is a graph showing results from a hemolysis inhibition assay. In each assay, VYE control CFH was tested as compared to the R2T mutant CFH protein.

FIG. 21A shows a simplified schematic of the CFH protein bound to C3b. CCPs 1-20 are shown. FIG. 21B is a graph showing CFH binding ratio to C3b protein. FIG. 21C is a graph plotting cofactor activity of CFH protein. FIG. 21D is a graph showing results from a decay acceleration assay. FIG. 21E is a graph showing results from a Weislab activity assay. FIG. 21F is a graph showing results from a hemolysis inhibition assay. In each assay, VYE control CFH was tested as compared to the L3V mutant CFH protein.

FIG. 22 shows a bar graph summarizing the C3b binding assay results from FIGS. 1B, 2B, 3B, 4B, 5B, 6B, 7B, 8B, 9B, 10B, 11B, 12B, 13B, 14B, 15B, 16B, 17B, 18B, 19B, 20B, and 21B based on ratios as compared to the VYE CFH control values. CFH mutants having ratios<0.9 were identified as being functionally deficient in the C3b binding assay. The dotted line corresponds to the 0.9 ratio. Bars above that line are indicated in bars with dots, while bars below that line are indicated in bars with stripes. The black bar corresponds to the VYE control.

FIG. 23 shows a bar graph summarizing the cofactor activity assay results from FIGS. 1C, 2C, 3C, 4C, 5C, 6C, 7C, 8C, 9C, 10C, 11C, 12C, 13C, 14C, 15C, 16C, 17C, 18C, 19C, 20C, and 21C based on ratios as compared to the VYE CFH control values. CFH mutants having ratios>1.1 were identified as being functionally deficient in the cofactor activity assay. The dotted line corresponds to the 1.1 ratio. Bars above the dashed line are indicated in bars with stripes, while bars below that line are indicated in bars with dots. The black bar corresponds to the VYE control.

FIG. 24 shows a bar graph summarizing the decay acceleration assay results from FIGS. 1D, 2D, 3D, 4D, 5D, 6D, 7D, 8D, 9D, 10D, 11D, 12D, 13D, 14D, 15D, 16D, 17D, 18D, 19D, 20D, and 21D based on ratios as compared to the VYE CFH control values. CFH mutants having ratios<0.9 were identified as being functionally deficient in the decay acceleration assay. The dotted line corresponds to the 0.9 ratio. Bars above that line are indicated in bars with dots, while bars below that line are indicated in bars with stripes. The black bar corresponds to the VYE control.

FIG. 25 shows a bar graph summarizing the Weislab assay results from FIGS. 1E, 2E, 3E, 4E, 5E, 6E, 7E, 8E, 9E, 10E, 11E, 12E, 13E, 14E, 15E, 16E, 17E, 18E, 19E, 20E, and 21E based on ratios as compared to the VYE CFH control values. CFH mutants having ratios>1.6 were identified as being functionally deficient in the Weislab® assay. The dotted line corresponds to the 1.6 ratio. Bars above that line are indicated in bars with stripes, while bars below that line are indicated in bars with dots. The black bar corresponds to the VYE control.

FIG. 26 shows a bar graph comparing the cell expression of VYE control CFH as compared to each of the respective CFH mutants. CFH mutants having ratios<0.9 were identified as being associated with functionally deficient with regard to cell expression. The dotted line corresponds to the 0.9 ratio. Bars above that line are indicated in bars with stripes, while bars below that line are indicated in bars with dots. The black bar corresponds to the VYE control.

FIG. 27 is a table indicating whether each CFH mutant was identified as being functionally deficient in each of the recited assays: the cell expression assay, the C3b binding assay, the decay acceleration assay, the cofactor activity assay, the decay acceleration assay, the Weislab® assay, and the hemolysis assay. “Y” means functionally deficient in the respective assay, “N” means not functionally deficient in the respective assay, and “M” means maybe functionally deficient in the respective assay.

FIG. 28 shows a vector map of a full vector genome construct for expression of CFH. “ITR” corresponds to inverted terminal repeats; “CRALBP promoter” corresponds to the cellular retinaldehyde-binding protein promoter; “CFH” corresponds to the gene encoding Complement Factor H; “polyA” corresponds to the polyadenylation sequence; “Amp R” corresponds to the ampicillin resistance cassette. The nucleotide sequence corresponding to the vector illustrated in FIG. 28 is SEQ ID NO: 7.

FIG. 29 shows a vector map of a full vector genome construct for expression of CFH. “ITR” corresponds to inverted terminal repeats; “EFla promoter” corresponds to the elongation factor-1 alpha promoter; “CFH” corresponds to the gene encoding Complement Factor H; “polyA” corresponds to the polyadenylation sequence; “AmpR” corresponds to the ampicillin resistance cassette. The nucleotide sequence corresponding to the vector illustrated in FIG. 29 is SEQ ID NO: 9.

FIG. 30 shows a vector map of a full vector genome construct for expression of CFH. “ITR” corresponds to inverted terminal repeats; “EF1a.SV40i” corresponds to the elongation factor-1 alpha promoter including the simian virus 40 intron; “CFH” corresponds to the gene encoding Complement Factor H; “polyA” corresponds to the polyadenylation sequence; “AmpR” corresponds to the ampicillin resistance cassette. The nucleotide sequence corresponding to the vector illustrated in FIG. 30 is SEQ ID NO: 11.

FIG. 31 shows a vector map of a full vector genome construct for expression of CFH. “ITR” corresponds to inverted terminal repeats; “HSP70 promoter” corresponds to the heat shock protein 70 promoter; “CFH” corresponds to the gene encoding Complement Factor H; “polyA” corresponds to the polyadenylation sequence; “AmpR” corresponds to the ampicillin resistance cassette. The nucleotide sequence corresponding to the vector illustrated in FIG. 31 is SEQ ID NO: 13.

FIG. 32 shows a vector map of a full vector genome construct for expression of CFH. “ITR” corresponds to inverted terminal repeats; “sCBA promoter” corresponds to the chicken β actin promoter; “CFH” corresponds to the gene encoding Complement Factor H; “polyA” corresponds to the polyadenylation sequence; “AmpR” corresponds to the ampicillin resistance cassette. This vector also included the SV40i intron. The nucleotide sequence corresponding to the vector illustrated in FIG. 32 is SEQ ID NO: 15.

FIG. 33 shows a vector map of a full vector genome construct for expression of CFH. “ITR” corresponds to inverted terminal repeats; “AAT1” corresponds to the alpha-1 antitrypsin 1 promoter; “CFH” corresponds to the gene encoding Complement Factor H; “polyA” corresponds to the polyadenylation sequence; “AmpR” corresponds to the ampicillin resistance cassette. The nucleotide sequence corresponding to the vector illustrated in FIG. 33 is SEQ ID NO: 17.

FIG. 34 shows a vector map of a full vector genome construct for expression of CFH. “ITR” corresponds to inverted terminal repeats; “ALB” corresponds to a synthetic promoter based on the human albumin promoter; “CFH” corresponds to the gene encoding Complement Factor H; “polyA” corresponds to the polyadenylation sequence; “AmpR” corresponds to the ampicillin resistance cassette. The nucleotide sequence corresponding to the vector illustrated in FIG. 34 is SEQ ID NO: 19.

FIG. 35 shows a vector map of a full vector genome construct for expression of CFH. “ITR” corresponds to inverted terminal repeats; “PCK1” corresponds to the phosphoenolpyruvate carboxykinase 1 promoter; “CFH” corresponds to the gene encoding Complement Factor H; “polyA” corresponds to the polyadenylation sequence; “AmpR” corresponds to the ampicillin resistance cassette. The nucleotide sequence corresponding to the vector illustrated in FIG. 35 is SEQ ID NO: 21.

FIG. 36 shows a vector map of a full vector genome construct for expression of FHL-1. “ITR” corresponds to inverted terminal repeats; “EF1a” corresponds to the elongation factor-1 alpha promoter; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 36 is SEQ ID NO: 22.

FIG. 37 shows a vector map of a full vector genome construct for expression of FHL-1. “ITR” corresponds to inverted terminal repeats; “ALB” corresponds to a synthetic promoter based on the human albumin promoter; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 37 is SEQ ID NO: 23.

FIG. 38 shows a vector map of a full vector genome construct for expression of FHL-1. “ITR” corresponds to inverted terminal repeats; “AAT1” corresponds to the alpha-1 antitrypsin 1 promoter; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 38 is SEQ ID NO: 24.

FIG. 39 shows a vector map of a full vector genome construct for expression of FHL-1. “ITR” corresponds to inverted terminal repeats; “EF1a.SV40i” corresponds to the elongation factor-1 alpha promoter including the simian virus 40 intron; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 39 is SEQ ID NO: 25.

FIG. 40 shows a vector map of a full vector genome construct for expression of FHL-1. “ITR” corresponds to inverted terminal repeats; “CAG” corresponds to a synthetic promoter that includes the cytomegalovirus (CMV) early enhancer element, the promoter/first exon/first intron of chicken beta-actin gene, and the splice acceptor of the rabbit beta-globin gene; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 40 is SEQ ID NO: 26.

FIG. 41 shows a vector map of a full vector genome construct for expression of FHL-1. “ITR” corresponds to inverted terminal repeats; “CRALBP” corresponds to the cellular retinaldehyde-binding protein promoter; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 41 is SEQ ID NO: 27.

FIG. 42 shows a vector map of a full vector genome construct for expression of FHL-1. “ITR” corresponds to inverted terminal repeats; “hRPE65” corresponds to the retinal pigment epithelial 65 promoter; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 42 is SEQ ID NO: 28.

FIG. 43 shows a vector map of a full vector genome construct for expression of FHL-1. “ITR” corresponds to inverted terminal repeats; “HSP70” corresponds to the heat shock protein 70 promoter; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 43 is SEQ ID NO: 29.

FIG. 44 shows a vector map of a full vector genome construct for expression of FHL-1. “ITR” corresponds to inverted terminal repeats; “PCK1” corresponds to the phosphoenolpyruvate carboxykinase 1 promoter; “FHL-1” corresponds to the gene encoding Factor-H-Like Protein 1; “polyA” corresponds to the polyadenylation sequence; “AmpR” corresponds to the ampicillin resistance cassette. The nucleotide sequence corresponding to the vector illustrated in FIG. 44 is SEQ ID NO: 30.

### DETAILED DESCRIPTION OF THE DISCLOSURE

The disclosure provides methods of treating, preventing, or inhibiting diseases in a subject having a mutation in certain complement pathway genes by administering an effective amount of an AAV vector to express a complement factor H gene. The disease can be a disease of the eye, and the AAV vector can be delivered intraocularly (e.g., intravitreally) to the subject.

In one aspect, the present disclosure provides a method of treating a subject having a disease or disorder associated with undesired activity of the alternative complement pathway, comprising the step of administering to the subject an adeno-associated viral (AAV) vector comprising a nucleotide sequence encoding a Complement Factor H (CFH) polypeptide or biologically active fragment or variant thereof, and wherein the subject has one or more CFH, Complement Component 3 (C3), and/or Complement Factor B (CFB) gene mutations. In another aspect, the present disclosure provides a method of treating a subject having a disease or disorder associated with undesired activity of the alternative complement pathway, the method comprising the step of (a) selecting a subject that has one or more CFH, C3, and/or CFB gene mutations; and (b) administering to the subject an adeno-associated viral (AAV) vector comprising a nucleotide sequence encoding a Complement Factor H (CFH) polypeptide or biologically active fragment or variant thereof.

In another aspect, the present disclosure provides a method of treating a subject having age-related macular degeneration (AMD), comprising the step of administering to the subject an adeno-associated viral (AAV) vector comprising a nucleotide sequence encoding a Complement Factor H (CFH) polypeptide or biologically active fragment or variant thereof, and wherein the subject has one or more CFH, C3, and/or CFB gene mutations. In another aspect, the present disclosure provides a method of treating a subject having age-related macular degeneration (AMD), the method comprising the step of (a) selecting a subject that has one or more CFH, C3, and/or CFB gene mutations; and (b) administering to the subject an adeno-associated viral (AAV) vector comprising a nucleotide sequence encoding a Complement Factor H (CFH) polypeptide or biologically active fragment or variant thereof.

A wide variety of diseases of the eye may be treated or prevented using the viral vectors and methods provided herein. Diseases of the eye that may be treated or prevented using the vectors and methods of the disclosure include but are not limited to, glaucoma, macular degeneration (e.g., age-related macular degeneration), diabetic retinopathies, inherited retinal degeneration such as retinitis pigmentosa, retinal detachment or injury and retinopathies (such as retinopathies that are inherited, induced by surgery, trauma, an underlying aetiology such as severe anemia, SLE, hypertension, blood dyscrasias, systemic infections, or underlying carotid disease, a toxic compound or agent, or photically).

### General Techniques

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons, N Y (2003); Short Protocols in Molecular Biology (Wiley and Sons, 1999).

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well-known and commonly used in the art. Standard techniques are used for chemical syntheses, and chemical analyses.

Throughout this specification and embodiments, the word “comprise,” or variations such as “comprises” or “comprising,” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the embodied disclosure.

Exemplary methods and materials are described herein, although methods and materials similar or equivalent to those described herein can also be used in the practice or testing of the present disclosure. The materials, methods, and examples are illustrative only and not intended to be limiting.

### Definitions

The following terms, unless otherwise indicated, shall be understood to have the following meanings:

As used herein, “residue” refers to a position in a protein and its associated amino acid identity.

As used herein, the term “CFH” or “Complement Factor H” encompasses complement factor H and factor-H-like protein 1 (FHL1). Exemplary amino acid sequences of CFH are SEQ ID NOs: 33-34 and 37-38, with SEQ ID NO: 34 corresponding to the FHL1 amino acid sequence.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The terms “polypeptide,” “oligopeptide,” “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions.

However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” or “percent (%) identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, a “host cell” includes an individual cell or cell culture that can be or has been a recipient for vector(s) for incorporation of polynucleotide inserts. The term host cell may refer to the packaging cell line in which the rAAV is produced from the plasmid.

In the alternative, the term “host cell” may refer to the target cell in which expression of the transgene is desired.

As used herein, a “vector,” refers to a recombinant plasmid or virus that comprises a nucleic acid to be delivered into a host cell, either in vitro or in vivo. A “recombinant viral vector” refers to a recombinant polynucleotide vector comprising one or more heterologous sequences (i.e. a nucleic acid sequence not of viral origin). In the case of recombinant AAV vectors, the recombinant nucleic acid is flanked by at least one inverted terminal repeat sequence (ITR). In some embodiments, the recombinant nucleic acid is flanked by two ITRs.

A “recombinant AAV vector (rAAV vector)” refers to a polynucleotide vector based on an adeno-associated virus comprising one or more heterologous sequences (i.e., nucleic acid sequence not of AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) and that is expressing AAV rep and cap gene products (i.e., AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. An rAAV vector can be in any of a number of forms, including, but not limited to, plasmids, linear artificial chromosomes, complexed with lipids, encapsulated within liposomes, and encapsidated in a viral particle, e.g., an AAV particle. An rAAV vector can be packaged into an AAV virus capsid to generate a “recombinant adeno-associated viral particle (rAAV particle).”

An “rAAV virus” or “rAAV viral particle” refers to a viral particle composed of at least one AAV capsid protein and an encapsidated rAAV vector genome.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome.

The term “vector genome (vg)” as used herein may refer to one or more polynucleotides comprising a set of the polynucleotide sequences of a vector, e.g., a viral vector. A vector genome may be encapsidated in a viral particle. Depending on the particular viral vector, a vector genome may comprise single-stranded DNA, double-stranded DNA, or single-stranded RNA, or double-stranded RNA. A vector genome may include endogenous sequences associated with a particular viral vector and/or any heterologous sequences inserted into a particular viral vector through recombinant techniques. For example, a recombinant AAV vector genome may include at least one ITR sequence flanking a promoter, a stuffer, a sequence of interest (e.g., an RNAi), and a polyadenylation sequence. A complete vector genome may include a complete set of the polynucleotide sequences of a vector. In some embodiments, the nucleic acid titer of a viral vector may be measured in terms of vg/mL. Methods suitable for measuring this titer are known in the art (e.g., quantitative PCR).

An “inverted terminal repeat” or “ITR” sequence is a term well understood in the art and refers to relatively short sequences found at the termini of viral genomes which are in opposite orientation.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C and D regions), allowing intrastrand base-pairing to occur within this portion of the ITR.

A “helper virus” for AAV refers to a virus that allows AAV (which is a defective parvovirus) to be replicated and packaged by a host cell. A number of such helper viruses are known in the art.

As used herein, “expression control sequence” means a nucleic acid sequence that directs transcription of a nucleic acid. An expression control sequence can be a promoter, such as a constitutive promoter, or an enhancer. The expression control sequence is operably linked to the nucleic acid sequence to be transcribed.

As used herein, “isolated molecule” (where the molecule is, for example, a polypeptide, a polynucleotide, or fragment thereof) is a molecule that by virtue of its origin or source of derivation (1) is not associated with one or more naturally associated components that accompany it in its native state, (2) is substantially free of one or more other molecules from the same species (3) is expressed by a cell from a different species, or (4) does not occur in nature.

As used herein, “purify,” and grammatical variations thereof, refers to the removal, whether completely or partially, of at least one impurity from a mixture containing the polypeptide and one or more impurities, which thereby improves the level of purity of the polypeptide in the composition (i.e., by decreasing the amount (ppm) of impurity(ies) in the composition).

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

The terms “patient,” “subject,” or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In some embodiments, the subject is a human that is at least 40, at least 45, at least 50, at least 55, at least 60, at least 65, at least 70, at least 75, at least 80, at least 85, at least 90, or at least 95 years of age.

In one embodiment, the subject has, or is at risk of developing a disease of the eye. A disease of the eye, includes, without limitation, retinitis pigmentosa, rod-cone dystrophy, Leber's congenital amaurosis, Usher's syndrome, Bardet-Biedl Syndrome, Best disease, retinoschisis, Stargardt disease (autosomal dominant or autosomal recessive), untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, glaucoma, or retinal vein occlusion. In another embodiment, the subject has, or is at risk of developing glaucoma, Leber's hereditary optic neuropathy, lysosomal storage disorder, or peroxisomal disorder. In another embodiment, the subject is in need of optogenetic therapy. In another embodiment, the subject has shown clinical signs of a disease of the eye.

In some embodiments, the subject has, or is at risk of developing a renal disease or complication. In some embodiments, the renal disease or complication is associated with AMD or aHUS.

In some embodiments, the subject has, or is at risk of developing AMD or aHUS.

Clinical signs of a disease of the eye include, but are not limited to, decreased peripheral vision, decreased central (reading) vision, decreased night vision, loss of color perception, reduction in visual acuity, decreased photoreceptor function, and pigmentary changes. In one embodiment, the subject shows degeneration of the outer nuclear layer (ONL). In another embodiment, the subject has been diagnosed with a disease of the eye. In yet another embodiment, the subject has not yet shown clinical signs of a disease of the eye.

As used herein, the terms “prevent,” “preventing,” and “prevention” refer to the prevention of the recurrence or onset of, or a reduction in one or more symptoms of a disease or condition (e.g., a disease of the eye) in a subject as result of the administration of a therapy (e.g., a prophylactic or therapeutic agent). For example, in the context of the administration of a therapy to a subject for an infection, “prevent,” “preventing,” and “prevention” refer to the inhibition or a reduction in the development or onset of a disease or condition (e.g., a disease of the eye), or the prevention of the recurrence, onset, or development of one or more symptoms of a disease or condition (e.g., a disease of the eye), in a subject resulting from the administration of a therapy (e.g., a prophylactic or therapeutic agent), or the administration of a combination of therapies (e.g., a combination of prophylactic or therapeutic agents).

“Treating” a condition or patient refers to taking steps to obtain beneficial or desired results, including clinical results. With respect to a disease or condition (e.g., a disease of the eye), treatment refers to the reduction or amelioration of the progression, severity, and/or duration of an infection (e.g., a disease of the eye or symptoms associated therewith), or the amelioration of one or more symptoms resulting from the administration of one or more therapies (including, but not limited to, the administration of one or more prophylactic or therapeutic agents).

“Administering” or “administration of” a substance, a compound or an agent to a subject can be carried out using one of a variety of methods known to those skilled in the art. For example, a compound or an agent can be administered intravitreally or subretinally. In particular embodiments, the compound or agent is administered intravitreally. In some embodiments, administration may be local. In other embodiments, administration may be systemic. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

As used herein, the term “ocular cells” refers to any cell in, or associated with the function of, the eye. The term may refer to any one or more of photoreceptor cells, including rod, cone and photosensitive ganglion cells, retinal pigment epithelium (RPE) cells, glial cells, Muller cells, bipolar cells, horizontal cells, amacrine cells. In one embodiment, the ocular cells are bipolar cells. In another embodiment, the ocular cells are horizontal cells. In another embodiment, the ocular cells are ganglion cells. In particular embodiments, the cells are RPE cells.

As used herein, the term “biologically active fragment” in the context of a nucleotide sequence means a nucleotide sequence fragment that retains a biologically active function of the complete nucleotide sequence. For example, where the nucleotide sequence encodes a protein, a biologically active fragment of the nucleotide sequence encodes at least a biologically active fragment of the protein (e.g., a fragment of the protein that retains the function of the protein). Where the nucleotide sequence is the sequence of a virus vector, a biologically active fragment is a fragment that retains the ability of the vector to replicate and be packaged into a virus particle. Where the vector can be used for expression of a protein, a biologically active fragment of the vector retains the ability to express the protein or a biologically active fragment of the protein.

Each embodiment described herein may be used individually or in combination with any other embodiment described herein.

Construction of rAAV Vectors

The disclosure provides AAV vectors comprising a gene encoding CFH polypeptide or a biologically active fragment and/or variant thereof for use in any of the methods disclosed herein. The disclosure provides recombinant AAV (rAAV) vectors comprising a gene encoding CFH polypeptide or a biologically active fragment and/or variant thereof, e.g., under the control of a suitable promoter to direct the expression of the complement system gene, splice variant, or fragment thereof in the eye. The disclosure further provides a therapeutic composition comprising a gene encoding CFH polypeptide or a biologically active fragment and/or variant thereof, e.g. under the control of a suitable promoter. A variety of rAAV vectors may be used to deliver the desired complement system gene to the eye and to direct its expression. More than 30 naturally occurring serotypes of AAV from humans and non-human primates are known. Many natural variants of the AAV capsid exist, and an rAAV vector of the disclosure may be designed based on an AAV with properties specifically suited for ocular cells. In certain embodiments, the CFH polypeptide is a splice variant (e.g. FHL1, which is a truncated splice variant of CFH) or a biologically active fragment and/or variant thereof.

In general, an rAAV vector is comprised of, in order, a 5′ adeno-associated virus inverted terminal repeat, a transgene or gene of interest encoding a CFH polypeptide or a biologically active fragment and/or variant thereof, e.g., operably linked to a sequence which regulates its expression in a target cell, and a 3′ adeno-associated virus inverted terminal repeat. In addition, the rAAV vector may comprise a polyadenylation sequence. Generally, rAAV vectors should have one copy of the AAV ITR at each end of the transgene or gene of interest, in order to allow replication, packaging, and efficient integration into cell chromosomes. Within preferred embodiments of the disclosure, the transgene sequence encoding a CFH polypeptide or a biologically active fragment and/or variant thereof, e.g., will be of about 2 to 5 kb in length (or alternatively, the transgene may additionally contain a “stuffer” or “filler” sequence to bring the total size of the nucleic acid sequence between the two ITRs to between 2 and 5 kb). Alternatively, the transgene encoding a CFH polypeptide or a biologically active fragment and/or variant thereof, e.g., may be composed of the same heterologous sequence several times (e.g., two nucleic acid molecules of a complement system gene separated by a ribosomal readthrough stop codon, or alternatively, by an Internal Ribosome Entry Site or “IRES”), or several different heterologous sequences (e.g., different CFH polypeptides such as FHL1, separated by a ribosomal readthrough stop codon or an IRES).

Recombinant AAV vectors of the present disclosure may be generated from a variety of adeno-associated viruses. For example, ITRs from any AAV serotype are expected to have similar structures and functions with regard to replication, integration, excision and transcriptional mechanisms. Examples of AAV serotypes include AAV1, AAV2, AAV3, AAV4, AAV5, AAV6, AAV7, AAV.7m8, AAV8, AAV9, AAV10, AAV11 and AAV12. In some embodiments, the rAAV vector is generated from serotype AAV1, AAV2, AAV4, AAV5, or AAV8. These serotypes are known to target photoreceptor cells or the retinal pigment epithelium. In particular embodiments, the rAAV vector is generated from serotype AAV2. In particular embodiments, the rAAV vector is generated from serotype AAV.7m8. In certain embodiments, the AAV serotypes include AAVrh8, AAVrh8R or AAVrh10. It will also be understood that the rAAV vectors may be chimeras of two or more serotypes selected from serotypes AAV1 through AAV12. The tropism of the vector may be altered by packaging the recombinant genome of one serotype into capsids derived from another AAV serotype. In some embodiments, the ITRs of the rAAV virus may be based on the ITRs of any one of AAV1-12 and may be combined with an AAV capsid selected from any one of AAV1-12, AAV-DJ, AAV-DJ8, AAV-DJ9 or other modified serotypes. In certain embodiments, any AAV capsid serotype may be used with the vectors of the disclosure. Examples of AAV serotypes include AAV1, AAV2, AAV3, AAV4, AAV5, AAV6, AAV7, AAV8, AAV9, AAV10, AAV11, AAV12, AAV-DJ, AAV-DJ8, AAV-DJ9, AAVrh8, AAVrh8R or AAVrh10. In certain embodiments, the AAV capsid serotype is AAV2. In certain embodiments, the AAV capsid serotype is AAV.7m8 (see, e.g., International Patent Application Publication No. WO 2012/145601).

Desirable AAV fragments for assembly into vectors may include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments may be used, alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the disclosure. In some embodiments, the AAV is AAV2/5. In another embodiment, the AAV is AAV2/8. When pseudotyping an AAV vector, the sequences encoding each of the essential rep proteins may be supplied by different AAV sources (e.g., AAV1, AAV2, AAV3, AAV4, AAV5, AAV6, AAV7, AAV8). For example, the rep78/68 sequences may be from AAV2, whereas the rep52/40 sequences may be from AAV8.

In one embodiment, the vectors of the disclosure contain, at a minimum, sequences encoding a selected AAV serotype capsid, e.g., an AAV2 capsid or a fragment thereof. In another embodiment, the vectors of the disclosure contain, at a minimum, sequences encoding a selected AAV serotype rep protein, e.g., AAV2 rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV2 origin. In certain embodiments, the vectors may comprise rep sequences from an AAV serotype which differs from that which is providing the cap sequences. In some embodiments, the rep and cap sequences are expressed from separate sources (e.g., separate vectors, or a host cell and a vector). In some embodiments, these rep sequences are fused in frame to cap sequences of a different AAV serotype to form a chimeric AAV vector, such as AAV2/8 described in U.S. Pat. No. 7,282,199, which is incorporated by reference herein. Examples of AAV serotypes include AAV1, AAV2, AAV3, AAV4, AAV5, AAV6, AAV7, AAV8, AAV9, AAV10, AAV11, AAV12, AAV-DJ, AAV-DJ8, AAV-DJ9, AAVrh8, AAVrh8R or AAVrh10. In some embodiments, the cap is derived from AAV2. In some embodiments, the cap is derived from AAV.7m8.

In some embodiments, any of the vectors disclosed herein includes a spacer, i.e., a DNA sequence interposed between the promoter and the rep gene ATG start site. In some embodiments, the spacer may be a random sequence of nucleotides, or alternatively, it may encode a gene product, such as a marker gene. In some embodiments, the spacer may contain genes which typically incorporate start/stop and polyA sites. In some embodiments, the spacer may be a non-coding DNA sequence from a prokaryote or eukaryote, a repetitive non-coding sequence, a coding sequence without transcriptional controls or a coding sequence with transcriptional controls. In some embodiments, the spacer is a phage ladder sequence or a yeast ladder sequence. In some embodiments, the spacer is of a size sufficient to reduce expression of the rep78 and rep68 gene products, leaving the rep52, rep40 and cap gene products expressed at normal levels. In some embodiments, the length of the spacer may therefore range from about 10 bp to about 10.0 kbp, preferably in the range of about 100 bp to about 8.0 kbp. In some embodiments, the spacer is less than 2 kbp in length.

In certain embodiments, the AAV capsid is modified to improve therapy. The capsid may be modified using conventional molecular biology techniques. In certain embodiments, the capsid is modified for minimized immunogenicity, better stability and particle lifetime, efficient degradation, and/or accurate delivery of the transgene encoding the CFH polypeptide or a biologically active fragment and/or variant thereof to the nucleus. In some embodiments, the modification or mutation is an amino acid deletion, insertion, substitution, or any combination thereof in a capsid protein. A modified polypeptide may comprise 1, 2, 3, 4, 5, up to 10, or more amino acid substitutions and/or deletions and/or insertions. A “deletion” may comprise the deletion of individual amino acids, deletion of small groups of amino acids such as 2, 3, 4 or 5 amino acids, or deletion of larger amino acid regions, such as the deletion of specific amino acid domains or other features. An “insertion” may comprise the insertion of individual amino acids, insertion of small groups of amino acids such as 2, 3, 4 or 5 amino acids, or insertion of larger amino acid regions, such as the insertion of specific amino acid domains or other features. A “substitution” comprises replacing a wild type amino acid with another (e.g., a non-wild type amino acid). In some embodiments, the another (e.g., non-wild type) or inserted amino acid is Ala (A), His (H), Lys (K), Phe (F), Met (M), Thr (T), Gln (Q), Asp (D), or Glu (E). In some embodiments, the another (e.g., non-wild type) or inserted amino acid is A. In some embodiments, the another (e.g., non-wild type) amino acid is Arg (R), Asn (N), Cys (C), Gly (G), Ile (I), Leu (L), Pro (P), Ser (S), Trp (W), Tyr (Y), or Val (V). Conventional or naturally occurring amino acids are divided into the following basic groups based on common side-chain properties: (1) non-polar: Norleucine, Met, Ala, Val, Leu, He; (2) polar without charge: Cys, Ser, Thr, Asn, Gin; (3) acidic (negatively charged): Asp, Glu; (4) basic (positively charged): Lys, Arg; and (5) residues that influence chain orientation: Gly, Pro; and (6) aromatic: Trp, Tyr, Phe, His. Conventional amino acids include L or D stereochemistry. In some embodiments, the another (e.g., non-wild type) amino acid is a member of a different group (e.g., an aromatic amino acid is substituted for a non-polar amino acid). Substantial modifications in the biological properties of the polypeptide are accomplished by selecting substitutions that differ significantly in their effect on maintaining (a) the structure of the polypeptide backbone in the area of the substitution, for example, as a R-sheet or helical conformation, (b) the charge or hydrophobicity of the molecule at the target site, or (c) the bulk of the side chain. Naturally occurring residues are divided into groups based on common side-chain properties: (1) Non-polar: Norleucine, Met, Ala, Val, Leu, Ile; (2) Polar without charge: Cys, Ser, Thr, Asn, Gln; (3) Acidic (negatively charged): Asp, Glu; (4) Basic (positively charged): Lys, Arg; (5) Residues that influence chain orientation: Gly, Pro; and (6) Aromatic: Trp, Tyr, Phe, His. In some embodiments, the another (e.g., non-wild type) amino acid is a member of a different group (e.g., a hydrophobic amino acid for a hydrophilic amino acid, a charged amino acid for a neutral amino acid, an acidic amino acid for a basic amino acid, etc.). In some embodiments, the another (e.g., non-wild type) amino acid is a member of the same group (e.g., another basic amino acid, another acidic amino acid, another neutral amino acid, another charged amino acid, another hydrophilic amino acid, another hydrophobic amino acid, another polar amino acid, another aromatic amino acid or another aliphatic amino acid). In some embodiments, the another (e.g., non-wild type) amino acid is an unconventional amino acid. Unconventional amino acids are non-naturally occurring amino acids. Examples of an unconventional amino acid include, but are not limited to, aminoadipic acid, beta-alanine, beta-aminopropionic acid, aminobutyric acid, piperidinic acid, aminocaprioic acid, aminoheptanoic acid, aminoisobutyric acid, aminopimelic acid, citrulline, diaminobutyric acid, desmosine, diaminopimelic acid, diaminopropionic acid, N-ethylglycine, N-ethylaspargine, hyroxylysine, allo-hydroxylysine, hydroxyproline, isodesmosine, allo-isoleucine, N-methylglycine, sarcosine, N-methylisoleucine, N-methylvaline, norvaline, norleucine, orithine, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and amino acids (e.g., 4-hydroxyproline). In some embodiments, one or more amino acid substitutions are introduced into one or more of VP1, VP2 and VP3. In one aspect, a modified capsid protein comprises 1, 2, 3, 4, 5, 6, 7, 8, 9, 10, 11, 12, 13, 14, or 15 conservative or non-conservative substitutions relative to the wild-type polypeptide. In another aspect, the modified capsid polypeptide of the disclosure comprises modified sequences, wherein such modifications can include both conservative and non-conservative substitutions, deletions, and/or additions, and typically include peptides that share at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the corresponding wild-type capsid protein.

In some embodiments, the recombinant AAV vector, rep sequences, cap sequences, and helper functions required for producing the rAAV of the disclosure may be delivered to the packaging host cell using any appropriate genetic element (vector). In some embodiments, a single nucleic acid encoding all three capsid proteins (e.g., VP1, VP2 and VP3) is delivered into the packaging host cell in a single vector. In some embodiments, nucleic acids encoding the capsid proteins are delivered into the packaging host cell by two vectors; a first vector comprising a first nucleic acid encoding two capsid proteins (e.g., VP1 and VP2) and a second vector comprising a second nucleic acid encoding a single capsid protein (e.g., VP3). In some embodiments, three vectors, each comprising a nucleic acid encoding a different capsid protein, are delivered to the packaging host cell. The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). In some embodiments, vectors suitable for use with the present disclosure may be pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Cells may also be transfected with a vector (e.g., helper vector) which provides helper functions to the AAV. The vector providing helper functions may provide adenovirus functions, including, e.g., E1a, E1b, E2a, E40RF6. The sequences of an adenovirus gene providing these functions may be obtained from any known adenovirus serotype, such as serotypes 2, 3, 4, 7, 12 and 40, and further including any of the presently identified human types known in the art. Thus, in some embodiments, the methods involve transfecting the cell with a vector expressing one or more genes necessary for AAV replication, AAV gene transcription, and/or AAV packaging.

An rAAV vector of the disclosure is generated by introducing a nucleic acid sequence encoding an AAV capsid protein, or fragment thereof; a functional rep gene or a fragment thereof; a minigene composed of, at a minimum, AAV inverted terminal repeats (ITRs) and a transgene encoding a CFH polypeptide or a biologically active fragment and/or variant thereof; and sufficient helper functions to permit packaging of the minigene into the AAV capsid, into a host cell. The components required for packaging an AAV minigene into an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

In some embodiments, such a stable host cell will contain the required component(s) under the control of an inducible promoter. Alternatively, the required component(s) may be under the control of a constitutive promoter. Examples of suitable inducible and constitutive promoters are provided herein, in the discussion below of regulator elements suitable for use with the transgene, i.e., a nucleic acid encoding a CFH polypeptide or biologically active fragment and/or variant thereof. In still another alternative, a selected stable host cell may contain selected components under the control of a constitutive promoter and other selected components under the control of one or more inducible promoters.

The minigene, rep sequences, cap sequences, and helper functions required for producing the rAAV of the disclosure may be delivered to the packaging host cell in the form of any genetic element which transfers the sequences. The selected genetic element may be delivered by any suitable method known in the art. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, 1993 J. Virol, 70:520-532 and U.S. Pat. No. 5,478,745, among others. These publications are incorporated by reference herein.

Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV.7m8, AAV8, AAV9, AAV10, AAV10, AAV11, AAV12, AAV-DJ, AAV-DJ8, AAV-DJ9, AAVrh8, AAVrh8R or AAVrh10 or other known and unknown AAV serotypes. These ITRs or other AAV components may be readily isolated using techniques available to those of skill in the art from an AAV serotype. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

The minigene is composed of, at a minimum, a transgene encoding a CFH polypeptide or a biologically active fragment and/or variant thereof, and its regulatory sequences, and 5′ and 3′ AAV inverted terminal repeats (ITRs). In one desirable embodiment, the ITRs of AAV serotype 2 are used. However, ITRs from other suitable serotypes may be selected. The minigene is packaged into a capsid protein and delivered to a selected host cell.

In some embodiments, regulatory sequences are operably linked to the transgene encoding a CFH polypeptide or a biologically active fragment and/or variant thereof. The regulatory sequences may include conventional control elements which are operably linked to the complement system gene, splice variant, or a fragment thereof in a manner which permits its transcription, translation and/or expression in a cell transfected with the vector or infected with the virus produced by the disclosure. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Numerous expression control sequences, including promoters, are known in the art and may be utilized.

The regulatory sequences useful in the constructs of the present disclosure may also contain an intron, desirably located between the promoter/enhancer sequence and the gene. In some embodiments, the intron sequence is derived from SV-40, and is a 100 bp mini-intron splice donor/splice acceptor referred to as SD-SA. Another suitable sequence includes the woodchuck hepatitis virus post-transcriptional element. (See, e.g., L. Wang and I. Verma, 1999 Proc. Natl. Acad. Sci., USA, 96:3906-3910). PolyA signals may be derived from many suitable species, including, without limitation SV-40, human and bovine.

Another regulatory component of the rAAV that may be useful in the method of the disclosure is an internal ribosome entry site (IRES). An IRES sequence, or other suitable systems, may be used to produce more than one polypeptide from a single gene transcript (for example, to produce more than one complement system polypeptides). An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ to the transgene in the rAAV vector.

In some embodiments, expression of the transgene encoding a CFH polypeptide or a biologically active fragment and/or variant thereof is driven by a separate promoter (e.g., a viral promoter). In certain embodiments, any promoters suitable for use in AAV vectors may be used with the vectors of the disclosure. The selection of the transgene promoter to be employed in the rAAV may be made from among a wide number of constitutive or inducible promoters that can express the selected transgene in the desired ocular cell. Examples of suitable promoters are described herein.

Other regulatory sequences useful in the disclosure include enhancer sequences. Enhancer sequences useful in the disclosure include the 1RBP enhancer (Nicoud 2007, cited above), immediate early cytomegalovirus enhancer, one derived from an immunoglobulin gene or SV40 enhancer, the cis-acting element identified in the mouse proximal promoter, etc.

Selection of these and other common vector and regulatory elements are well-known and many such sequences are available. See, e.g., Sambrook et al, and references cited therein at, for example, pages 3.18-3.26 and 16, 17-16.27 and Ausubel et al., Current Protocols in Molecular Biology, John Wiley & Sons, New York, 1989).

The rAAV vector may also contain additional sequences, for example from an adenovirus, which assist in effecting a desired function for the vector. Such sequences include, for example, those which assist in packaging the rAAV vector in adenovirus-associated virus particles.

The rAAV vector may also contain a reporter sequence for co-expression, such as but not limited to lacZ, GFP, CFP, YFP, RFP, mCherry, tdTomato, etc. In some embodiments, the rAAV vector may comprise a selectable marker. In some embodiments, the selectable marker is an antibiotic-resistance gene. In some embodiments, the antibiotic-resistance gene is a kanamycin-resistance gene. In some embodiments, the antibiotic-resistance gene is an ampicillin-resistance gene. In some embodiments, the ampicillin-resistance gene is beta-lactamase.

In some embodiments, the rAAV particle is an ssAAV. In some embodiments, the rAAV particle is a self-complementary AAV (sc-AAV) (See, US 2012/0141422 which is incorporated herein by reference). Self-complementary vectors package an inverted repeat genome that can fold into dsDNA without the requirement for DNA synthesis or base-pairing between multiple vector genomes. Because scAAV have no need to convert the single-stranded DNA (ssDNA) genome into double-stranded DNA (dsDNA) prior to expression, they are more efficient vectors. However, the trade-off for this efficiency is the loss of half the coding capacity of the vector, scAAV are useful for small protein-coding genes (up to 55 kd) and any currently available RNA-based therapy.

rAAV vectors useful in the methods of the disclosure are further described in PCT publication No. WO2015168666 and PCT publication no. WO2014011210, the contents of which are incorporated by reference herein.

In some embodiments, any of the vectors disclosed herein is capable of inducing at least 20%, at least 50%, at least 100%, at least 150%, at least 200%, at least 250%, at least 300%, at least 400%, at least 500%, at least 700%, at least 900%, at least 1000%, at least 1100%, at least 1500%, or at least 2000% expression of CFH or FHL in a target cell (e.g., an RPE or liver cell) as compared to the endogenous expression of CFH or FHL in the target cell. In some embodiments, expression of any of the vectors disclosed herein in a target cell (e.g., an RPE or liver cell) results in at least 20%, at least 50%, at least 100%, at least 150%, at least 200%, at least 250%, at least 300%, at least 400%, at least 500%, at least 700%, at least 900%, at least 1000%, at least 1100%, at least 1500%, or at least 2000% levels of CFH or FHL activity in the target cell as compared to endogenous levels of CFH or FHL activity in the target cell.

It is understood that CFH is a secreted protein, and its expression can also be determined by measuring the level of CFH in an extracellular compartment. For example, in some embodiments, the rAAV vector is administered intraocularly (e.g., intravitreally), and the level of the CFH expressed from the rAAV vector in the aqueous humor or vitreous humor is at least 50 ng/mL, at least 55 ng/mL, at least 60 ng/mL, at least 65 ng/mL, at least 70 ng/mL, at least 75 ng/mL, at least 80 ng/mL, at least 85 ng/mL, at least 90 ng/mL, or at least 100 ng/mL. In some embodiments, the rAAV vector is administered intraocularly (e.g., intravitreally), and the level of the CFH in the aqueous humor or vitreous humor is at least 50 ng/mL, at least 55 ng/mL, at least 60 ng/mL, at least 65 ng/mL, at least 70 ng/mL, at least 75 ng/mL, at least 80 ng/mL, at least 85 ng/mL, at least 90 ng/mL, or at least 100 ng/mL. In some embodiments, the rAAV vector is administered systemically (e.g., intravenously), and the level of the CFH expressed from the rAAV vector in the plasma of the subject is at least 100 μg/mL, at least 150 μg/mL, at least 200 μg/mL, at least 250 μg/mL, at least 300 μg/mL, at least 350 μg/mL, at least 400 μg/mL, at least 450 μg/mL, or at least 500 μg/mL. In some embodiments, the rAAV vector is administered systemically (e.g., intravenously), and the level of the CFH in the plasma of the subject is at least 100 μg/mL, at least 150 μg/mL, at least 200 μg/mL, at least 250 μg/mL, at least 300 μg/mL, at least 350 μg/mL, at least 400 μg/mL, at least 450 μg/mL, or at least 500 μg/mL.

Examples of specific CFH-AAV constructs are described in WO 2019/079718, which is incorporated by reference herein in its entirety.

### Complement System Genes

The disclosure provides a Complement Factor H (CFH) polypeptide or a biologically active fragment and/or variant thereof for use in any of the methods disclosed herein.

Bruch's membrane is a sheet of extracellular matrix that separates the retina from the underlying choroid, a highly vascularized layer that supplies oxygen and nutrition to the outer retina. Proteins up to 100 kDa can pass across Bruch's membrane, and proteins larger than 100 kDa can pass across to a variable extent. Additionally, the permeability of Bruch's membrane decreases with aging. In some embodiments, any of the vectors disclosed herein encoding any of the CFH polypeptides or biologically active fragment and/or variant thereof disclosed herein is capable of diffusing across the Bruch's membrane. This may be accomplished by varying one or more of the following parameters: hydrodynamic size, dynamic radius, shape, post-translational modifications (e.g. glycosylation), net charge or propensity for the polypeptide to interact with Bruch's membrane components.

To prevent host cell and tissue destruction, the alternative pathway must be tightly controlled using a group of alternative pathway regulators. One endogenous mechanism often employed to regulate excessive alternative pathway activity is to prevent convertase formation completely by degrading the core component C3b to a proteolytic by-product that is incapable of forming the convertase. The C3b-degrading activity is primarily catalyzed by the protease complement factor I (CFI). However, CFI needs to form a binary complex with a second protein such as CFH in order to display its catalytic function. In some embodiments, any of the vectors disclosed herein encodes a CFH polypeptide or biologically active fragment and/or variant thereof that is functional. In some embodiments, the CFH polypeptide or biologically active fragment and/or variant thereof is capable of binding C3b. In some embodiments, the binding of CFH to C3b prevents the formation of a membrane attack complex. In some embodiments, the CFH polypeptide or biologically active fragment and/or variant thereof is capable of facilitating the breakdown of C3b. In some embodiments, the CFH polypeptide or biologically active fragment and/or variant thereof is capable of destabilizing C3bBb. In some embodiments, the CFH polypeptide or biologically active fragment and/or variant thereof competes with factor B for binding to C3b. In some embodiments, the CFH polypeptide or biologically active fragment and/or variant thereof prevents the formation of a C3 convertase (e.g. C3bBb). In some embodiments, the CFH polypeptide or biologically active fragment and/or variant thereof accelerates the decay of convertase complexes (e.g. C3bBb). In some embodiments, the CFH polypeptide or biologically active fragment and/or variant thereof accelerates the decay of the alternative pathway C5 convertase (C3b2Bb). In some embodiments, the CFH polypeptide or biologically active fragment and/or variant thereof is capable of suppressing C3b amplification. In some embodiments, the CFH polypeptide or biologically active fragment and/or variant thereof is capable of binding to a cell surface (e.g., an erythrocyte and/or endothelial cell). In some embodiments, the CFH polypeptide or biologically active fragment and/or variant thereof is capable of binding to heparin.

Human mature wildtype CFH is a 1213 amino acid soluble protein which comprises 20 complement-control protein modules (CCPs 1-20), which are approximately 60 amino acid residues in length. Alignment of the 20 CCPs demonstrates four invariant cysteine residues arranged in two conserved disulfide bonds, and a near-invariant tryptophan residue. Short three to eight amino acid residue “linkers” are found between the last residue of one CCP and the first residue of the next CCP. Each of the CCPs fold into a distinct three-dimensional 3-sheet rich structure (Schmidt C. Q. Clin Exp Immunol. 2008; 151(1):14-24).

In some embodiments, any of the vectors disclosed herein encodes a CFH polypeptide comprising at least one CCP module or a biologically active fragment and/or variant thereof. In some embodiments, the CFH polypeptide comprises at least two CCP modules or a biologically active fragment and/or variant thereof. In some embodiments, the CFH polypeptide comprises at least three CCP modules or a biologically active fragment and/or variant thereof. In some embodiments, the CFH polypeptide comprises at least four CCP modules or a biologically active fragment and/or variant thereof. In some embodiments, the CFH polypeptide comprises at least five CCP modules or a biologically active fragment and/or variant thereof. In some embodiments, the CFH polypeptide comprises at least six CCP modules or a biologically active fragment and/or variant thereof. In some embodiments, the CFH polypeptide comprises at least seven CCP modules or a biologically active fragment and/or variant thereof. In some embodiments, the CFH polypeptide comprises at least eight CCP modules or a biologically active fragment and/or variant thereof. In some embodiments, the CFH polypeptide comprises at least nine CCP modules or a biologically active fragment and/or variant thereof. In some embodiments, the CFH polypeptide comprises at least ten CCP modules or a biologically active fragment and/or variant thereof. In some embodiments, the CFH polypeptide comprises at least eleven CCP modules or a biologically active fragment and/or variant thereof. In some embodiments, the CFH polypeptide comprises at least twelve CCP modules or a biologically active fragment and/or variant thereof. In some embodiments, the CFH polypeptide comprises at least thirteen CCP modules or a biologically active fragment and/or variant thereof. In some embodiments, the CFH polypeptide comprises at least fourteen CCP modules or a biologically active fragment and/or variant thereof. In some embodiments, the CFH polypeptide comprises at least fifteen CCP modules or a biologically active fragment and/or variant thereof. In some embodiments, the CFH polypeptide comprises at least sixteen CCP modules or a biologically active fragment and/or variant thereof. In some embodiments, the CFH polypeptide comprises at least seventeen CCP modules or a biologically active fragment and/or variant thereof. In some embodiments, the CFH polypeptide comprises at least eighteen CCP modules or a biologically active fragment and/or variant thereof. In some embodiments, the CFH polypeptide comprises at least nineteen CCP modules or a biologically active fragment and/or variant thereof. In some embodiments, the CFH polypeptide comprises twenty CCP modules or a biologically active fragment and/or variant thereof.

In some embodiments, the CFH polypeptide encoded by any of the vectors disclosed herein comprises any one of or any combination of CCP modules 1, 2, 3, 4, 5, 6, 7, 8, 9, 10, 11, 12, 13, 14, 15, 16, 17, 18, 19, and/or 20 or a biologically active fragment and/or variant thereof. In some embodiments, the CFH polypeptide comprises at least one of CCP modules 1, 2, 3, 4, 5, 6, 7, 8, 9, 10, 11, 12, 13, 14, 15, 16, 17, 18, 19, and/or 20 or a biologically active fragment and/or variant thereof. In some embodiments, the CFH polypeptide comprises CCP modules 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, or 1-20 or a biologically active fragment and/or variant thereof. In some embodiments, the CFH polypeptide comprises CCP modules 2-20, 3-20, 4-20, 5-20, 6-20, 7-20, 8-20, 9-20, 10-20, 11-20, 12-20, 13-20, 14-20, 15-20, 16-20, 17-20, 18-20, or 19-20 or a biologically active fragment and/or variant thereof. In some embodiments, the CFH polypeptide comprises CCP modules 1-2 and 19-20, or a biologically active fragment and/or variant thereof. In some embodiments, the CFH polypeptide comprises CCP modules 1-4 and 19-20, or a biologically active fragment and/or variant thereof. In some embodiments, the CFH polypeptide comprises CCP modules 1-2 and 18-20, or a biologically active fragment and/or variant thereof. In some embodiments, the CFH polypeptide comprises CCP modules 1-4 and 18-20, or a biologically active fragment and/or variant thereof.

In some embodiments, the CFH polypeptide encoded by any of the vectors disclosed herein comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 33, or a biologically active fragment thereof. In some embodiments, the CFH polypeptide comprises an amino acid sequence that is at least 90% identical to the amino acid sequence of SEQ ID NO: 33, or a biologically active fragment thereof. In some embodiments, the CFH polypeptide comprises an amino acid sequence that is at least 95% identical to the amino acid sequence of SEQ ID NO: 33, or a biologically active fragment thereof.

In some embodiments, the CFH polypeptide encoded by any of the vectors disclosed herein comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 34, or a biologically active fragment thereof. In some embodiments, the CFH polypeptide comprises an amino acid sequence that is at least 90% identical to the amino acid sequence of SEQ ID NO: 34, or a biologically active fragment thereof. In some embodiments, the CFH polypeptide comprises an amino acid sequence that is at least 95% identical to the amino acid sequence of SEQ ID NO: 34, or a biologically active fragment thereof.

In some embodiments, the CFH polypeptide encoded by any of the vectors disclosed herein comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 37, or a biologically active fragment thereof. In some embodiments, the CFH polypeptide comprises an amino acid sequence that is at least 90% identical to the amino acid sequence of SEQ ID NO: 37, or a biologically active fragment thereof. In some embodiments, the CFH polypeptide comprises an amino acid sequence that is at least 95% identical to the amino acid sequence of SEQ ID NO: 37, or a biologically active fragment thereof.

In some embodiments, the CFH polypeptide encoded by any of the vectors disclosed herein comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 38, or a biologically active fragment thereof. In some embodiments, the CFH polypeptide comprises an amino acid sequence that is at least 90% identical to the amino acid sequence of SEQ ID NO: 38, or a biologically active fragment thereof. In some embodiments, the CFH polypeptide comprises an amino acid sequence that is at least 95% identical to the amino acid sequence of SEQ ID NO: 38, or a biologically active fragment thereof.

In some embodiments, the CFH polypeptide encoded by any of the vectors disclosed herein comprises at least CCP modules 1-4 or a biologically active fragment and/or variant thereof. In some embodiments, the CFH polypeptide comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 39, or a biologically active fragment thereof. In some embodiments, the CFH polypeptide comprises an amino acid sequence that is at least 90% identical to the amino acid sequence of SEQ ID NO: 39, or a biologically active fragment thereof. In some embodiments, the CFH polypeptide comprises an amino acid sequence that is at least 95% identical to the amino acid sequence of SEQ ID NO: 39, or a biologically active fragment thereof.

In some embodiments, the CFH polypeptide encoded by any of the vectors disclosed herein comprises at least CCP modules 1-5 or a biologically active fragment and/or variant thereof. In some embodiments, the CFH polypeptide comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 40, or a biologically active fragment thereof. In some embodiments, the CFH polypeptide comprises an amino acid sequence that is at least 90% identical to the amino acid sequence of SEQ ID NO: 40, or a biologically active fragment thereof. In some embodiments, the CFH polypeptide comprises an amino acid sequence that is at least 95% identical to the amino acid sequence of SEQ ID NO: 40, or a biologically active fragment thereof.

In some embodiments, the CFH polypeptide encoded by any of the vectors disclosed herein comprises at least CCP modules 1-7 or a biologically active fragment and/or variant thereof. In some embodiments, the CFH polypeptide comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 41, or a biologically active fragment thereof. In some embodiments, the CFH polypeptide comprises an amino acid sequence that is at least 90% identical to the amino acid sequence of SEQ ID NO: 41, or a biologically active fragment thereof. In some embodiments, the CFH polypeptide comprises an amino acid sequence that is at least 95% identical to the amino acid sequence of SEQ ID NO: 41, or a biologically active fragment thereof.

In some embodiments, the CFH polypeptide encoded by any of the vectors disclosed herein comprises at least CCP modules 19-20 or a biologically active fragment and/or variant thereof. In some embodiments, the CFH polypeptide comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 42, or a biologically active fragment thereof. In some embodiments, the CFH polypeptide comprises an amino acid sequence that is at least 90% identical to the amino acid sequence of SEQ ID NO: 42, or a biologically active fragment thereof. In some embodiments, the CFH polypeptide comprises an amino acid sequence that is at least 95% identical to the amino acid sequence of SEQ ID NO: 42, or a biologically active fragment thereof.

In some embodiments, the CFH polypeptide encoded by any of the vectors disclosed herein comprises at least CCP modules 18-20 or a biologically active fragment and/or variant thereof. In some embodiments, the CFH polypeptide comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 43, or a biologically active fragment thereof. In some embodiments, the CFH polypeptide comprises an amino acid sequence that is at least 90% identical to the amino acid sequence of SEQ ID NO: 43, or a biologically active fragment thereof. In some embodiments, the CFH polypeptide comprises an amino acid sequence that is at least 95% identical to the amino acid sequence of SEQ ID NO: 43, or a biologically active fragment thereof.

In some embodiments, the vector comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 1, or a biologically active fragment thereof.

In some embodiments, the vector comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 2, or a biologically active fragment thereof.

In some embodiments, the vector comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 3, or a biologically active fragment thereof.

In some embodiments, the vector comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 5, or a biologically active fragment thereof.

In some embodiments, the CFH biologically active fragment and/or variant encoded by any of the vectors disclosed herein is capable of inducing any one or more of the effects listed above with regard to a wildtype CFH protein. For example, in some embodiments, the biologically active fragment and/or variant of CFH is capable of acting as a cofactor with CFI to facilitate C3b cleavage. In some embodiments, the CFH protein or biologically active fragment and/or variant thereof is capable of diffusing across the Bruch's membrane. In some embodiments, the CFH protein or biologically active fragment and/or variant thereof is capable of binding C3b. In some embodiments, the CFH protein or biologically active fragment and/or variant thereof is capable of facilitating the breakdown of C3b. In some embodiments, the CFH protein or biologically active fragment and/or variant thereof is capable of binding to a cell surface (e.g., an erythrocyte and/or endothelial cell). In some embodiments, the CFH protein or biologically active fragment and/or variant thereof is capable of binding to heparin. In some embodiments, the CFH protein or biologically active fragment and/or variant thereof is capable of reducing C5b9 levels generated as a result of complement activation (e.g., as measured in a Wieslab AP assay (see, e.g., Example 1). In some embodiments, the CFH protein or biologically active and/or variant fragment thereof is capable of inhibiting hemolysis. In some embodiments, the biologically active fragment and/or variant is at least 200, at least 250, at least 300, at least 350, at least 400, at least 450, at least 500, at least 600, at least 700, at least 800, at least 900, at least 1000, at least 1100, at least 1200, or at least 1213 amino acids in length. In some embodiments, the biologically active fragment and/or variant is between 100-1213, between 200-1213, between 300-1213, between 400-1213, between 500-1213, between 600-1213, between 700-1213, between 800-1213, between 900-1213, between 1000-1213, between 1100-1213, between 500-1213, between 100-200, between 100-300, between 100-400, between 100-500, between 100-600, between 100-700, between 100-800, between 100-900, between 200-300, between 200-400, between 200-500, between 200-600, between 200-700, between 200-800, between 200-900, between 300-400, between 300-500, between 300-600, between 300-700, between 300-800, between 300-900, between 400-500, between 400-600, between 400-700, between 400-800, between 400-900, between 500-600, between 500-700, between 500-800, between 500-900, between 600-700, between 600-800, between 600-900, between 700-800, between 700-900, or between 800-900 amino acids in length. In some embodiments, the biologically active fragment comprises at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, or at least 1200 consecutive amino acids from a sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 33, 34, 37 or 38 (e.g., SEQ ID NO: 38). In some embodiments, the biologically active fragment and/or variant comprises at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, or at least 1200 consecutive amino acids of SEQ ID NO: 33, 34, 37, or 38 (e.g., SEQ ID NO: 38).

In some embodiments, any of the CFH polypeptides or biologically active fragments and/or variants disclosed herein encoded by any of the vectors disclosed herein is a modified CFH polypeptide or biologically active fragment thereof as compared to a reference CFH sequence (e.g., a protein comprising the amino acid sequence of any one of SEQ ID NOs: 33-34 or 37-38). In some embodiments, a modified CFH polypeptide may comprise 1, 2, 3, 4, 5, up to 10, or more amino acid substitutions and/or deletions and/or insertions. A “deletion” may comprise the deletion of individual amino acids, deletion of small groups of amino acids such as 2, 3, 4 or 5 amino acids, or deletion of larger amino acid regions, such as the deletion of specific amino acid domains (e.g., one or more CCP domains) or other features. An “insertion” may comprise the insertion of individual amino acids, insertion of small groups of amino acids such as 2, 3, 4 or 5 amino acids, or insertion of larger amino acid regions, such as the insertion of specific amino acid domains or other features (e.g., insertion of a linker). A “substitution” comprises replacing a wild type amino acid with another (e.g., a non-wild type amino acid). In some embodiments, the another (e.g., non-wild type) or inserted amino acid is Ala (A), His (H), Lys (K), Phe (F), Met (M), Thr (T), Gln (Q), Asp (D), or Glu (E). In some embodiments, the another (e.g., non-wild type) or inserted amino acid is A. In some embodiments, the another (e.g., non-wild type) amino acid is Arg (R), Asn (N), Cys (C), Gly (G), Ile (I), Leu (L), Pro (P), Ser (S), Trp (W), Tyr (Y), or Val (V). Conventional or naturally occurring amino acids are divided into the following basic groups based on common side-chain properties: (1) non-polar: Norleucine, Met, Ala, Val, Leu, He; (2) polar without charge: Cys, Ser, Thr, Asn, Gin; (3) acidic (negatively charged): Asp, Glu; (4) basic (positively charged): Lys, Arg; and (5) residues that influence chain orientation: Gly, Pro; and (6) aromatic: Trp, Tyr, Phe, His. Conventional amino acids include L or D stereochemistry. In some embodiments, the another (e.g., non-wild type) amino acid is a member of a different group (e.g., an aromatic amino acid is substituted for a non-polar amino acid). Substantial modifications in the biological properties of the polypeptide are accomplished by selecting substitutions that differ significantly in their effect on maintaining (a) the structure of the polypeptide backbone in the area of the substitution, for example, as a R-sheet or helical conformation, (b) the charge or hydrophobicity of the molecule at the target site, or (c) the bulk of the side chain. Naturally occurring residues are divided into groups based on common side-chain properties: (1) Non-polar: Norleucine, Met, Ala, Val, Leu, Ile; (2) Polar without charge: Cys, Ser, Thr, Asn, Gln; (3) Acidic (negatively charged): Asp, Glu; (4) Basic (positively charged): Lys, Arg; (5) Residues that influence chain orientation: Gly, Pro; and (6) Aromatic: Trp, Tyr, Phe, His. In some embodiments, the another (e.g., non-wild type) amino acid is a member of a different group (e.g., a hydrophobic amino acid for a hydrophilic amino acid, a charged amino acid for a neutral amino acid, an acidic amino acid for a basic amino acid, etc.). In some embodiments, the another (e.g., non-wild type) amino acid is a member of the same group (e.g., another basic amino acid, another acidic amino acid, another neutral amino acid, another charged amino acid, another hydrophilic amino acid, another hydrophobic amino acid, another polar amino acid, another aromatic amino acid or another aliphatic amino acid). In some embodiments, the another (e.g., non-wild type) amino acid is an unconventional amino acid. Unconventional amino acids are non-naturally occurring amino acids. Examples of an unconventional amino acid include, but are not limited to, aminoadipic acid, beta-alanine, beta-aminopropionic acid, aminobutyric acid, piperidinic acid, aminocaprioic acid, aminoheptanoic acid, aminoisobutyric acid, aminopimelic acid, citrulline, diaminobutyric acid, desmosine, diaminopimelic acid, diaminopropionic acid, N-ethylglycine, N-ethylaspargine, hyroxylysine, allo-hydroxylysine, hydroxyproline, isodesmosine, allo-isoleucine, N-methylglycine, sarcosine, N-methylisoleucine, N-methylvaline, norvaline, norleucine, orithine, 4-hydroxyproline, γ-carboxyglutamate, ε-N,N,N-trimethyllysine, ε-N-acetyllysine, 0-phosphoserine, N-acetylserine, N-formylmethionine, 3-methylhistidine, 5-hydroxylysine, σ-N-methylarginine, and other similar amino acids and amino acids (e.g., 4-hydroxyproline). In one aspect, a modified CFH protein or biologically active fragment thereof comprises 1, 2, 3, 4, 5, 6, 7, 8, 9, 10, 11, 12, 13, 14, or 15 conservative or non-conservative substitutions relative to the wild-type CFH polypeptide or biologically active fragment thereof (e.g., a polypeptide having the amino acid sequence of any one of SEQ ID NOs: 33-34 or 37-38).

In some embodiments, the CFH polypeptide or biologically active fragment and/or variant thereof encoded by any of the vectors disclosed herein is a precursor CFH polypeptide or biologically active fragment and/or variant thereof. In some embodiments, the precursor CFH polypeptide or biologically active fragment and/or variant thereof is processed to a mature CFH polypeptide or biologically active fragment and/or variant thereof after administration to a subject. In some embodiments, the CFH polypeptide or biologically active fragment and/or variant thereof is a mature CFH polypeptide or biologically active fragment and/or variant thereof.

In certain embodiments, any of the CFH polypeptides or biologically active fragments and/or variants disclosed herein encoded by any of the vectors disclosed herein may further comprise post-translational modifications in addition to any that are naturally present in the native polypeptides. Such modifications include, but are not limited to, acetylation, carboxylation, glycosylation, phosphorylation, lipidation, pegylation (polyethylene glycol) and acylation. As a result, the modified polypeptides may contain non-amino acid elements, such as polyethylene glycols, lipids, mono- or poly-saccharides, and phosphates. Effects of such non-amino acid elements on the functionality of a polypeptide may be tested as described herein for other polypeptide variants. When a polypeptide is produced in cells by cleaving a nascent form of the polypeptide, post-translational processing may also be important for correct folding and/or function of the protein. Different cells have specific cellular machinery and characteristic mechanisms for such post-translational activities and may be chosen to ensure the correct modification and processing of the polypeptides.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present disclosure. Further, alleles of the genes comprising the polynucleotide sequences provided herein are within the scope of the present disclosure. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The nucleic acids/polynucleotides of this disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence. In other embodiments, nucleic acids of the disclosure also include nucleotide sequences that hybridize under highly stringent conditions to the nucleotide sequences set forth in SEQ ID NOs: 1, 2, 3 and 5, or sequences complementary thereto. One of ordinary skill in the art will readily understand that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. One skilled in the art will appreciate that these variations in one or more nucleotides (up to about 3-5% of the nucleotides) of the nucleic acids encoding a particular protein may exist among members of a given species due to natural allelic variation. Any and all such nucleotide variations and resulting amino acid polymorphisms are within the scope of this disclosure.

The present disclosure further provides oligonucleotides that hybridize to a polynucleotide having the nucleotide sequence set forth in SEQ ID NOs: 1, 2, 3 and 5, or to a polynucleotide molecule having a nucleotide sequence which is the complement of a sequence listed above. Such oligonucleotides are at least about 10 nucleotides in length, and preferably from about 15 to about 30 nucleotides in length, and hybridize to one of the aforementioned polynucleotide molecules under highly stringent conditions, i.e., washing in 6×SSC/0.5% sodium pyrophosphate at about 37° C. for about 14-base oligos, at about 48° C. for about 17-base oligos, at about 55° C. for about 20-base oligos, and at about 60° C. for about 23-base oligos. In a preferred embodiment, the oligonucleotides are complementary to a portion of one of the aforementioned polynucleotide molecules. These oligonucleotides are useful for a variety of purposes including encoding or acting as antisense molecules useful in gene regulation, or as primers in amplification of complement system-encoding polynucleotide molecules.

In another embodiment, the transgenes useful in any of the methods disclosed herein include reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc. These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

The regulatory sequences include conventional control elements which are operably linked to the transgene encoding a CFH polypeptide or biologically active fragment and/or variant thereof in a manner which permits its transcription, translation and/or expression in a cell transfected with the vector or infected with the virus produced as described herein. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters, are known in the art and may be utilized.

The regulatory sequences useful in any of the constructs/vectors disclosed herein may also contain an intron, desirably located between the promoter/enhancer sequence and the gene. One desirable intron sequence is derived from SV-40, and is a 100 bp mini-intron splice donor/splice acceptor referred to as SD-SA. In some embodiments, the intron comprises the nucleotide sequence of SEQ ID NO: 10, or a codon-optimized or fragment thereof. Another suitable sequence includes the woodchuck hepatitis virus post-transcriptional element. (See, e.g., L. Wang and I. Verma, 1999 Proc. Natl. Acad. Sci., USA, 96:3906-3910). PolyA signals may be derived from many suitable species, including, without limitation SV-40, human and bovine.

Another regulatory component of the rAAV useful in the methods described herein is an internal ribosome entry site (IRES). An IRES sequence, or other suitable systems, may be used to produce more than one polypeptide from a single gene transcript. An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ to the transgene in the rAAV vector.

In one embodiment, the AAV comprises a promoter (or a functional fragment of a promoter). The selection of the promoter to be employed in the rAAV may be made from among a wide number of promoters that can express the selected transgene in the desired target cell. In one embodiment, the target cell is an ocular cell. In some embodiments, the target cell is a neuronal cell (i.e., the vector targets neuronal cells). However, in particular embodiments, the target cell is a non-neuronal cell (i.e., the vector does not target neuronal cells). In some embodiments, the target cell is a glial cell, Muller cell, and/or retinal pigment epithelial (RPE) cell. The promoter may be derived from any species, including human. In one embodiment, the promoter is “cell specific.” The term “cell-specific” or that a promoter is “specific for” a cell type means that the particular promoter selected for the recombinant vector can direct expression of the selected transgene in a particular cell or ocular cell type. In one embodiment, the promoter is specific for expression of the transgene in photoreceptor cells. In another embodiment, the promoter is specific for expression in the rods and/or cones. In another embodiment, the promoter is specific for expression of the transgene in RPE cells. In another embodiment, the promoter is specific for expression of the transgene in ganglion cells. In another embodiment, the promoter is specific for expression of the transgene in Muller cells. In another embodiment, the promoter is specific for expression of the transgene in bipolar cells. In another embodiment, the promoter is specific for expression of the transgene in ON-bipolar cells. In one embodiment, the promoter is metabotropic glutamate receptor 6 (mGluR6) promoter (see, Vardi et al, mGluR6 Transcripts in Non-neuronal Tissues, J Histochem Cytochem. 2011 December; 59(12): 1076-1086, which is incorporated herein by reference). In another embodiment, the promoter is an enhancer-linked mGluR6 promoter. In another embodiment, the promoter is specific for expression of the transgene in OFF-bipolar cells. In another embodiment, the promoter is specific for expression of the transgene in horizontal cells. In another embodiment, the promoter is specific for expression of the transgene in amacrine cells. In another embodiment, the transgene is expressed in any of the above noted ocular cells. In another embodiment, the promoter is the human G-protein-coupled receptor protein kinase 1 (GRK1) promoter (Genbank Accession number AY327580). In another embodiment, the promoter is the human interphotoreceptor retinoid-binding protein proximal (IRBP) promoter.

In some embodiments, the promoter is of a small size, e.g., under 1000 bp, due to the size limitations of the AAV vector. In some embodiments, the promoter is less than 1000, 900, 800, 700, 600, 500, 400 or 300 bp in size. In particular embodiments, the promoter is under 400 bp. In some embodiments, the promoter is a promoter selected from the CRALBP (RLBP), EFla, HSP70, AAT1, ALB, PCK1, CAG, RPE65, MECP, or sCBA promoter. In some embodiments, the promoter comprises a nucleotide sequence having at least 85%, at least 90%, at least 95%, at least 96%, at least 97%, at least 98%, at least 99%, or 100% identity of any one of SEQ ID NOs: 6, 8, 12, 14, 16, 18, 20, 31, 32, or 36 or codon-optimized and/or fragment thereof. In some embodiments, the promoter comprises a nucleotide sequence having at least 85%, at least 90%, at least 95%, at least 96%, at least 97%, at least 98%, at least 99%, or 100% identity of SEQ ID NO: 6 or codon-optimized and/or fragment thereof. In some embodiments, the promoter comprises a nucleotide sequence having at least 85%, at least 90%, at least 95%, at least 96%, at least 97%, at least 98%, at least 99%, or 100% identity of SEQ ID NO: 8 or codon-optimized and/or fragment thereof. In some embodiments, the promoter comprises a nucleotide sequence having at least 85%, at least 90%, at least 95%, at least 96%, at least 97%, at least 98%, at least 99%, or 100% identity of SEQ ID NO: 12 or codon-optimized and/or fragment thereof. In some embodiments, the promoter comprises a nucleotide sequence having at least 85%, at least 90%, at least 95%, at least 96%, at least 97%, at least 98%, at least 99%, or 100% identity of SEQ ID NO: 14 or codon-optimized and/or fragment thereof. In some embodiments, the promoter comprises a nucleotide sequence having at least 85%, at least 90%, at least 95%, at least 96%, at least 97%, at least 98%, at least 99%, or 100% identity of SEQ ID NO: 16 or codon-optimized and/or fragment thereof. In some embodiments, the promoter comprises a nucleotide sequence having at least 85%, at least 90%, at least 95%, at least 96%, at least 97%, at least 98%, at least 99%, or 100% identity of SEQ ID NO: 18 or codon-optimized and/or fragment thereof. In some embodiments, the promoter comprises a nucleotide sequence having at least 85%, at least 90%, at least 95%, at least 96%, at least 97%, at least 98%, at least 99%, or 100% identity of SEQ ID NO: 20 or codon-optimized and/or fragment thereof. In some embodiments, the promoter comprises a nucleotide sequence having at least 85%, at least 90%, at least 95%, at least 96%, at least 97%, at least 98%, at least 99%, or 100% identity of SEQ ID NO: 31 or codon-optimized and/or fragment thereof. In some embodiments, the promoter comprises a nucleotide sequence having at least 85%, at least 90%, at least 95%, at least 96%, at least 97%, at least 98%, at least 99%, or 100% identity of SEQ ID NO: 32 or codon-optimized and/or fragment thereof. In some embodiments, the promoter comprises a nucleotide sequence having at least 85%, at least 90%, at least 95%, at least 96%, at least 97%, at least 98%, at least 99%, or 100% identity of SEQ ID NO: 36 or codon-optimized and/or fragment thereof. In some embodiments, the promoter comprises the nucleotide sequence of any one of SEQ ID NOs: 6, 8, 12, 14, 16, 18, 20, 31, 32, or 36 or codon-optimized and/or fragment thereof. In some embodiments, the promoter is associated with strong expression in the eye. In some embodiments, the promoter is a CRALBP or RPE65 promoter (e.g., a promoter having the nucleotide sequence of SEQ ID NO: 6 or 32). In some embodiments, the promoter is associated with strong expression in the liver. In some embodiments, the promoter is an AAT1, ALB, or PCK1 promoter (e.g., a promoter having the nucleotide sequence of SEQ ID NO: 16, 18, or 20, respectively). In some embodiments, if the gene to be expressed in the AAV vector is CFH or CFHL (e.g., a gene comprising the nucleotide sequence of any one of SEQ ID NOs: 1-3 or 5, or a codon-optimized and/or fragment thereof), then the promoter is less than 1000, less than 900, less than 800, less than 700, less than 600, less than 500, less than 400, or less than 300 bp in size. In some embodiments, if the gene to be expressed in the AAV vector is CFH or CFHL (e.g., a gene comprising the nucleotide sequence of any one of SEQ ID NOs: 1-3 or 5, or a codon-optimized and/or fragment thereof), then the promoter is a promoter selected from the CRALBP, EF1a, HSP70 or sCBA promoter. In some embodiments, if the gene to be expressed in the AAV vector is CFH or CFHL (e.g., a gene comprising the nucleotide sequence of any one of SEQ ID NOs: 1-3 or 5, or a codon-optimized and/or fragment thereof), then the promoter comprises the nucleotide sequence of any one of SEQ ID NOs: 6, 8, 12, 14, 16, 18, 20, 31, 32, or 36 or codon-optimized and/or fragments thereof. In some embodiments, any of the promoters disclosed herein is coupled with a viral intron (e.g., an SV40i intron).

In another embodiment, the promoter is the native CFH promoter. Useful promoters include, without limitation, the rod opsin promoter, the red-green opsin promoter, the blue opsin promoter, the cGMP-β-phosphodiesterase promoter, the mouse opsin promoter (Beltran et al 2010 cited above), the rhodopsin promoter (Mussolino et al, Gene Ther, July 2011, 18(7):637-45); the alpha-subunit of cone transducin (Morrissey et al, BMC Dev, Biol, January 2011, 11:3); beta phosphodiesterase (PDE) promoter; the retinitis pigmentosa (RP1) promoter (Nicoud et al, J. Gene Med, December 2007, 9(12): 1015-23); the NXNL2/NXNL1 promoter (Lambard et al, PLoS One, October 2010, 5(10):e13025), the RPE65 promoter; the retinal degeneration slow/peripherin 2 (Rds/perph2) promoter (Cai et al, Exp Eye Res. 2010 August; 91(2): 186-94); and the VMD2 promoter (Kachi et al, Human Gene Therapy, 2009 (20:31-9)). Each of these documents is incorporated by reference herein. In one embodiment, the promoter is of a small size, under 1000 bp, due to the size limitations of the AAV vector. In another embodiment, the promoter is under 400 bp.

In certain embodiments, any promoters suitable for use in AAV vectors may be used with the vectors of the disclosure. Examples of suitable promoters include constitutive promoters such as a CMV promoter (optionally with the CMV enhancer), RSV promoter (optionally with the RSV enhancer), SV40 promoter, MoMLV promoter, CB promoter, the dihydrofolate reductase promoter, the chicken 3-actin (CBA) promoter, CBA/CAG promoter, and the immediate early CMV enhancer coupled with the CBA promoter, or a EFla promoter, etc. In some embodiments a cell- or tissue-specific promoter is utilized (e.g., a rod, cone, or ganglia derived promoter). In certain embodiments, the promoter is small enough to be compatible with the disclosed constructs, e.g., the CB promoter. In some embodiments, the promoter is a constitutive promoter. In another embodiment, the promoter is cell-specific. The term “cell-specific” means that the particular promoter selected for the recombinant vector can direct expression of the selected transgene in a particular ocular cell type. In one embodiment, the promoter is specific for expression of the transgene in photoreceptor cells. In another embodiment, the promoter is specific for expression in the rods and cones. In another embodiment, the promoter is specific for expression in the rods. In another embodiment, the promoter is specific for expression in the cones. In another embodiment, the promoter is specific for expression of the transgene in RPE cells. In another embodiment, the transgene is expressed in any of the above noted ocular cells.

Other useful promoters include transcription factor promoters including, without limitation, promoters for the neural retina leucine zipper (Nrl), photoreceptor-specific nuclear receptor Nr2e3, and basic-leucine zipper (bZIP). In one embodiment, the promoter is of a small size, under 1000 bp, due to the size limitations of the AAV vector. In another embodiment, the promoter is under 400 bp.

Other regulatory sequences useful herein include enhancer sequences. Enhancer sequences useful herein include the IRBP enhancer (Nicoud 2007, cited above), immediate early cytomegalovirus enhancer, one derived from an immunoglobulin gene or SV40 enhancer, the cis-acting element identified in the mouse proximal promoter, etc.

Selection of these and other common vector and regulatory elements are conventional and many such sequences are available. See, e.g., Sambrook et al, and references cited therein at, for example, pages 3.18-3.26 and 16.17-16.27 and Ausubel et al., Current Protocols in Molecular Biology, John Wiley & Sons, New York, 1989). It is understood that not all vectors and expression control sequences will function equally well to express all of the transgenes as described herein. However, one of skill in the art may make a selection among these, and other, expression control sequences to generate the rAAV vectors of the disclosure.

Production of rAAV Vectors

Numerous methods are known in the art for production of rAAV vectors, including transfection, stable cell line production, and infectious hybrid virus production systems which include adenovirus-AAV hybrids, herpesvirus-AAV hybrids (Conway, J E et al., (1997). Virology 71(11):8780-8789) and baculovirus-AAV hybrids. In some embodiments, rAAV production cultures for the production of rAAV virus particles comprise; 1) suitable host cells, including, for example, human-derived cell lines such as HeLa or A549 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a transgene (such as a transgene encoding a complement system polypeptide (e.g. CFH, FHL1, FHR1, FHR2, FHR3, FHR4, or FHR5) or a biologically active fragment thereof) flanked by at least one AAV ITR sequence; and 5) suitable media and media components to support rAAV production. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors.

The rAAV particles can be produced using methods known in the art. See, e.g., U.S. Pat. Nos. 6,566,118; 6,989,264; and 6,995,006. In practicing the disclosure, host cells for producing rAAV particles include mammalian cells, insect cells, plant cells, microorganisms and yeast. Host cells can also be packaging cells in which the AAV rep and cap genes are stably maintained in the host cell or producer cells in which the AAV vector genome is stably maintained. Exemplary packaging and producer cells are derived from A549 or HeLa cells. AAV vectors are purified and formulated using standard techniques known in the art.

Recombinant AAV particles are generated by transfecting producer cells with a plasmid (cis-plasmid) containing a rAAV genome comprising a transgene flanked by the 145 nucleotide-long AAV ITRs and a separate construct expressing the AAV rep and CAP genes in trans. In addition, adenovirus helper factors such as E1A, E1B, E2A, E40RF6 and VA RNAs, etc. may be provided by either adenovirus infection or by transfecting a third plasmid providing adenovirus helper genes into the producer cells. Packaging cell lines suitable for producing adeno-associated viral vectors may be readily accomplished given readily available techniques (see e.g., U.S. Pat. No. 5,872,005). The helper factors provided will vary depending on the producer cells used and whether the producer cells already carry some of these helper factors.

In some embodiments, rAAV particles may be produced by a triple transfection method, such as the exemplary triple transfection method provided infra. Briefly, a plasmid containing a rep gene and a capsid gene, along with a helper adenoviral plasmid, may be transfected (e.g., using the calcium phosphate method) into a cell line, and virus may be collected and optionally purified.

In some embodiments, rAAV particles may be produced by a producer cell line method, such as the exemplary producer cell line method provided infra (see also (referenced in Martin et al., (2013) Human Gene Therapy Methods 24:253-269). Briefly, a cell line (e.g., a HeLa cell line) may be stably transfected with a plasmid containing a rep gene, a capsid gene, and a promoter-transgene sequence. Cell lines may be screened to select a lead clone for rAAV production, which may then be expanded to a production bioreactor and infected with an adenovirus (e.g., a wild-type adenovirus) as helper to initiate rAAV production. Virus may subsequently be harvested, adenovirus may be inactivated (e.g., by heat) and/or removed, and the rAAV particles may be purified.

In some aspects, a method is provided for producing any rAAV particle as disclosed herein comprising (a) culturing a host cell under a condition that rAAV particles are produced, wherein the host cell comprises (i) one or more AAV package genes, wherein each said AAV packaging gene encodes an AAV replication and/or encapsidation protein; (ii) a rAAV pro-vector comprising a nucleic acid encoding a therapeutic polypeptide and/or nucleic acid as described herein flanked by at least one AAV ITR, and (iii) an AAV helper function; and (b) recovering the rAAV particles produced by the host cell. In some embodiments, said at least one AAV ITR is selected from the group consisting of AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV or the like. In some embodiments, the encapsidation protein is an AAV2 encapsidation protein. In some embodiments, the encapsidation protein is an AAV.7m8 encapsidation protein.

Suitable rAAV production culture media of the present disclosure may be supplemented with serum or serum-derived recombinant proteins at a level of 0.5-20 (v/v or w/v). Alternatively, as is known in the art, rAAV vectors may be produced in serum-free conditions which may also be referred to as media with no animal-derived products. One of ordinary skill in the art may appreciate that commercial or custom media designed to support production of rAAV vectors may also be supplemented with one or more cell culture components know in the art, including without limitation glucose, vitamins, amino acids, and or growth factors, in order to increase the titer of rAAV in production cultures.

rAAV production cultures can be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa, and SF-9 cells which can be cultured in a variety of ways including, for example, spinner flasks, stirred tank bioreactors, and disposable systems such as the Wave bag system.

rAAV vector particles of the disclosure may be harvested from rAAV production cultures by lysis of the host cells of the production culture or by harvest of the spent media from the production culture, provided the cells are cultured under conditions known in the art to cause release of rAAV particles into the media from intact cells, as described more fully in U.S. Pat. No. 6,566,118). Suitable methods of lysing cells are also known in the art and include for example multiple freeze/thaw cycles, sonication, microfluidization, and treatment with chemicals, such as detergents and/or proteases.

In a further embodiment, the rAAV particles are purified. The term “purified” as used herein includes a preparation of rAAV particles devoid of at least some of the other components that may also be present where the rAAV particles naturally occur or are initially prepared from. Thus, for example, isolated rAAV particles may be prepared using a purification technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like.

In some embodiments, the rAAV production culture harvest is clarified to remove host cell debris. In some embodiments, the production culture harvest is clarified by filtration through a series of depth filters including, for example, a grade DOHC Millipore Millistak+HC Pod Filter, a grade A1HC Millipore Millistak+HC Pod Filter, and a 0.2 μm Filter Opticap XL 10 Millipore Express SHC Hydrophilic Membrane filter. Clarification can also be achieved by a variety of other standard techniques known in the art, such as, centrifugation or filtration through any cellulose acetate filter of 0.2 m or greater pore size known in the art.

In some embodiments, the rAAV production culture harvest is further treated with Benzonase® to digest any high molecular weight DNA present in the production culture. In some embodiments, the Benzonase® digestion is performed under standard conditions known in the art including, for example, a final concentration of 1-2.5 units/mL of Benzonase® at a temperature ranging from ambient to 37° C. for a period of 30 minutes to several hours.

rAAV particles may be isolated or purified using one or more of the following purification steps: equilibrium centrifugation; flow-through anionic exchange filtration; tangential flow filtration (TFF) for concentrating the rAAV particles; rAAV capture by apatite chromatography; heat inactivation of helper virus; rAAV capture by hydrophobic interaction chromatography; buffer exchange by size exclusion chromatography (SEC); nanofiltration; and rAAV capture by anionic exchange chromatography, cationic exchange chromatography, or affinity chromatography. These steps may be used alone, in various combinations, or in different orders. Methods to purify rAAV particles are found, for example, in Xiao et al., (1998) Journal of Virology 72:2224-2232; U.S. Pat. Nos. 6,989,264 and 8,137,948; and WO 2010/148143.

In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 7, or a biologically active fragment of thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 9, or a biologically active fragment thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 11, or a biologically active fragment thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 13, or a biologically active fragment thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 15, or a biologically active fragment thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 17, or a biologically active fragment thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 19, or a biologically active fragment thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 21, or a biologically active fragment thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 22, or a biologically active fragment thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 23, or a biologically active fragment thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 24, or a biologically active fragment thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 25, or a biologically active fragment thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 26, or a biologically active fragment thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 27, or a biologically active fragment thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 28, or a biologically active fragment thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 29, or a biologically active fragment thereof. In some embodiments, any of the vectors disclosed herein comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of SEQ ID NO: 30, or a biologically active fragment thereof.

### Pharmaceutical Compositions

Also provided herein for use in any of the methods disclosed herein are pharmaceutical compositions comprising an rAAV particle comprising a transgene encoding a CFH polypeptide or a biologically active fragment and/or variant thereof and/or therapeutic nucleic acid, and a pharmaceutically acceptable carrier. The pharmaceutical compositions may be suitable for any mode of administration described herein; for example, by intravitreal administration.

In some embodiments, the composition comprises a polypeptide (or a nucleic acid encoding a polypeptide) that processes (e.g., cleaves) the complement system polypeptide encoded by the transgene in the rAAV. However, in particular embodiments, the composition does not comprise a polypeptide (or a nucleic acid encoding a polypeptide) that processes (e.g., cleaves) the complement system polypeptide encoded by the transgene in the rAAV.

Gene therapy protocols for retinal diseases, such as leber congenital amaurosis (LCA), retinitis pigmentosa, and age-related macular degeneration require the localized delivery of the vector to the cells in the retina. The cells that will be the treatment target in these diseases are either the photoreceptor cells in the retina or the cells of the RPE underlying the neurosensory retina. Delivering gene therapy vectors to these cells requires injection into the subretinal space between the retina and the RPE. In some embodiments, the disclosure provides methods to deliver rAAV gene therapy vectors encoding a CFH polypeptide or a biologically active fragment and/or variant thereof to cells of the retina.

In some embodiments, the pharmaceutical compositions comprising a rAAV described herein and a pharmaceutically acceptable carrier is suitable for administration to a human subject. Such carriers are well known in the art (see, e.g., Remington's Pharmaceutical Sciences, 15th Edition, pp. 1035-1038 and 1570-1580). In some embodiments, the pharmaceutical compositions comprising a rAAV described herein and a pharmaceutically acceptable carrier is suitable for ocular injection. In some embodiments, the pharmaceutical composition is suitable for intravitreal injection. In some embodiments, the pharmaceutical composition is suitable for subretinal delivery. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms. The compositions are generally formulated as sterile and substantially isotonic solution.

In one embodiment, the recombinant AAV containing the desired transgene encoding a CFH polypeptide or a biologically active fragment and/or variant thereof and constitutive or tissue or cell-specific promoter for use in the target ocular cells as detailed above is formulated into a pharmaceutical composition intended for subretinal or intravitreal injection. In some embodiments, the compositions disclosed herein targets cells of any one or more regions of the macula including, for example, the umbo, the foveolar, the foveal avascular zone, the fovea, the parafovea, or the perifovea. Such formulation involves the use of a pharmaceutically and/or physiologically acceptable vehicle or carrier, particularly one suitable for administration to the eye, e.g., by subretinal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20. In another embodiment, the pharmaceutically acceptable carrier comprises a surfactant, such as perfluorooctane (Perfluoron liquid).

In certain embodiments of the methods described herein, the pharmaceutical composition described above is administered to the subject by subretinal injection. In other embodiments, the pharmaceutical composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Routes of administration may be combined, if desired. In certain embodiments, the pharmaceutical compositions of the disclosure are administered after administration of an initial loading dose of the complement system protein.

In some embodiments, any of the vectors/pharmaceutical compositions disclosed herein are administered to a patient such that they target glial cells, Muller cells, and/or retinal pigment epithelial cells. In some embodiments, the route of administration does not specifically target neurons. In some embodiments, the route of administration is chosen such that it reduces the risk of retinal detachment in the patient (e.g., intravitreal rather than subretinal administration). In some embodiments, intravitreal administration is chosen if the vector/composition is to be administered to an elderly adult (e.g., at least 60 years of age). In particular embodiments, any of the vectors/pharmaceutical compositions disclosed herein are administered to a subject intravitreally. Procedures for intravitreal injection are known in the art (see, e.g., Peyman, G. A., et al. (2009) Retina 29(7):875-912 and Fagan, X. J. and Al-Qureshi, S. (2013) Clin. Experiment. Ophthalmol. 41(5):500-7). Briefly, a subject for intravitreal injection may be prepared for the procedure by pupillary dilation, sterilization of the eye, and administration of anesthetic. Any suitable mydriatic agent known in the art may be used for pupillary dilation. Adequate pupillary dilation may be confirmed before treatment. Sterilization may be achieved by applying a sterilizing eye treatment, e.g., an iodide-containing solution such as Povidone-Iodine (BETADINE®). A similar solution may also be used to clean the eyelid, eyelashes, and any other nearby tissues (e.g., skin). Any suitable anesthetic may be used, such as lidocaine or proparacaine, at any suitable concentration. Anesthetic may be administered by any method known in the art, including without limitation topical drops, gels or jellies, and subconjuctival application of anesthetic. Prior to injection, a sterilized eyelid speculum may be used to clear the eyelashes from the area. The site of the injection may be marked with a syringe. The site of the injection may be chosen based on the lens of the patient. For example, the injection site may be 3-3.5 mm from the limbus in pseudophakic or aphakic patients, and 3.5-4 mm from the limbus in phakic patients. The patient may look in a direction opposite the injection site. During injection, the needle may be inserted perpendicular to the sclera and pointed to the center of the eye. The needle may be inserted such that the tip ends in the vitreous, rather than the subretinal space. Any suitable volume known in the art for injection may be used. After injection, the eye may be treated with a sterilizing agent such as an antiobiotic. The eye may also be rinsed to remove excess sterilizing agent.

Furthermore, in certain embodiments it is desirable to perform non-invasive retinal imaging and functional studies to identify areas of specific ocular cells to be targeted for therapy. In these embodiments, clinical diagnostic tests are employed to determine the precise location(s) for one or more subretinal injection(s). These tests may include ophthalmoscopy, electroretinography (ERG) (particularly the b-wave measurement), perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, etc.

These, and other desirable tests, are described in International Patent Application No. PCT/US2013/022628. In view of the imaging and functional studies, in some embodiments, one or more injections are performed in the same eye in order to target different areas of retained bipolar cells. The volume and viral titer of each injection is determined individually, as further described below, and may be the same or different from other injections performed in the same, or contralateral, eye. In another embodiment, a single, larger volume injection is made in order to treat the entire eye. In one embodiment, the volume and concentration of the rAAV composition is selected so that only a specific region of ocular cells is impacted. In another embodiment, the volume and/or concentration of the rAAV composition is a greater amount, in order reach larger portions of the eye, including non-damaged ocular cells.

The composition may be delivered in a volume of from about 0.1 μL to about 1 mL, including all numbers within the range, depending on the size of the area to be treated, the viral titer used, the route of administration, and the desired effect of the method. In some embodiments, the volume is between 25-100 μL. In some embodiments, the volume is between 40-60 μL. In one embodiment, the volume is about 50 μL. In another embodiment, the volume is about 70 μL. In a preferred embodiment, the volume is about 100 μL. In another embodiment, the volume is about 125 μL. In another embodiment, the volume is about 150 μL. In another embodiment, the volume is about 175 μL. In yet another embodiment, the volume is about 200 L. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 L. In another embodiment, the volume is about 1000 μL. An effective concentration of a recombinant adeno-associated virus carrying a nucleic acid sequence encoding the desired transgene under the control of the cell-specific promoter sequence desirably ranges from about 10⁷ and 10¹³ vector genomes per milliliter (vg/mL) (also called genome copies/mL (GC/mL)). The rAAV infectious units are measured as described in S. K. McLaughlin et al, 1988 J. Virol., 62: 1963, which is incorporated herein by reference. Preferably, the concentration in the retina is from about 1.5×10⁹ vg/mL to about 1.5×10¹² vg/mL, and more preferably from about 1.5×10⁹ vg/mL to about 1.5×10¹¹ vg/mL. In certain preferred embodiments, the effective concentration is about 2.5×10¹⁰ vg to about 1.4×10¹¹. In one embodiment, the effective concentration is about 1.4×10⁸ vg/mL. In one embodiment, the effective concentration is about 3.5×10¹⁰ vg/mL. In another embodiment, the effective concentration is about 5.6×10¹¹ vg/mL. In another embodiment, the effective concentration is about 5.3×10¹² vg/mL. In yet another embodiment, the effective concentration is about 1.5×10¹² vg/mL. In another embodiment, the effective concentration is about 1.5×10¹³ vg/mL. In one embodiment, the effective dosage (total genome copies delivered) is from about 10⁷ to 10¹³ vector genomes. It is desirable that the lowest effective concentration of virus be utilized in order to reduce the risk of undesirable effects, such as toxicity, retinal dysplasia and detachment. Still other dosages and administration volumes in these ranges may be selected by the attending physician, taking into account the physical state of the subject, preferably human, being treated, the age of the subject, the particular ocular disorder and the degree to which the disorder, if progressive, has developed. For extra-ocular delivery, the dosage will be increased according to the scale-up from the retina. Intravenous delivery, for example may require doses on the order of 1.5×10¹³ vg/kg.

Pharmaceutical compositions useful in the methods of the disclosure are further described in PCT publication No. WO2015168666 and PCT publication no. WO2014011210, the contents of which are incorporated by reference herein.

### Methods of Treatment/Prophylaxis

In some embodiments, the disclosure provides a method for treating a subject having a disease or disorder, wherein the subject has one or more CFH mutations. A subject “has” a CFH mutation if DNA from a sample (e.g., a blood sample or a sample from the patient's eye) from the subject is determined to carry one or more CFH mutations. In some embodiments, any of the methods disclosed herein are for treating a subject in whom it has been determined has one or more CFH mutations. In some embodiments, the presence or absence of any of the CFH mutations disclosed herein is determined by genetic testing.

Described herein are various methods of preventing, treating, arresting progression of or ameliorating the ocular disorders and retinal changes associated therewith. Generally, the methods include administering to a mammalian subject in need thereof, an effective amount of a composition comprising a recombinant adeno-associated virus (AAV) described above, carrying a transgene encoding a CFH polypeptide or a biologically active fragment and/or variant thereof under the control of regulatory sequences which express the product of the gene in the subject's ocular cells, and a pharmaceutically acceptable carrier. Any of the AAV described herein are useful in the methods described below.

Gene therapy protocols for retinal diseases, such as LCA, retinitis pigmentosa, and age-related macular degeneration require the localized delivery of the vector to the cells in the retina. The cells that will be the treatment target in these diseases are either the photoreceptor cells in the retina or the cells of the RPE underlying the neurosensory retina. Delivering gene therapy vectors to these cells requires injection into the subretinal space between the retina and the RPE. In some embodiments, the disclosure provides methods to deliver rAAV gene therapy vectors comprising a complement system gene or a fragment thereof to cells of the retina.

In a certain aspect, the disclosure provides a method of treating a subject having age-related macular degeneration (AMD), comprising the step of administering to the subject any of the vectors of the disclosure. In certain embodiments, the vectors are administered at a dose between 2.5×10¹⁰ vg and 1.4×10¹¹ vg/per eye in about 50 μL to about 100 μL. In certain embodiments, the vectors are administered at a dose between 1.0×10¹¹ vg and 1.5×10¹³ vg/per eye in about 50 μL to about 100 μL. In certain embodiments, the vectors are administered at a dose between 1.0×10¹¹ vg and 1.5×10¹² vg/per eye in about 50 μL to about 100 μL. In certain embodiments, the vectors are administered at a dose of about 1.4×10¹² vg/per eye in about 50 μl to about 100 μl. In certain embodiments, the vectors are administered at a dose of 1.4×10¹² vg/per eye in about 50 μL to about 100 μL. In certain embodiments, the pharmaceutical compositions of the disclosure comprise a pharmaceutically acceptable carrier. In certain embodiments, the pharmaceutical compositions of the disclosure comprise PBS. In certain embodiments, the pharmaceutical compositions of the disclosure comprise pluronic. In certain embodiments, the pharmaceutical compositions of the disclosure comprise PBS, NaCl and pluronic. In certain embodiments, the vectors are administered by intravitreal injection in a solution of PBS with additional NaCl and pluronic.

In some embodiments, any of the vectors of the present disclosure used according to the methods disclosed herein is capable of inducing at least 5%, at least 10%, at least 20%, at least 50%, at least 100%, at least 150%, at least 200%, at least 250%, at least 300%, at least 400%, at least 500%, at least 700%, at least 900%, at least 1000%, at least 1100%, at least 1500%, or at least 2000% expression of CFH and/or FHL1 in a target cell disclosed herein (e.g., an RPE or liver cell) as compared to the endogenous expression of CFH and/or FHL1 in the target cell. In some embodiments, expression of any of the vectors disclosed herein in a target cell disclosed herein (e.g., an RPE or liver cell) results in at least 5%, at least 10%, at least 20%, at least 50%, at least 100%, at least 150%, at least 200%, at least 250%, at least 300%, at least 400%, at least 500%, at least 700%, at least 900%, at least 1000%, at least 1100%, at least 1500%, or at least 2000% levels of CFH and/or FHL1 activity in the target cell as compared to endogenous levels of CFH and/or FHL1 activity in the target cell.

In some embodiments, any of the vectors disclosed herein is administered to cell(s) or tissue(s) in a test subject. In some embodiments, the cell(s) or tissue(s) in the test subject express less CFH and/or FHL1, or less functional CFH and/or FHL1, than expressed in the same cell type or tissue type in a reference control subject or population of reference control subjects. In some embodiments, the reference control subject or population of reference control subjects does not have any of the CFH mutations disclosed herein. In some embodiments, the reference control subject is of the same age and/or sex as the test subject. In some embodiments, the reference control subject is a healthy subject, e.g., the subject does not have a disease or disorder of the eye. In some embodiments, the reference control subject does not have a disease or disorder of the eye associated with activation of the complement cascade. In some embodiments, the reference control subject does not have macular degeneration. In some embodiments, the eye and/or a specific cell type of the eye (e.g., cells in the foveal region) in the test subject express at least 95%, at least 90%, at least 80%, at least 70%, at least 60%, at least 50%, at least 40%, at least 30%, at least 20%, at least 10%, at least 5%, or at least 1% less CFH and/or FHL1 or functional CFH and/or FHL1 as compared to the levels in the reference control subject or population of reference control subjects. In some embodiments, a the eye or a specific cell type of the eye (e.g., cells in the foveal region) in the test subject express CFH and/or FHL1 protein having any of the CFH and/or FHL1 mutations disclosed herein. In some embodiments, the eye or a specific cell type of the eye (e.g., cells in the foveal region) in the reference control subject do not express a CFH and/or FHL1 protein having any of the CFH and/or FHL1 mutations disclosed herein. In some embodiments, expression of any of the vectors disclosed herein in the cell(s) or tissue(s) of the test subject results in an increase in levels of CFH and/or FHL1 protein or functional CFH and/or FHL1 protein. In some embodiments, expression of any of the vectors disclosed herein in the cell(s) or tissue(s) of the test subject results in an increase in levels of CFH and/or FHL1 protein or functional CFH and/or FHL1 protein such that the increased levels are within 90%, 80%, 70%, 60%, 50%, 40%, 30%, 20%, 10%, 5%, or 1% of, or are the same as, the levels of CFH and/or FHL1 protein or functional CFH and/or FHL1 protein expressed by the same cell type or tissue type in the reference control subject or population of reference control subjects. In some embodiments, expression of any of the vectors disclosed herein in the cell(s) or tissue(s) of the test subject results in an increase in levels of CFH and/or FHL1 protein or functional CFH and/or FHL1 protein, but the increased levels of CFH and/or FHL1 protein or functional CFH and/or FHL1 protein do not exceed the levels of CFH and/or FHL1 protein or functional CFH and/or FHL1 protein expressed by the same cell type or tissue type in the reference control subject or population of reference control subjects. In some embodiments, expression of any of the vectors disclosed herein in the cell(s) or tissue(s) of the test subject results in an increase in levels of CFH and/or FHL1 protein or functional CFH and/or FHL1 protein, but the increased levels of CFH and/or FHL1 protein or functional CFH and/or FHL1 protein exceed the levels of CFH and/or FHL1 protein or functional CFH and/or FHL1 protein by no more than 1%, no more than 5%, no more than 10%, no more than 20%, no more than 25%, no more than 30%, no more than 40%, no more than 50%, no more than 60%, no more than 70%, no more than 80%, no more than 90%, or 100% of the levels expressed by the same cell type or tissue type in the reference control subject or population of reference control subjects. In some embodiments, any of the treatment and/or prophylactic methods disclosed herein are applied to a subject. In some embodiments, the subject is a mammal. In some embodiments, the subject is a human. In some embodiments, the human is an adult. In some embodiments, the human is an elderly adult. In some embodiments, the human is at least 40, at least 45, at least 50, at least 55, at least 60, at least 65, at least 70, at least 75, at least 80, at least 85, at least 90, or at least 95 years of age. In particular embodiments, the human is at least 60 or 65 years of age.

In some embodiments, any of the treatment and/or prophylactic methods disclosed herein is for use in treatment of a patient having one or more CFH mutations. In some embodiments, any of the treatment and/or prophylactic methods disclosed herein is for use in treatment of a patient having one or more CFH mutations that causes macular degeneration (AMD) or that increases the likelihood that a patient develops AMD. In some embodiments, any of the treatment and/or prophylactic methods disclosed herein is for use in treatment of a patient having one or more mutations that causes atypical hemolytic uremic syndrome (aHUS) or that increases the likelihood that a patient develops aHUS. In some embodiments, the subject has a loss-of-function mutation in the subject's CFH gene.

In some embodiments, any of the treatment and/or prophylactic methods disclosed herein is for use in treatment of a patient having one or more mutations in the patient's CFH gene. In some embodiments, the treatment and/or prophylactic method is for use in treating a patient in whom it has been determined has one or more of any of the CFH mutations disclosed herein. In some embodiments, the patient has a mutation in one or more of CCP domains 1-20, or any combination thereof. In some embodiments, the patient has a mutation in one or more of CCP domains 1-2 or 18-20. In some embodiments, the patient has a mutation in CCP1. In some embodiments, the patient has a mutation in CCP2. In some embodiments, the patient has a mutation in CCP3. In some embodiments, the patient has a mutation in CCP4. In some embodiments, the patient has a mutation in CCP5. In some embodiments, the patient has a mutation in CCP6. In some embodiments, the patient has a mutation in CCP7. In some embodiments, the patient has a mutation in CCP8. In some embodiments, the patient has a mutation in CCP9. In some embodiments, the patient has a mutation in CCP10. In some embodiments, the patient has a mutation in CCP11. In some embodiments, the patient has a mutation in CCP12. In some embodiments, the patient has a mutation in CCP13. In some embodiments, the patient has a mutation in CCP14. In some embodiments, the patient has a mutation in CCP15. In some embodiments, the patient has a mutation in CCP16. In some embodiments, the patient has a mutation in CCP17. In some embodiments, the patient has a mutation in CCP18. In some embodiments, the patient has a mutation in CCP19. In some embodiments, the patient has a mutation in CCP20. In some embodiments, the patient has one or more mutations in the disulphide bond sites in the CFH protein. In some embodiments, the mutation is one or more of the mutations selected from the group consisting of: H402Y, G69E, D194N, W314C, A806T, Q950H, p.Ilel84fsX, p.Lys204fsX, c.1697-17_-8del, A161S, A173G, R175Q, V62I, V1007L, S890I, S193L, I216T, A30lNfs*25 (i.e., amino acid A at position 301 changed to amino acid N by a frameshifting mutation, which also leads to translation termination 25 residues downstream), A301N, W379R, Q400K, Q950H, T956M, R1210C, N1050Y, E936D, Q408X, R1078S, c.350+6T->G, R567G, R53C, R53H, R2T, A892V, R567G, I221V, S159N, P562H, F960S, R303W, R303Q, K666N, G1194D, P258L, G650V, D130N, S58A, R166W, R232Q, R127H, K1202N, G397Stop, Stop450R, R830W, I622L, T732M, S884Y, L24V, Y235H, K527N, R582H, C973Y, V1089M, E123G, T291S, R567K, E625Stop, N802S, N1056K, R1203W, Q1076E, P26S, T46A, T91S, C129Y, R166Q, E167Q, R175P, C192F, W198*, V206M, G218*, M239T, Y277*, C325Y, R341H, R364L, P384R, C431S, D454A, A473V, P503A, N516K, I551T, H699R, F717L, W978R, P981S, A1010V, W1037*, P1051L, I1059T, Q1143E, R1206H, T1227I, L24V, H169R, R257H, K410E, V609I, D619N, A892V, G1002R, G278S, T30*, I32Stop, R78G, Q81P, V111E, W134R, P139S, M162V, E189Stop, K224Del, K224Del, A307A, H332Y, S411T, C448Y, L479Stop, R518T, T519A, C536R, C564P, C569Stop, L578Stop, P621T, C623S, C630W, E635D, K670T, Q672Q, C673Y, C673S, S714Stop, S722*, C733Y, V737V, E762Stop, N774Stop, R780I, G786*, M823T, V835L, E847V, E850K, C853R, C853T, C864S, C870R, H878H, I881L, E889Stop, H893R, Y899Stop, Y899D, C915S, C915Stop, W920R, Q925Stop, C926F, Y951H, C959Y, P968*, I970V, T987A, N997T, G1011*, T1017I, Y1021F, C1043R, T1046T, V1054I, V1060A, V1060L, C1077W, T1097W, T1097T, D1119G, D1119N, P1130L, V1134G, E1135R, E1137L, E1139Stop, Y1142D, Y1142C, C1152S, W1157R, P1161T, C1163T, P1166L, V1168E, V1168Stop, I1169L, E1172Stop, Y1177C, R1182S, W1183L, W1183R, W1183L, W1183Stop, W1183C, T1184R, T1184A, K1186H, K1188De1, L1189R, L1189F, S1191L, S1191W, E1195Stop, V1197A, E1198A, E1198Stop, F1199S, V1200L, G1204E, L1207R, S1211P, R1215Q, R1215G, T1216De1, C1218R, Y1225*, P1226S, L3V, H821Y, E954de1, G255E, T1038R, V383A, V641A, P213A, I221V, E229K, R2T, R1072G, G967E, N819S, V579F, G19K, A18S, K834E, T504M, R662I, P668L, G133R, I184T, L697F, H1165Y, G1110A, pIle808_Gln809del, I760L, T447R, I808M, I868M, L765F, N767S, R567G, K768N, S209L, Q628K, D214Y, N401D, I216K, Q464R, I777V, E229D, M823I, R232Ter, S266L, P260S, E23G, C80Y, R78T, R582H, N638D, N638S, P258L, L3F, R257H, G240R, G69R, D855N, M11I, K472N, Q840H, E850K, Y899H, T645M, M805V, K919T, E201G, V407A, I907L, T914K, H332R, V144M, S652G, D195N, C146S, P661R, E677Q, V482I, T34R, A421T, R281G, C509Y, K666N, P440S, C442G, N607D, A425V, G667E, P440L, I49V, R387G, E625K, E625Ter, T135S, P43S, K283E, I124V, T36V, I563T, G350E, D619G, T321I, T286A, P384L, T739N, M515L, V158A, G727R, T724K, F717L, M162V, C178R, G700R, A161T, F176L, R295S, F298Y, G297S, P300L, R1040K, V552L, T310I, T531A, G928D, Ter386RextTer 69a€, Q1143K, Y534C, P981L, K308N, D538E, R1215Ter, E105V, T1017I, N1050I, P935S, Y951H, T1097M, D947H, E961D, G962S, G964E, I970V, R1072T, P1114L, S1122T, F960C, R1074C, R1182T, R1074L, S884Y, S890T, V837I, V941F, V158I, D748V, I216T, H371N, L750F, P418T, M432V, D693N, A746E, V111E, c.2237-2A>G, P982S, V579A, E591D, V579I, V65I, P418S, Y1067C, D772N, V72L, E189K, A1027P, D798N, N61D, P384S, N521S, P1068S, E395K, N774S, H577R, E833K, K6E, H337R, R444C, L741F, Y42F, D288E, S705F, R1040G, D214H, N757D, I861M, G848E, P923S, E201K, E902A, R303Q, G366E, D538H, K82R, E721K, Y1008H, R1074P, A806S, Q807R, C389Y, H764Y, K867N, P392T, L394M, E456K, F459L, Y398C, E570K, D214N, I574V, I574T, G631C, T880I, V865F, V576A, N776S, P633S, N22D, P634A, N822I, R885S, R232L, E635D, R778K, L827V, C267R, Y779C, R582C, L77S, R257C, Y327H, N75K, L74F, S836T, Y243H, c.1519+5_1519+8delGT . . . , K507Q, A892S, I15T, P924L, A14V, N842K, G894R, G894E, Y271C, C9W, T504R, V683M, L385Pheaf, S898R, Q408H, G409S, T34K, E648G, I412V, E338D, P799S, G480E, D798E, D195Y, R341C, D485H, D485G, K598Q, Y420H, P599T, N434H, R441T, C431G, V149A, V349I, T679A, P43T, G45D, R662G, T519I, L121P, P364L, P621A, H373Y, D538MfsTer14, H371P, T544A, T131A, R166G, V177I, V177A, R729S, F717V, N718S, S991G, L98I, Y1016Ter, T1217del, M1001T, K1004E, A1010T, G1011D, T1017A, T1031A, L1125F, R1203G, L1214M, W1096DfsTer20, H939N F960L, D966H, M1064I, E1071K, N1095K, T1106A, G1107E, C1109W, P1111S, V1197I, Y1075F, S1079N, P1080S, E1082G, or Stol232. In particular embodiments, the mutation is one or more of the mutations selected from the group consisting of: R2T, L3V, R53C, R53H, S58A, G69E, D90G, R175Q, S193L, I216T, I221V, R303W, H402Y, Q408X, P503A, G650V, R1078S, and R1210C. As used in the context of mutations, asterisk refers to a stop codon resulting in a C-terminal truncation. In some embodiments, any of the CFH mutant amino acid positions described herein correspond to the amino acid CFH sequence of SEQ ID NO: 33. In some embodiments, any of the CFH mutant amino acid positions described herein correspond to the amino acid CFH sequence of SEQ ID NO: 37. In some embodiments, any of the CFH mutant amino acid positions described herein correspond to the amino acid CFH sequence of SEQ ID NO: 38.

In some embodiments, the patient has any one of the following CFH mutations: R2T, L3V, R53C, R53H, S58A, D90G, D130N, R175Q, R175P, I221V, R303W, R303Q, Q400K, Y402H, P503A, R567G, G650V, S890I, T956M, G1194D, or R1210C. In some embodiments, the patient has any one or more of the following CFH mutations: R2T, R53C, R53H, S58A, D130N, R175Q, R175P, 1221V, R303W, R303Q, P503A, R567G, G650V, G1194D, or R1210C. In some embodiments, the patient has an R2T mutation. In some embodiments, the patient has an L3V mutation. In some embodiments, the patient has an R53C mutation. In some embodiments, the patient has an R53H mutation. In some embodiments, the patient has an S58A mutation. In some embodiments, the patient has a D90G mutation. In some embodiments, the patient has a D130N mutation. In some embodiments, the patient has an R175Q mutation. In some embodiments, the patient has an R175P mutation. In some embodiments, the patient has an I221V mutation. In some embodiments, the patient has an R303W mutation. In some embodiments, the patient has an R303Q mutation. In some embodiments, the patient has a Q400K mutation. In some embodiments, the patient has a Y402H mutation. In some embodiments, the patient has a P503A mutation. In some embodiments, the patient has an R567G mutation. In some embodiments, the patient has a G650V mutation. In some embodiments, the patient has an S890I mutation. In some embodiments, the patient has a T956M mutation. In some embodiments, the patient has a G1194D mutation. In some embodiments, the patient has an R1210C mutation. In some embodiments, any of the CFH mutant amino acid positions described herein correspond to the amino acid CFH sequence of SEQ ID NO: 33. In some embodiments, any of the CFH mutant amino acid positions described herein correspond to the amino acid CFH sequence of SEQ ID NO: 37. In some embodiments, any of the CFH mutant amino acid positions described herein correspond to the amino acid CFH sequence of SEQ ID NO: 38.

In some embodiments, the subject has one or more mutations in other complement pathway genes, optionally in combination with CFH mutations. It is understood that polymorphism is present in some complement pathway genes, resulting in multiple variants of the same gene. As used herein, the term “mutation” refers to a genetic variant or an amino acid sequence encoded by the genetic variant, even if the genetic variant is recognized as wildtype in certain populations. In some embodiments, the subject has one or more C3 mutations. In some embodiments, the subject has one or more C3 mutations selected from R102G, K155Q, V619M, and R735W. In some embodiments, the subject has one or more complement factor B (CFB) mutations. In some embodiments, the subject has the I242L mutation of CFB. In some embodiments, the subject has the 32R mutation of CFB. In some embodiments, the subject has the CFH 62V, C3 102G, and/or CFB 32R mutations. In some embodiments, the subject is homozygous for CFH 62V, C3 102G, and CFB 32R, which combination of homozygous mutations is predicted to have a prevalence of about 1.4% in the Caucasian population and is enriched in the AMD patient population.

In some embodiments, the patient is homozygous for any of the mutations disclosed herein. In some embodiments, the patient is heterozygous for any of the mutations disclosed herein. In particular embodiments, the patient expresses a mutant CFH protein, wherein the mutant CFH protein has reduced CFH activity as compared to a wildtype CFH protein (e.g., a CFH protein having the amino acid sequence of any one of SEQ ID NOs: 33, 34, 37 or 38). In some embodiments, the CFH activity is the ability to bind to C3b. In some embodiments, the CFH activity is the ability to act as a cofactor with CFI and facilitate C3b cleavage. In some embodiments, the CFH activity is the ability to bind to a cell surface (e.g., an erythrocyte and/or endothelial cell). In some embodiments, the CFH activity is the ability to bind to heparin. In some embodiments, the CFH activity is the ability to inhibit C5b9 levels as a result of complement activation, e.g., as measured in a Wieslab AP assay (see, e.g., Example 1). In some embodiments, the CFH activity is the ability to inhibit hemolysis. In some embodiments, if the mutant CFH protein were tested in a functional assay, the mutant CFH protein would display reduced CFH activity as compared to a wildtype CFH protein (e.g., a CFH protein having the amino acid sequence of any one of SEQ ID NOs: 33, 34, 37 or 38). Examples of CFH mutants associated with reduced CFH activity include R2T, R53C, R53H, S58A, D130N, R175Q, R175P, I221V, R303W, R303Q, P503A, R567G, G650V, G1194D, or R1210C CFH mutants. See, e.g., the functional assays in Example 1. In some embodiments, if a cell expresses less of a variant CFH polypeptide than the same cell would express a wildtype control CFH polypeptide, then the variant CFH is determined to be functionally impaired.

In some embodiments, if the subject has a CFH mutation that is associated with reduced CFH activity, then a greater amount of a vector encoding a CFH polypeptide (or biologically active fragment and/or variant thereof) is administered to the subject than if the subject did not have a CFH mutation associated with reduced CFH activity in one or more activity assays. Examples of CFH mutants that do not have reduced activity in a cell expression assay, a C3b affinity assay, a decay acceleration assay, a cofactor assay, a hemolysis assay, or a Wieslab AP assay include L3V, D90G, Q400K, Y402H, S890I, or T956M CFH mutants. See, e.g., Example 1.

In some embodiments, any of the methods disclosed herein are for treating a subject in whom it has been determined has one or more of any of the mutations disclosed herein.

In some embodiments, any of the vectors disclosed herein are for use in treating a renal disease or complication. In some embodiments, the renal disease or complication is associated with AMD in the patient. In some embodiments, the renal disease or complication is associated with aHUS in the patient. In some embodiments, the vector administered for treating a renal disease or complication comprises a promoter that is associated with strong expression in the liver. In some embodiments, the promoter is an AAT1, PCK1, or ALB1 promoter (e.g., a promoter comprising the nucleotide sequence of any one of SEQ ID Nos: 16, 18 or 20).

The retinal diseases described above are associated with various retinal changes. These may include a loss of photoreceptor structure or function; thinning or thickening of the outer nuclear layer (ONL); thinning or thickening of the outer plexiform layer (OPL); disorganization followed by loss of rod and cone outer segments; shortening of the rod and cone inner segments; retraction of bipolar cell dendrites; thinning or thickening of the inner retinal layers including inner nuclear layer, inner plexiform layer, ganglion cell layer and nerve fiber layer; opsin mislocalization; overexpression of neurofilaments; thinning of specific portions of the retina (such as the fovea or macula); loss of ERG function; loss of visual acuity and contrast sensitivity; loss of optokinetic reflexes; loss of the pupillary light reflex; and loss of visually guided behavior. In one embodiment, a method of preventing, arresting progression of or ameliorating any of the retinal changes associated with these retinal diseases is provided. As a result, the subject's vision is improved, or vision loss is arrested and/or ameliorated.

In a particular embodiment, a method of preventing, arresting progression of or ameliorating vision loss associated with an ocular disorder in the subject is provided. Vision loss associated with an ocular disorder refers to any decrease in peripheral vision, central (reading) vision, night vision, day vision, loss of color perception, loss of contrast sensitivity, or reduction in visual acuity.

In another embodiment, a method of targeting one or more type(s) of ocular cells for gene augmentation therapy in a subject in need thereof is provided. In another embodiment, a method of targeting one or more type of ocular cells for gene suppression therapy in a subject in need thereof is provided. In yet another embodiment, a method of targeting one or more type of ocular cells for gene knockdown/augmentation therapy in a subject in need thereof is provided. In another embodiment, a method of targeting one or more type of ocular cells for gene correction therapy in a subject in need thereof is provided. In still another embodiment, a method of targeting one or more type of ocular cells for neurotropic factor gene therapy in a subject in need thereof is provided.

In any of the methods described herein, the targeted cell may be an ocular cell. In one embodiment, the targeted cell is a glial cell. In one embodiment, the targeted cell is an RPE cell. In another embodiment, the targeted cell is a photoreceptor. In another embodiment, the photoreceptor is a cone cell. In another embodiment, the targeted cell is a Muller cell. In another embodiment, the targeted cell is a bipolar cell. In yet another embodiment, the targeted cell is a horizontal cell. In another embodiment, the targeted cell is an amacrine cell. In still another embodiment, the targeted cell is a ganglion cell. In still another embodiment, the gene may be expressed and delivered to an intracellular organelle, such as a mitochondrion or a lysosome.

As used herein “photoreceptor function loss” means a decrease in photoreceptor function as compared to a normal, non-diseased eye or the same eye at an earlier time point. As used herein, “increase photoreceptor function” means to improve the function of the photoreceptors or increase the number or percentage of functional photoreceptors as compared to a diseased eye (having the same ocular disease), the same eye at an earlier time point, a non-treated portion of the same eye, or the contralateral eye of the same patient. Photoreceptor function may be assessed using the functional studies described above and in the examples below, e.g., ERG or perimetry, which are conventional in the art.

For each of the described methods, the treatment may be used to prevent the occurrence of retinal damage or to rescue eyes having mild or advanced disease. As used herein, the term “rescue” means to prevent progression of the disease to total blindness, prevent spread of damage to uninjured ocular cells, improve damage in injured ocular cells, or to provide enhanced vision. In one embodiment, the composition is administered before the disease becomes symptomatic or prior to photoreceptor loss. By symptomatic is meant onset of any of the various retinal changes described above or vision loss. In another embodiment, the composition is administered after disease becomes symptomatic. In yet another embodiment, the composition is administered after initiation of photoreceptor loss. In another embodiment, the composition is administered after outer nuclear layer (ONL) degeneration begins. In some embodiments, it is desirable that the composition is administered while bipolar cell circuitry to ganglion cells and optic nerve remains intact.

In another embodiment, the composition is administered after initiation of photoreceptor loss. In yet another embodiment, the composition is administered when less than 90% of the photoreceptors are functioning or remaining, as compared to a non-diseased eye. In another embodiment, the composition is administered when less than 80% of the photoreceptors are functioning or remaining. In another embodiment, the composition is administered when less than 70% of the photoreceptors are functioning or remaining. In another embodiment, the composition is administered when less than 60% of the photoreceptors are functioning or remaining. In another embodiment, the composition is administered when less than 50% of the photoreceptors are functioning or remaining. In another embodiment, the composition is administered when less than 40% of the photoreceptors are functioning or remaining. In another embodiment, the composition is administered when less than 30% of the photoreceptors are functioning or remaining. In another embodiment, the composition is administered when less than 20% of the photoreceptors are functioning or remaining. In another embodiment, the composition is administered when less than 10% of the photoreceptors are functioning or remaining. In one embodiment, the composition is administered only to one or more regions of the eye. In another embodiment, the composition is administered to the entire eye.

In another embodiment, the method includes performing functional and imaging studies to determine the efficacy of the treatment. These studies include ERG and in vivo retinal imaging, as described in the examples below. In addition visual field studies, perimetry and microperimetry, pupillometry, mobility testing, visual acuity, contrast sensitivity, color vision testing may be performed.

In yet another embodiment, any of the above described methods is performed in combination with another, or secondary, therapy. The therapy may be any now known, or as yet unknown, therapy which helps prevent, arrest or ameliorate any of the described retinal changes and/or vision loss. In one embodiment, the secondary therapy is encapsulated cell therapy (such as that delivering Ciliary Neurotrophic Factor (CNTF)). See, Sieving, P. A. et al, 2006. Proc Natl Acad Sci USA, 103(10):3896-3901, which is hereby incorporated by reference. In another embodiment, the secondary therapy is a neurotrophic factor therapy (such as pigment epithelium-derived factor, PEDF; ciliary neurotrophic factor 3; rod-derived cone viability factor (RdCVF) or glial-derived neurotrophic factor). In another embodiment, the secondary therapy is anti-apoptosis therapy (such as that delivering X-linked inhibitor of apoptosis, XIAP). In yet another embodiment, the secondary therapy is rod derived cone viability factor 2. The secondary therapy can be administered before, concurrent with, or after administration of the rAAV described above.

In some embodiments, any of the vectors or compositions disclosed herein is administered to a subject in combination with any of the other vectors or compositions disclosed herein. In some embodiments, any of the vectors or compositions disclosed herein is administered to a subject in combination with another therapeutic agent or therapeutic procedure. In some embodiments, the additional therapeutic agent is an anti-VEGF therapeutic agent (e.g., such as an anti-VEGF antibody or fragment thereof such as ranibizumab, bevacizumab or aflibercept), a vitamin or mineral (e.g., vitamin C, vitamin E, lutein, zeaxanthin, zinc or copper), omega-3 fatty acids, and/or Visudyne™. In some embodiments, the other therapeutic procedure is a diet having reduced omega-6 fatty acids, laser surgery, laser photocoagulation, submacular surgery, retinal translocation, and/or photodynamic therapy.

In some embodiments, any of the vectors disclosed herein is administered to a subject in combination with an additional agent needed for processing and/or improving the function of the protein encoded by the vector/composition. For example, if the vector comprises a CFH gene, the vector may be administered to a patient in combination with an antibody (or a vector encoding that antibody) that potentiates the activity of the expressed CFH protein. Examples of such antibodies are found in WO2016/028150 or WO2019139481, which are each incorporated herein in their entirety.

### Kits

In some embodiments, any of the vectors disclosed herein is assembled into a pharmaceutical or diagnostic or research kit to facilitate their use in therapeutic, diagnostic or research applications. A kit may include one or more containers housing any of the vectors disclosed herein and instructions for use. In some embodiments, the kit includes instructions for administering any of the vectors or compositions disclosed herein to a subject in whom it has been determined has one or more of any of the CFH mutations disclosed herein.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

### EXAMPLES

The disclosure now being generally described, it will be more readily understood by reference to the following examples, which are included merely for purposes of illustration of certain embodiments and embodiments of the present disclosure, and are not intended to limit the disclosure.

### Example 1: CFH Mutant Analysis

This example was designed to assess the impact of certain CFH mutations on the activity of CFH. Briefly, various CFH mutants were generated and tested in different functional assays. Assays are discussed below.

### Fluorescent CFI Cofactor Assay

Recombinant human CFH variants were serially diluted in Tris-buffered saline (1XTBS; 50 mM Tris, 150 mM NaCl, pH 7.4) at concentrations ranging from 0.006 mg/mL to 0.5 mg/mL in a twelve point dose response. Assay components were added to opaque half-area black polystyrene plates in the following order in a 50 μL final reaction volume: 0.02 mg purified human C3b, 5 μM ANS, CFH in final concentrations from 1.2 μg/mL to 100 μg/mL and 0.1 μg of CFI. Reactions were mixed briefly by shaking at 4000 revolutions per minute and read over 30 minutes at 30 seconds intervals at 30° C. Fluorescence readings were recorded in kinetic mode with excitation set to 386 nm and emission set to 472 nm. Negative controls for reaction rate included no C3b, no CFI, and no CFH. All reagents, instruments, and laboratory supplies used in this experiment are listed in Table 1 and 2.

TABLE 1
Equipment and supplies for fluorescent CFI cofactor assay

| Equipment | Manufacturer |
|---|---|
| SpectraMax M5e | Molecular Devices |
| Pipette (2 μL) | VistaLab |
| Pipette (20 μL) | VistaLab |
| Pipette (200 μL) | VistaLab |
| Pipette (1000 μL) | VistaLab |
| Plate shaker | Lab-Line Instruments |
| 96 well half area opaque polystyrene plates | Costar |
| Prism 7 for Windows | GraphPad |

TABLE 2
Reagents for fluorescent CFI cofactor assay

| Reagent | Manufacturer | Concentration | Storage |
|---|---|---|---|
| 8-anilinonaphthalene- | Arcos | 100 mM in methyl | −20° C. |
| 1 sulfonic acid, CAS# | | sulfoxide; dilute 1:2000 | |
| 82-76-8 | | with 1X TBS | |
| Methyl sulfoxide, | Arcos | n/a | ambient |
| molecular biology grade; | | | |
| Cas#67-68-5 | | | |
| 10X Tris-buffered saline | Boston | Dilute to 1X with water | ambient |
| (TBS); 500 mM Tris, | Biomedical | | |
| 1.5M NaCl, pH 7.4 | | | |
| Complement factor 3b | Complement | 1.03 mg/mL | −80° C. |
| | Technology | | |
| Complement factor 1 | Complement | 1.04 mg/mL; dilute to | −80° C. |
| | Technology | 0.01 mg/mL in 1X TBS | |
| rhCFH variant (155 | Thermo | Various concentrations; | 4° C. |
| kDa) | | dilute with 1X TBS | |
| | | between range 0.006 | |
| | | mg/mL to 0.5 mg/mL | |

Abbreviations:
kDa, kilodaltons

The kinetic plots were analyzed by assessment of the slope in the linear range (typically between 200 and 900 seconds). The reaction rates, i.e., the slopes of observed reduction in fluorescence at 472 nm (corresponding to C3b cleavage), were calculated for each CFH concentration, carried out in triplicate. The inverse of the average reaction rate plus or minus the standard deviation were plotted for each concentration of CFH in pg/mL on a log scale. The data were fit with a 4-point sigmoidal curve to calculate the EC₅₀ value. The ratio of the EC₅₀ for each CFH Variant over the EC₅₀ for VYE was calculated.

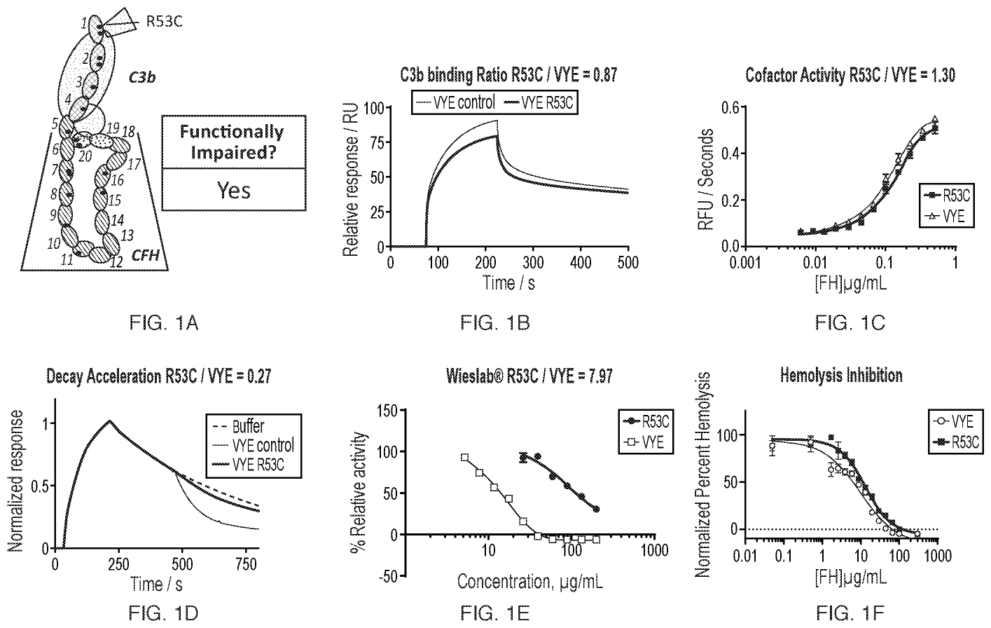

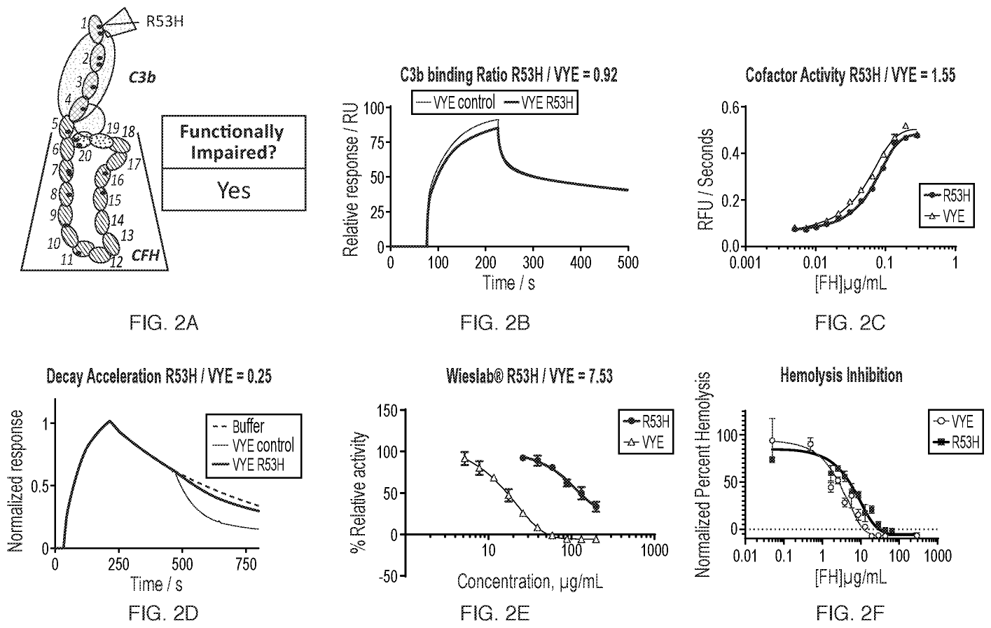

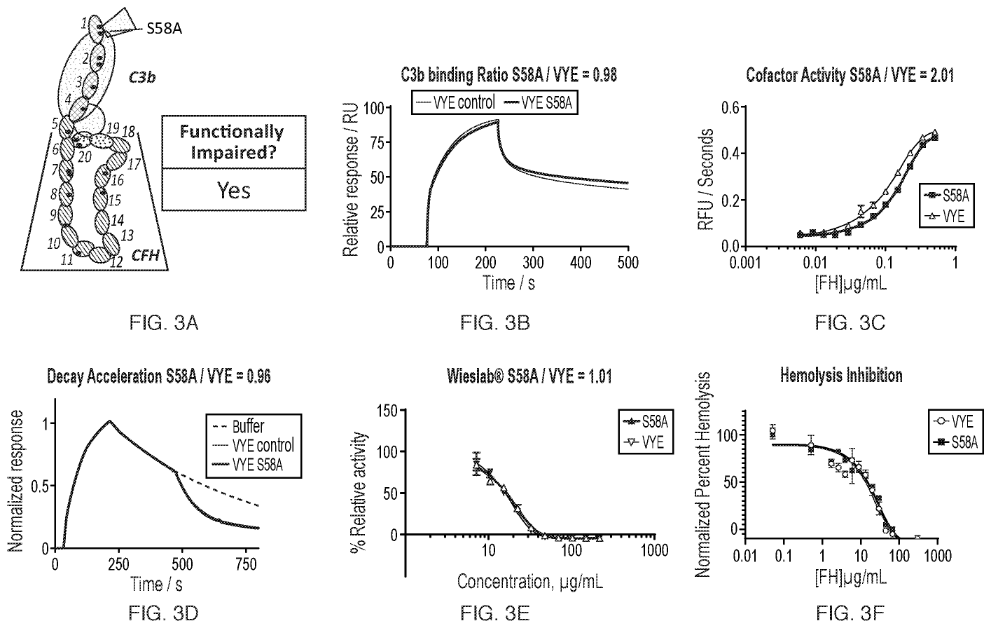

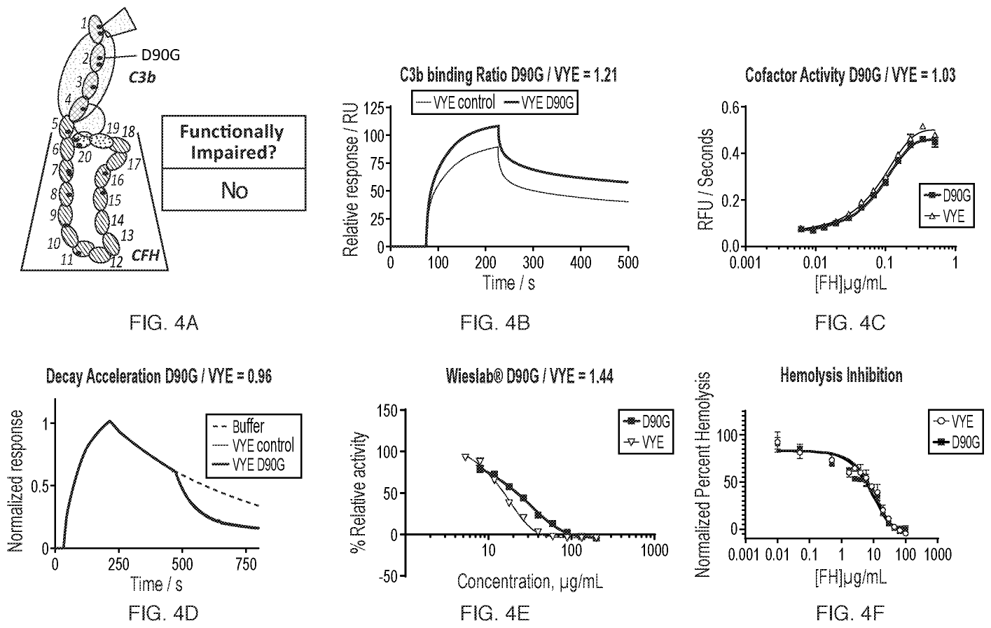

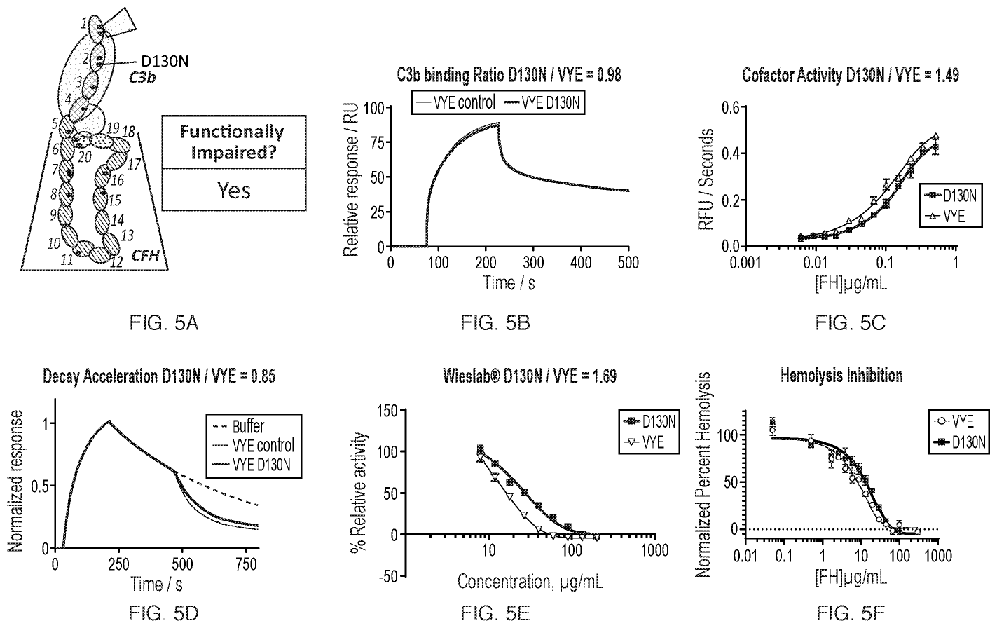

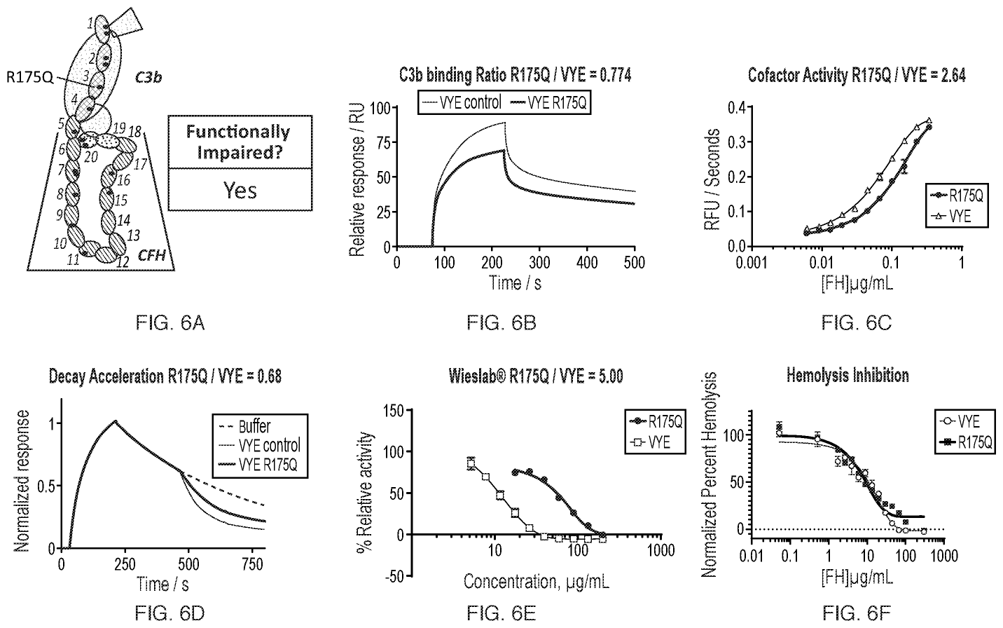

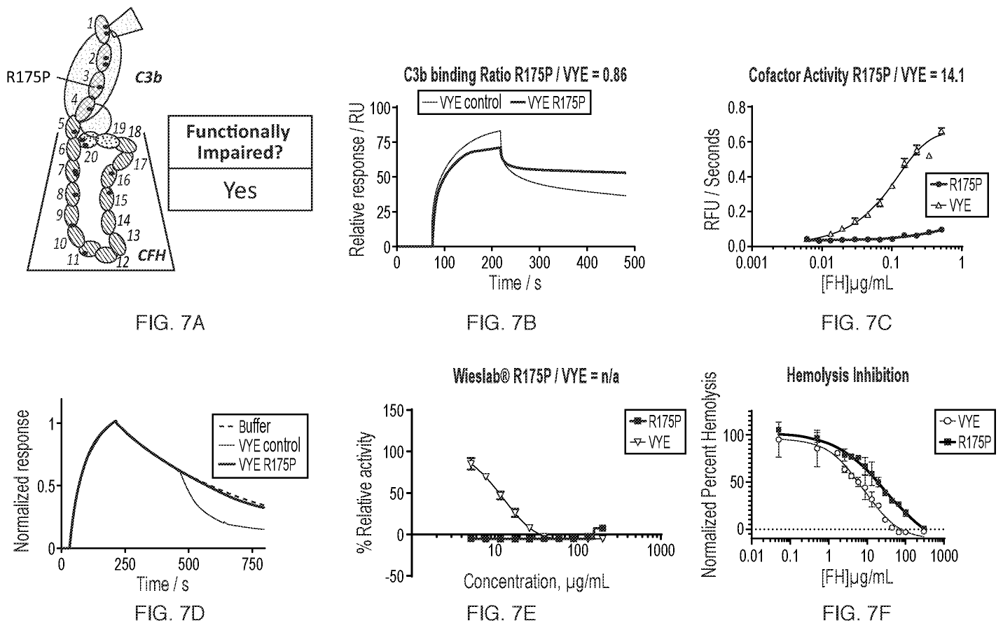

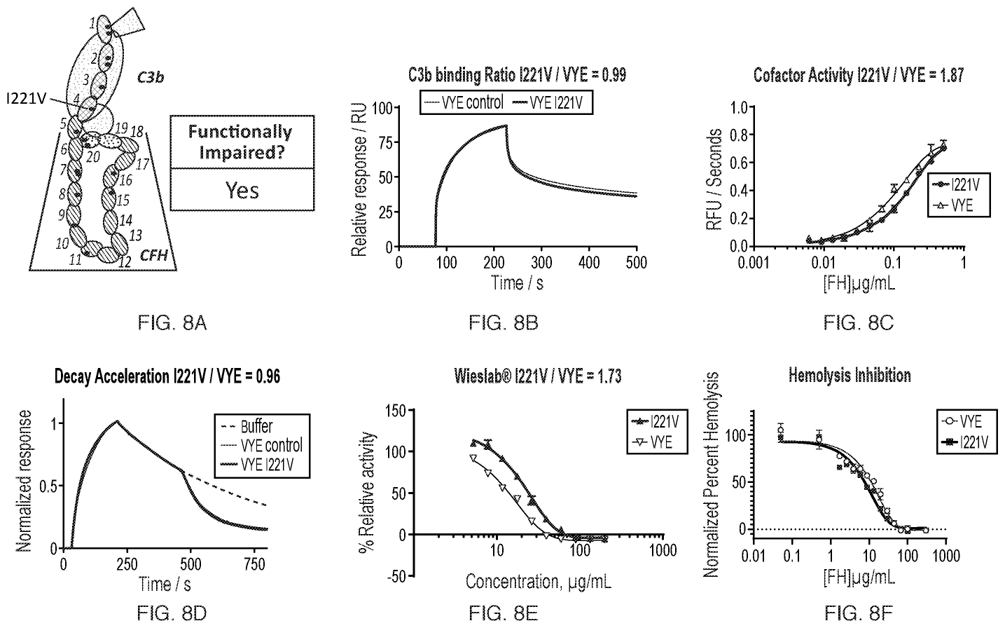

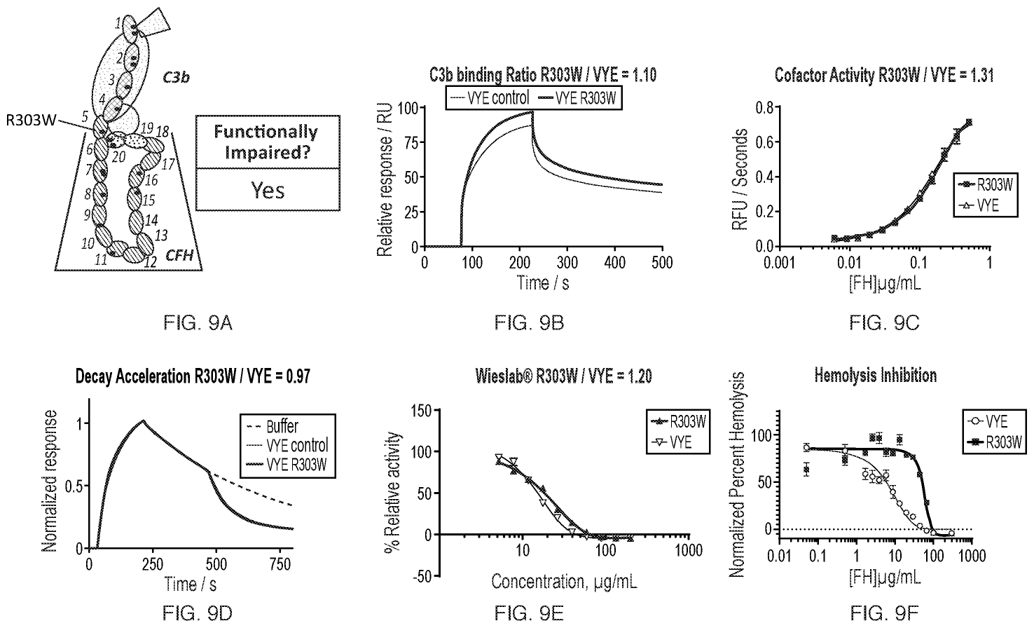

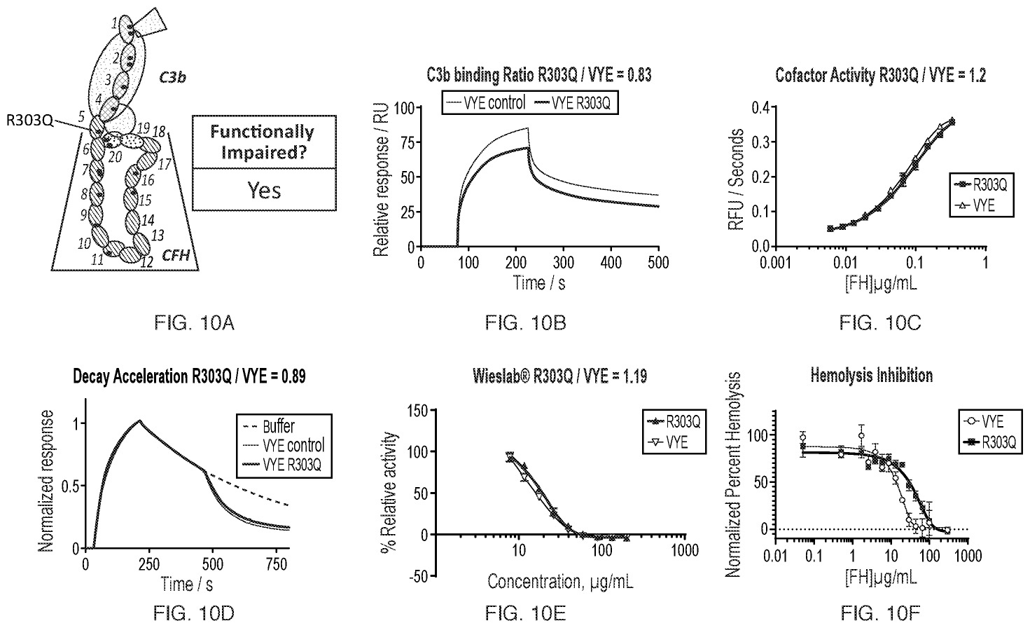

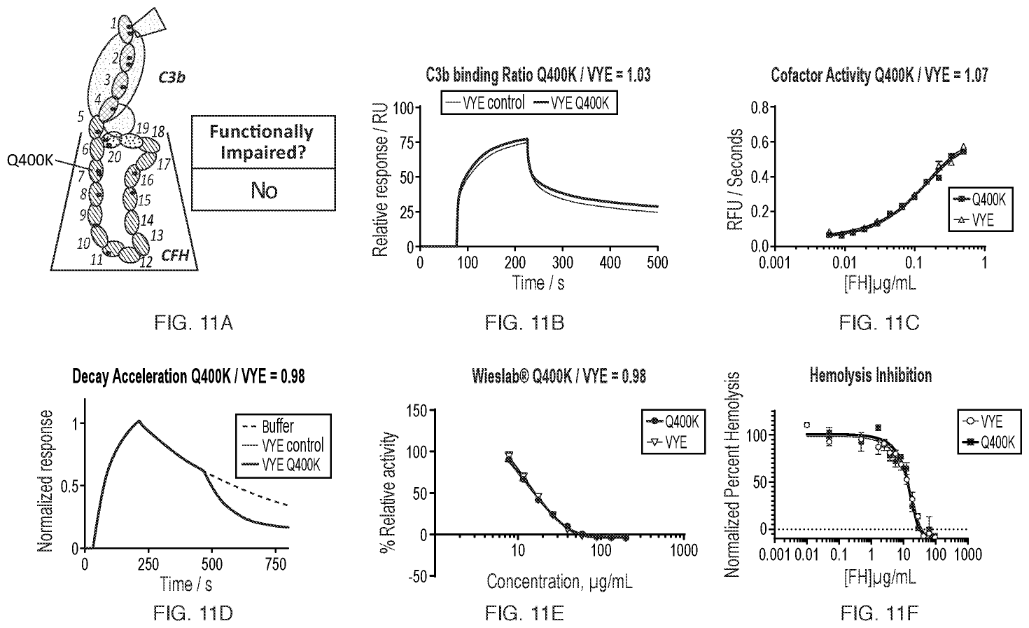

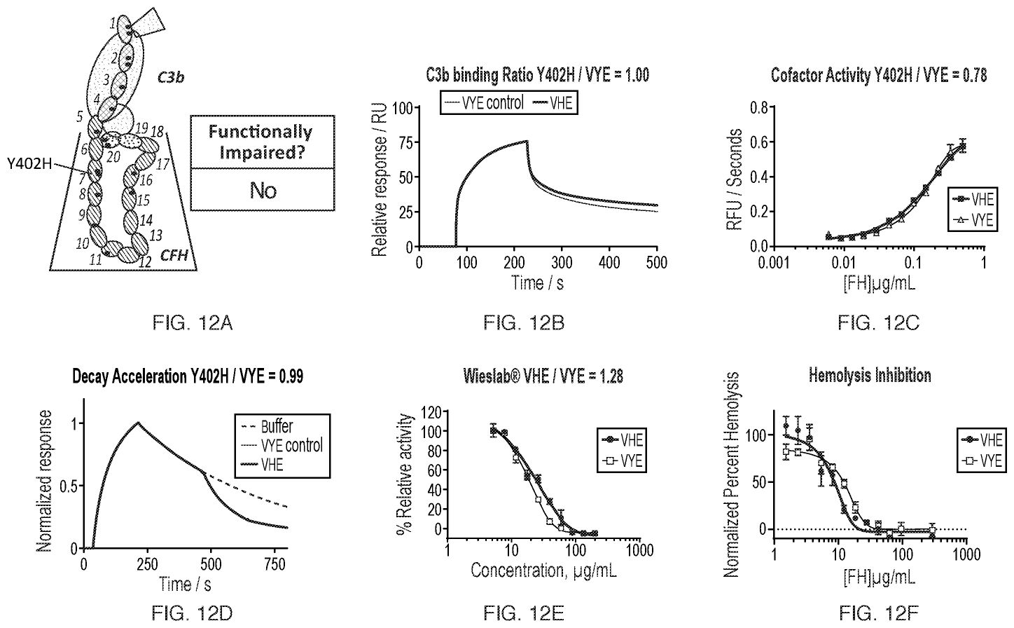

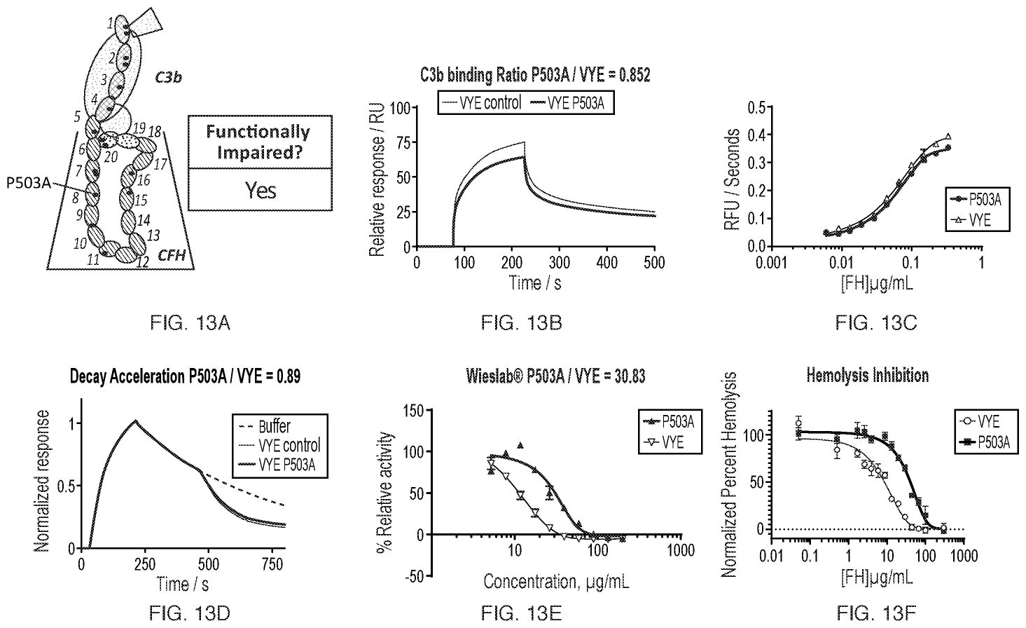

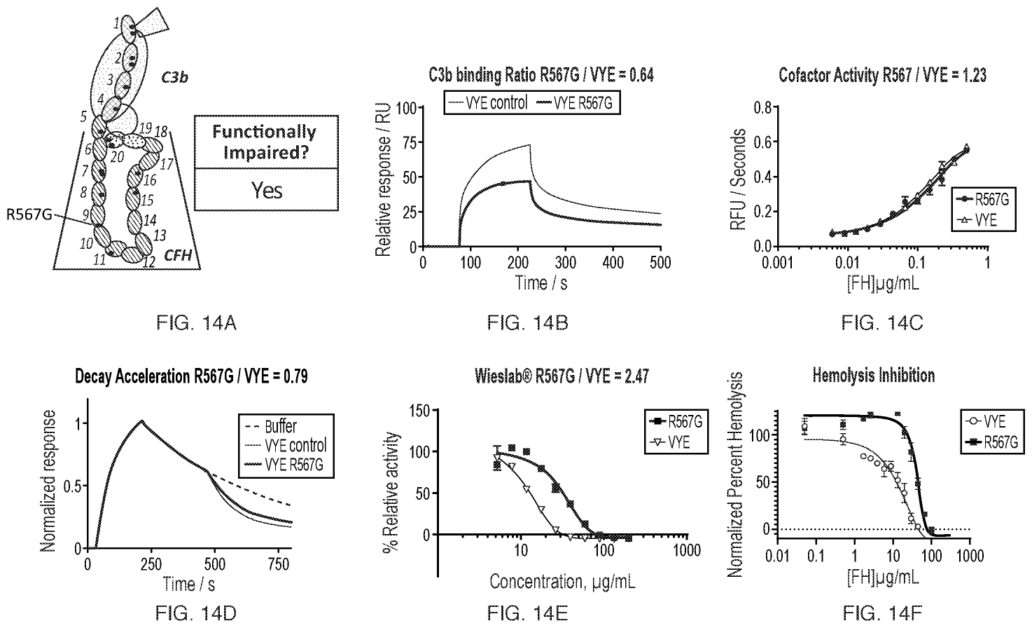

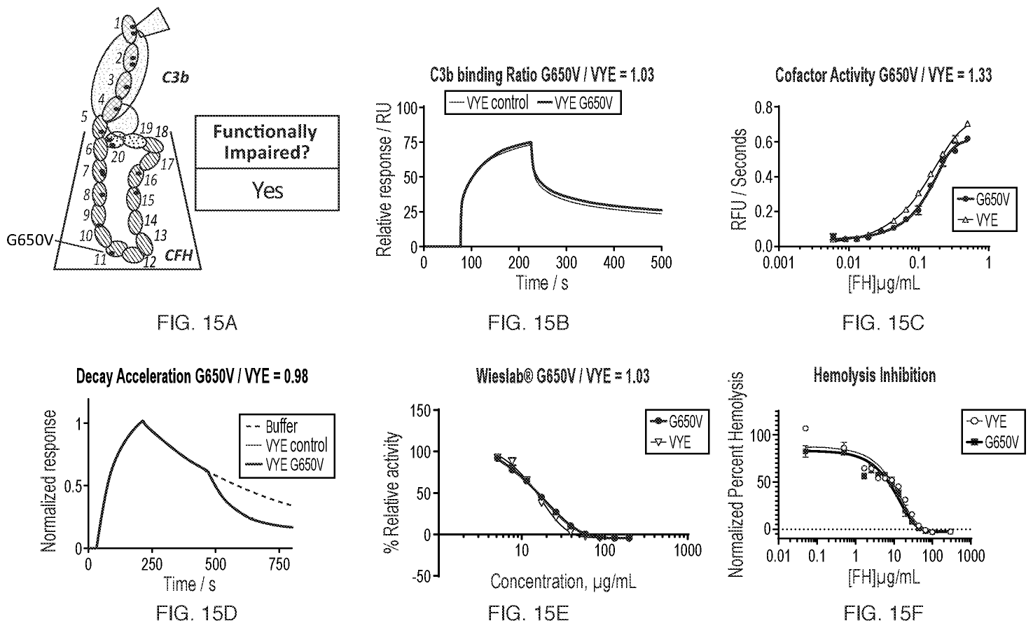

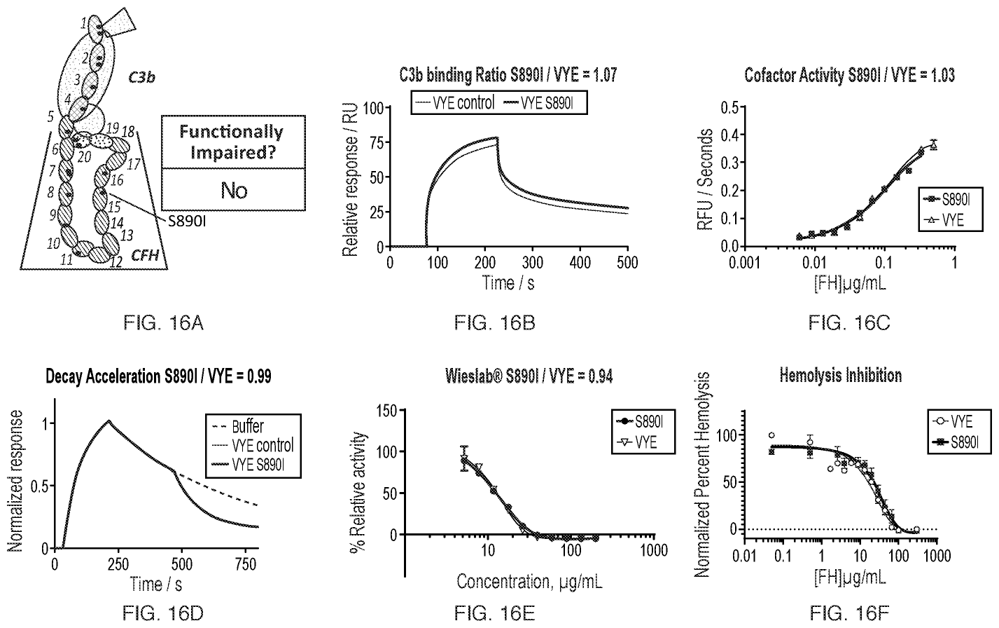

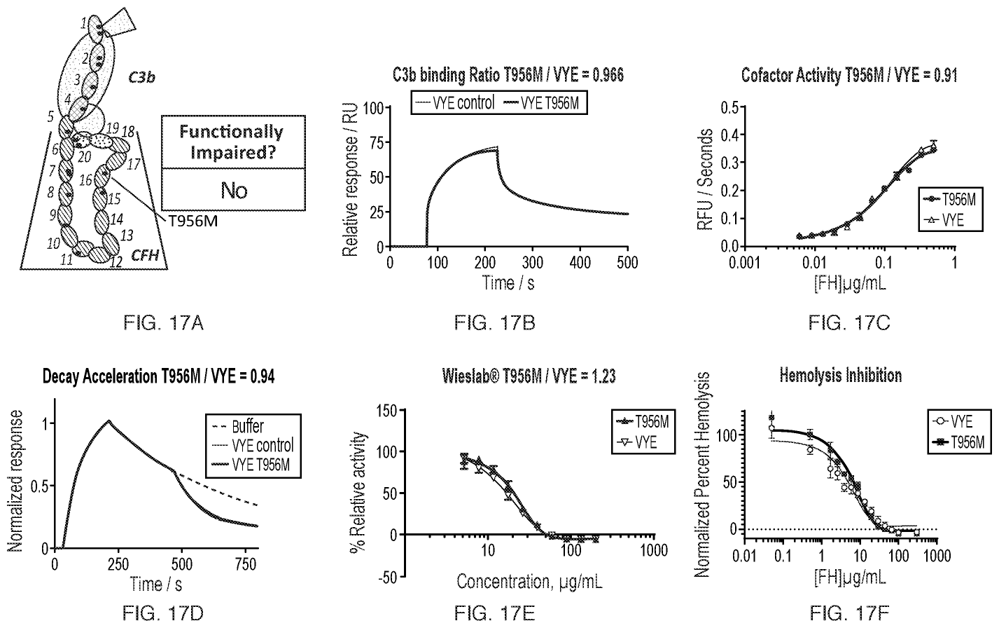

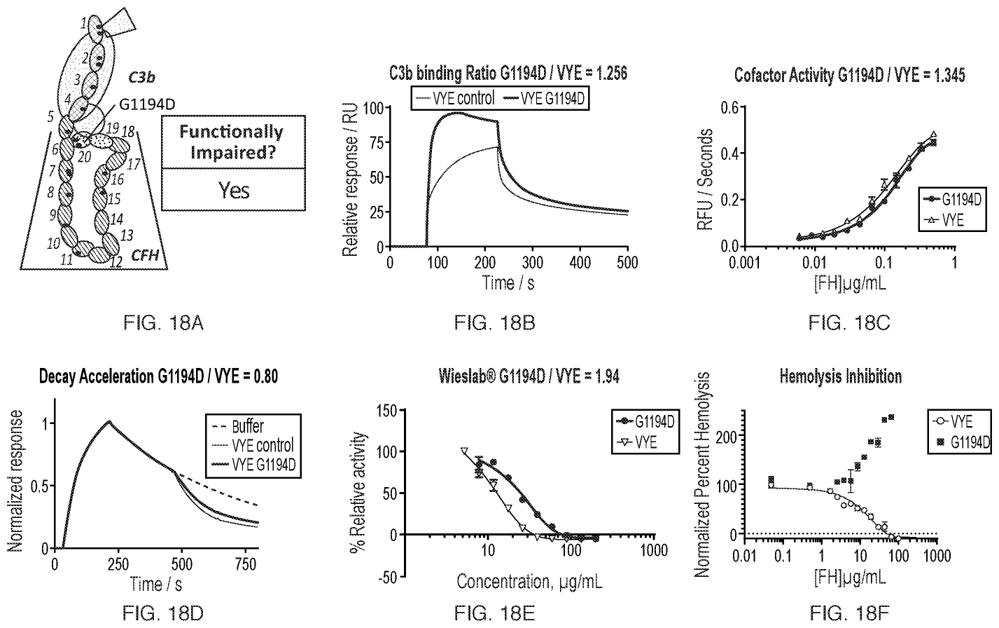

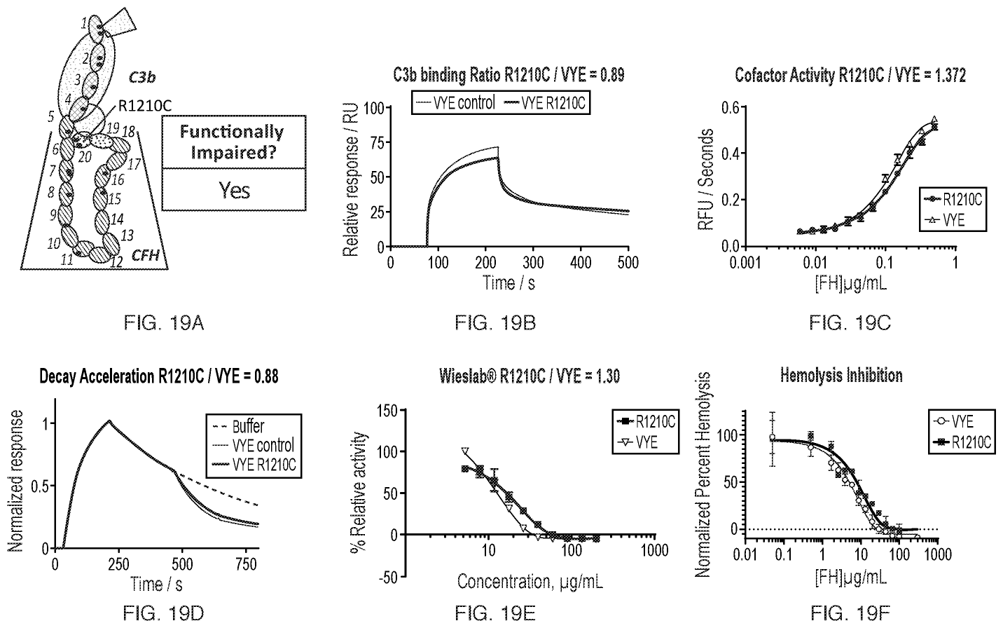

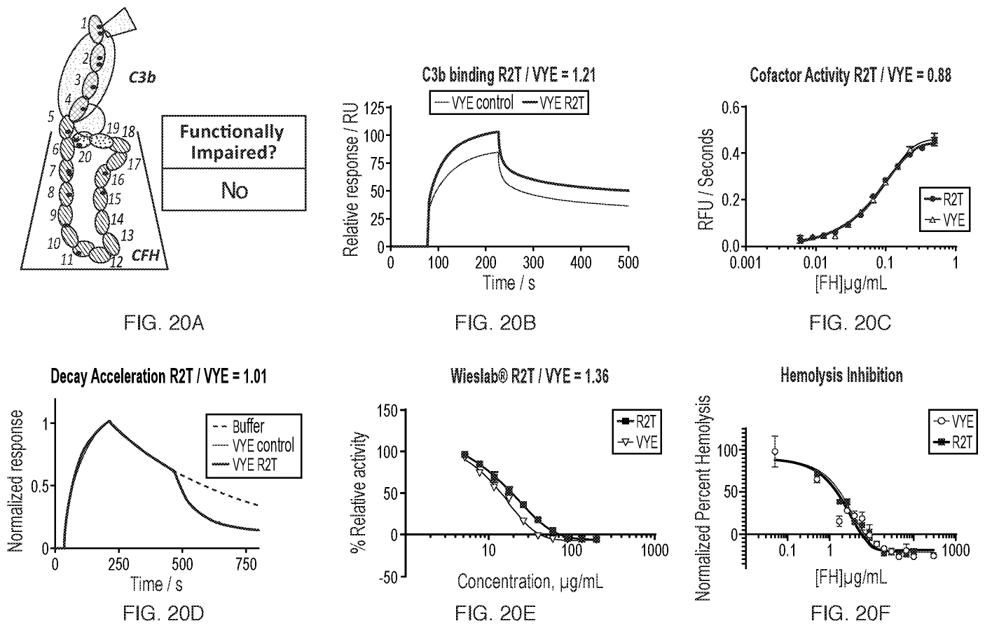

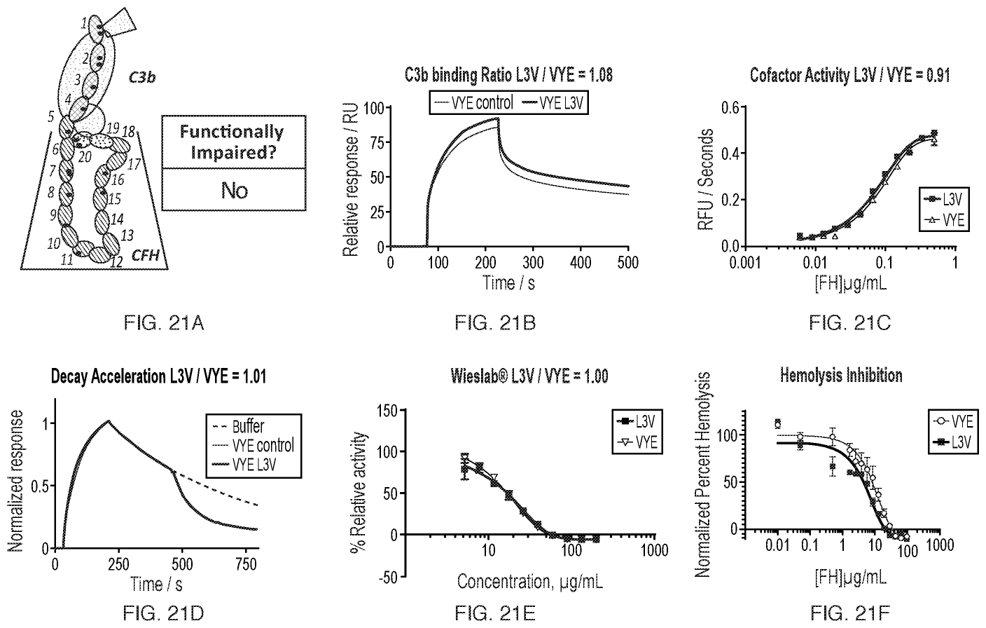

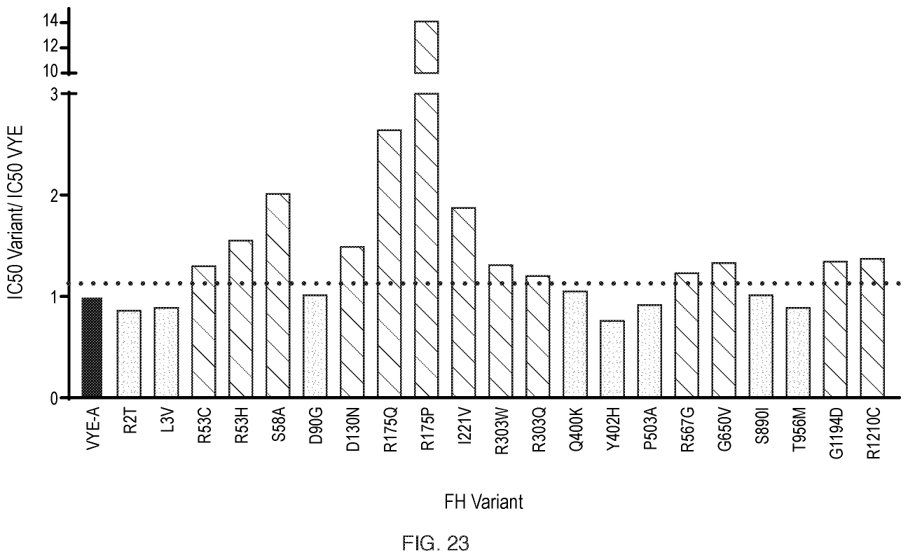

The inverse reaction rates (corresponding to C3b cleavage), at each concentration of CFH were plotted on log scale and fit to calculate the EC₅₀ value. See FIGS. 1C, 2C, 3C, 4C, 5C, 6C, 7C, 8C, 9C, 10C, 11C, 12C, 13C, 14C, 15C, 16C, 17C, 18C, 19C, 20C, 21C. The ratio of the EC₅₀ for each CFH Variant over the EC₅₀ for VYE was calculated. A variant with a ratio>1.2 was considered to be functionally impaired compared to VYE. See FIG. 23.

### Hemolysis Assay

Human CFH-depleted serum (20 μL) was preincubated with a serially diluted set of concentrations of rhCFH variants ranging from 0.5 μg/mL to 300 μg/mL (CFH concentrations were relative to the volume of serum) in gelatin-containing veronal buffer (GVB° with 0.1 mM EDTA) at room temperature for 10 minutes. SEs were washed once in GVB° without EDTA and resuspended to 2.1×10⁸ cells/mL in GVB° with 0.1 mM EDTA. A total of 2.1×10⁷ SE were added to each well followed by Mg-EGTA at a final concentration of 10 mM in a total reaction volume of 200 μL. Wells containing reaction mixes in heat-inactivated, CFH-depleted serum and 1% triton-X in water were included as a negative and positive control for lysis, respectively. Samples were incubated at 37° C. for 60 minutes with shaking, followed by the addition of 150 μL of GVBE to stop further cell lysis. The samples were centrifuged at 1000×g for 5 minutes at 7° C. A volume of 150 μL of precleared supernatant was transferred to a clear bottom 96-well plate and the extent of hemolysis was measured in triplicate samples for each CFH concentration as absorbance at 412 nm (maximum absorbance for hemoglobin), corrected for background absorbance measured at 690 nm. All reagents, instruments and laboratory supplies used in this experiment are listed in Tables 3 and 4.

TABLE 3
Equipment and supplies for hemolysis assay

| Equipment | Manufacturer |
|---|---|
| SpectraMax M5e | Molecular Devices |
| Pipette (2 μL) | VistaLab |
| Pipette (20 μL) | VistaLab |
| Pipette (200 μL) | VistaLab |
| Pipette (1000 μL) | VistaLab |
| Multichannel pipette (20 μL) | VistaLab |
| Multichannel pipette (200 μL) | VistaLab |
| Shaking incubator | New Brunswick Scientific |
| 96 well deep well v-bottom plates | Costar |
| 96 well clear plates | Corning |
| Plate sealers | Thermo |
| Refrigerated centrifuge | Beckman |
| Water bath | Thermo |
| Prism 7 for Windows | GraphPad |

TABLE 4
Reagents for hemolysis assay

| Reagent | Manufacturer | Concentration | Storage |
|---|---|---|---|
| Gelatin-containing | Complement | 0.1% gelatin, 5 mM | 4° C. |
| veronal buffer (GVB^{o}) | Technology | Veronal, 145 mM | |
| | | NaCl, 0.025% NaN3, | |
| | | pH 7.3 | |
| Gelatin-containing | Complement | 0.1% gelatin, 5 mM | 4° C. |
| veronal buffer with 10 | Technology | Veronal, 145 mM | |
| mM EDTA (GVBE) | | NaCI, 0.025% NaN3, | |
| | | 10 mM EDTA | |
| | | pH 7.3 | |
| Triton-X | Biovision | 10% in water | 4° C. |
| Mg•EGTA | Complement | 0.1M Mg•EGTA, pH | 4° C. |
| | Technology | 7.3 | |
| CFH-depleted human | Complement | n/a | −80° C. |
| serum | Technology | | |
| Sheep erythrocytes | Complement | 5 × 10⁸ cells/mL | 4° C. |
| | Technology | | |
| rhCFH (155 kDa) | Thermo | Various concentrations; | 4° C. |
| | | dilute with 1X TBS | |
| | | between 0.0005 mg/mL | |
| | | to 0.3 mg/mL | |

Abbreviations:
EDTA, Ethylenediaminetetraacetic acid;
EGTA, ethylene glycol-bis(β-aminoethyl ether)-N,N,N′,N′-tetraacetic acid;
kDa, kilodaltons

Corrected absorbance measurements were expressed as percentage of the 100% lysis control (CFH-depleted serum alone). The baseline hemolysis level was set using SEs incubated with heat-inactivated, CFH-depleted serum. The formula used to normalize each measurement to percent hemolysis is shown in Formula 1.

$$Formula 1 : Normalized percent hemolysis formula \\ normalized percent hemolysis = \frac{Xi - min ( X )}{max ( X ) - min ( X )} \times 100 ,$$

$$Xi is the individual measured value for each sample , max ( X ) is average of the values from triplicate samples incubated with CFH - depleted serum and min ( X ) is the average of the values from triplicate samples incubated with heat - inavtivated CFH - delpleted serum$$

For each concentration of CFH variant in pg/mL (on a log scale), the averaged normalized percent hemolysis values plus or minus the standard deviation were plotted. The data were fit with a 4-point sigmoidal curve from which the EC₅₀ value was interpolated. Given the inherent variability of this assay, a numerical basis for defining functional impairment was not possible. Variants with a functional impairment were defined for those with an unequivocal difference in potency in the assay compared to VYE or, in cases where the effects were subtle, a trend towards less activity than VYE in at least 2 of 3 independent runs of the assay was used as a basis of defining functional impairment.

The extent of hemolysis was measured in CFH-depleted serum as well as serum enriched with each CFH variant. The average normalized percent hemolysis values plus or minus the standard deviation is plotted. The data were fit to interpolate the EC50 value. See FIGS. 1F, 2F, 3F, 4F, 5F, 6F, 7F, 8F, 9F, 10F, 11F, 12F, 13F, 14F, 15F, 16F, 17F, 18F, 19F, 20F, 21F.

### C3b Binding Affinity by SPR

SPR experiments were performed at 25° C. on a Biacore T200 instrument (GE Healthcare). A total of 300 RUs of C3b (supplied by Complement Technology, Inc., lot number 26a) were attached using standard amine coupling to one flow cell of a Biacore CM1 chip (Lot number: 10260563) (GE Healthcare). A second flow cell of the CM1 chip was used for background subtraction.

A 1 μM solution of each CFH variant, in 10 mM 4-(2-hydroxyethyl)-1-piperazineethanesulfonic acid (HEPES) buffer, 150 nM NaCl, 0.05% (v/v) surfactant P20, (pH 7.4) (GE Healthcare), was injected over amine-coupled C3b for 150 seconds at 50 μL/minute followed by a dissociation time of 600 seconds. The sensor chip surface was regenerated between individual injections by three 30-second 50-μL/minute injections of 1 M NaCl, followed by a surface stabilization period of 150 seconds after the third NaCl injection. The baseline drift was corrected by subtracting the signal obtained from an injection of 0 μM CFH. Data were analyzed using the Biacore Evaluation software and a 1:1 steady-state binding model. For comparison, an identically prepared solution of VYE was flowed over the same flow cell. The corrected responses were then plotted against time, and the resultant plots overlaid at the time point of VYE/variant injection. The ratio C3b binding for each variant was compared to VYE and variants with ratios below 0.90 in duplicate independent experiments were considered to be functionally impaired for this activity. All reagents, instruments, and laboratory supplies used in this experiment are listed in Tables 5 and 6.

TABLE 5
Equipment and supplies for SPR experiments

| Equipment | Manufacturer |
|---|---|
| Biacore T200 | GE Healthcare |
| Pipette (10 μl) ErgoOne ® | STARLAB |
| Pipette (100 μl) ErgoOne ® | STARLAB |
| Pipette (200 μl) ErgoOne ® | STARLAB |
| Pipette (1000 μl) ErgoOne ® | STARLAB |
| Biacore C1 chip | GE Healthcare |
| Slide-A-Lyzer ® Mini Dialysis | Thermo Scientific |
| Units | |
| Tube-O-Dialyzer ™ Medi | G-Biosciences |
| Biacore Evaluation software | GE Healthcare |
| Prism 7 for Windows | GraphPad |

TABLE 6
Reagents for SPR experiments

| Reagent | Manufacturer | Concentration | Storage |
|---|---|---|---|
| 10 x HBS-P+ | GE Healthcare | Diluted to 1 x, i.e., 10 | ambient |
| | | mM HEPES buffer, 150 | |
| | | nM NaCI, 0.05% (v/v) | |
| | | surfactant P20, (pH 7.4) | |
| Complement | Complement | 1.03 mg/mL | −80° C. |
| component 3b | Technology, | | |
| | Inc. | | |
| rhCFH variants | Thermo | Various concentrations; | 4° C. |
| (155 kDa) | | diluted with 1 x HBS- | |
| | | P+, to 1 μM | |

Abbreviations:
HBS-P+, HEPES-buffered saline with P20;
kDa, kilodaltons

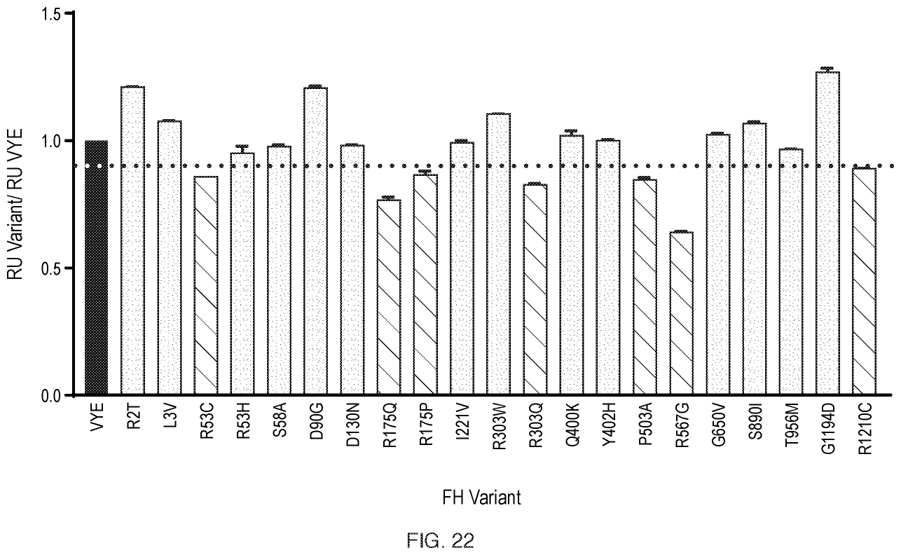

The C3b binding activity for each CFH variant was compared to the VYE control to determine the C3b binding ratio. See FIGS. 1B, 2B, 3B, 4B, 5B, 6B, 7B, 8B, 9B, 10B, 11B, 12B, 13B, 14B, 15B, 16B, 17B, 18B, 19B, 20B, 21B. Variants with ratios below 0.90 were considered functionally impaired for C3b binding activity. See FIG. 22.

### Decay Acceleration Activity (DAA) Assay

The SPR experiments were performed at 25° C. on a Biacore T200 instrument (GE Healthcare) using 10 mM 4-(2-hydroxyethyl)-1-piperazineethanesulfonic acid (HEPES), 150 mM NaCl, 0.05% (v/v) surfactant P20, 1 mM MgCl2 (pH 7.4) (GE Healthcare). A total of 2845 response units (RUs) of C3b (lot number 26a) was immobilized to a flow cell of a Biacore CM5-sensor chip (Lot number: 10260535) using standard amine coupling. A reference flow cell was used for background subtraction. C3bBb was assembled on the CM5 chip bearing immobilized C3b molecules, by performing a 180 second 10-μL/minute injection of a solution containing 500 nM CFB (lot number 17b) and 50 nM CFD (lot number 38). The decay of C3bBb was monitored over an initial 240 second dissociationphase in the absence ofany CFH, allowing observation of intrinsic convertase decay (i.e., a decline in RUs attributable to loss of Bb). Subsequently, a 20 nM solution of either VYE or a CFH variant were injected at 10 μL/minute for 180 seconds, allowing observation of accelerated dissociation. All reagents, instruments, and laboratory supplies used in this experiment are listed in Table 7 and 8.

TABLE 7
Equipment and supplies for DAA assay

| Equipment | Manufacturer |
|---|---|
| Biacore T200 | GE Healthcare |
| Pipette (10 μL) | STARLAB |
| Pipette (100 μL) | STARLAB |
| Pipette (200 μL) | STARLAB |
| Pipette (1000 μL) | STARLAB |
| Biacore CM5 chip | GE Healthcare |
| Slide-A-Lyzer ® Mini Dialysis | Thermo Scientific |
| Units | |
| Tube-O-Dialyzer ™ Medi | G-Biosciences |
| Biacore Evaluation software | GE Healthcare |
| Prism 7 for Windows | GraphPad |

TABLE 8
Reagents for DAA assay

| Reagent | Manufacturer | Concentration | Storage |
|---|---|---|---|
| 10 x HBS-P+ | GE Healthcare | Diluted to 1 x, i.e., 10 mM | ambient |
| | | HEPES buffer, 150 nM NaCl, | |
| | | 0.05% (v/v) surfactant P20, | |
| | | and supplemented with 1 mM | |
| | | MgCl₂ (pH 7.4) | |
| Complement | Complement | 1.0 mg/mL | −80° C. |
| factor B | Technology, Inc. | | |
| Complement | Complement | 0.1 mg/mL | −80° C. |
| factor D | Technology, Inc. | | |
| Complement | Complement | 1.03 mg/mL | −80° C. |
| component | Technology, Inc. | | |
| 3b | | | |
| rhCFH | Thermo | Various concentrations; | 4° C. |
| variants | | diluted with 1 x HBS-P+, 1 | |
| | | mM MgCl₂, to 20 nM | |

The SPR response arising from the binding of each CFH variant to immobilized C3b, in the absence of the convertase, was subtracted from the corresponding convertase decay response. Data were processed using the Biacore Evaluation software. Experimental data were then normalized to compensate for the small drift in signal (assumed to arise from the gradual leaching of C3b from the surface over multiple measurements). Normalization of data was achieved by comparing responses at 220 seconds (i.e., at the time when the injection of CFB and CFD ceased) and adjusting these to be 1.0. The normalized responses were then plotted against time, and the resultant plots overlaid at the time point of VYE/variant injection.

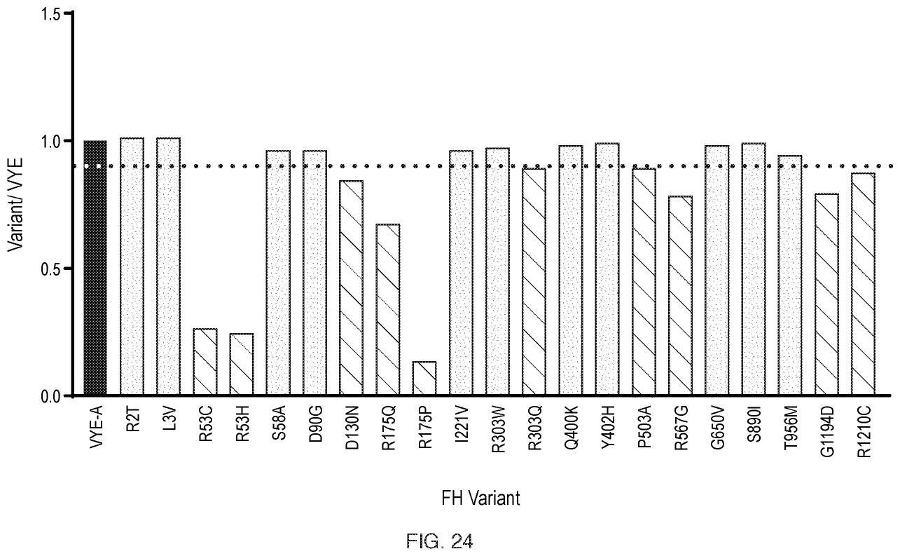

Decay of the surface plasmon resonance (SPR) response of C3 convertase (C3bBb) dissociation of Bb from C3b. Addition of VYE accelerates the dissociation of the C3 convertase and the decay acceleration of each CFH variant was compared to that of VYE. Normalized responses are plotted against time. See FIGS. 1D, 2D, 3D, 4D, 5D, 6D, 7D, 8D, 9D, 10D, 11D, 12D, 13D, 14D, 15D, 16D, 17D, 18D, 19D, 20D, 21D. The ratios of DAA for each variant were compared to VYE and variants with ratios below 0.85 were considered to be functionally impaired for this activity. See FIG. 24.

### Weislab Assay

The Wieslab kit (Cat #COMPLAP330) was used as directed with the following modifications. Instead of NHS, human serum depleted of CFH (Complement Technology; A337; Lot #11) that was supplemented with either VYE or a CFH variant in a dose response from 5.2 to 200 ug/ml was utilized with the kit components. All other kit reagents were used as directed. The data for each variant were normalized to the activity of CFH depleted serum alone and the relative activity was plotted against CFH concentration in ug/ml. The ratio of each variant's interpolated IC50 over that for VYE was calculated and values greater than 1.6 were used to determine functional impairment.

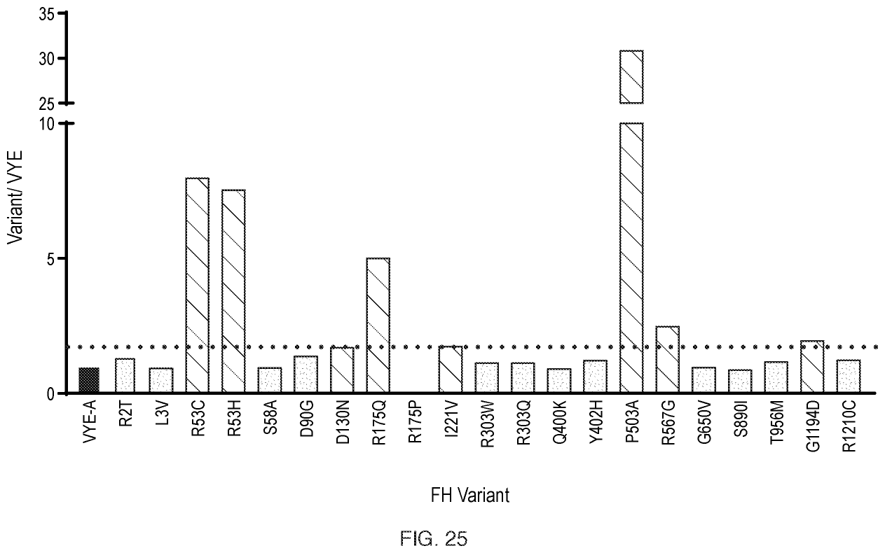

The Wieslab® kit (Svar Life Science AB) was used to determine the percent activity of serum enriched with each CFH variant normalized to the activity of CFH depleted serum alone. The relative activity was plotted against CFH concentration in g/ml. The ratio of each variant's interpolated IC50 over that for VYE was calculated and CFH variants having values greater than 1.6 were considered functionally impaired. See FIGS. 1E, 2E, 3E, 4E, 5E, 6E, 7E, 8E, 9E, 10E, 11E, 12E, 13E, 14E, 15E, 16E, 17E, 18E, 19E, 20E, 21E. See also FIG. 25.

### Cellular Expression Assay

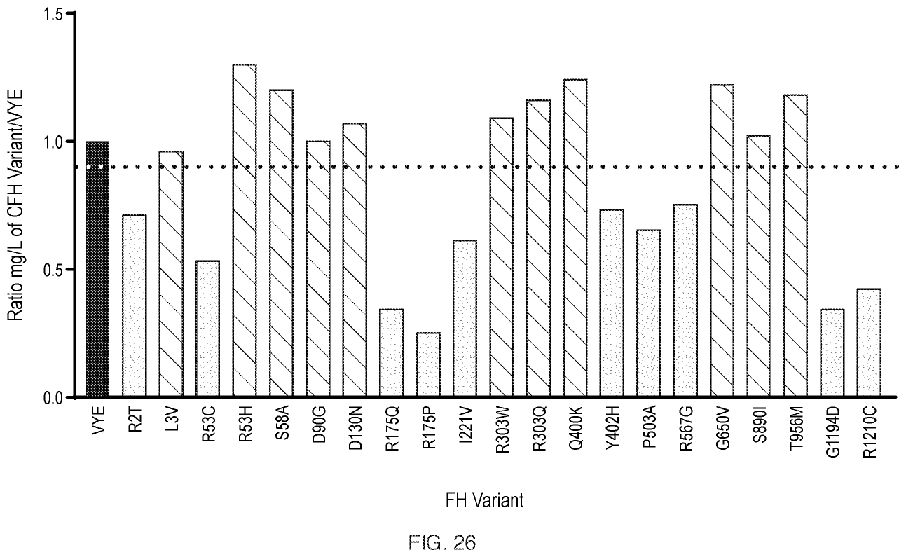

DNA plasmids encoding each of the variants were prepared and transfected transiently into cells. Cells were cultured for five days and the supernatants collected and cells removed. The CFH variant in each of the supernatants was then purified using a proprietary CFH affinity resin. Upon elution and pooling of fractions containing CFH the protein concentration was measured and the total protein recovered was calculated. For each variant the amount of protein produced per unit volume was compared to wild type CFH and the ratio calculated. The ratio of cellular expression of each CFH variant compared to expression of the CFH control was plotted. Functionally deficient CFH variants were determined to have a ratio<0.9. See FIG. 26.

### SUMMARY

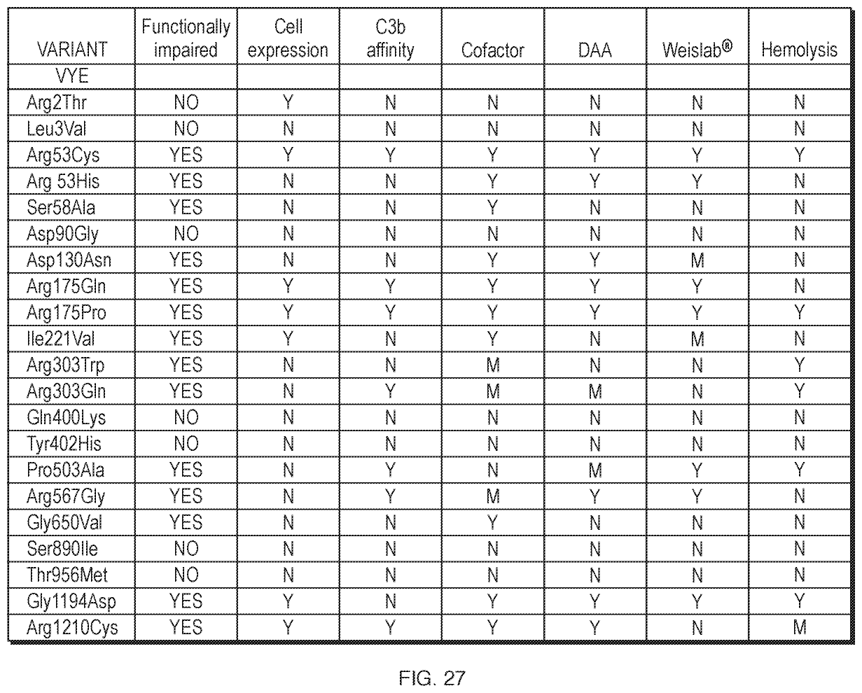

FIG. 27 shows summary functional assay data for each CFH variant tested as compared to VYE control CFH.

### Example 2: Construction of AAV Vectors

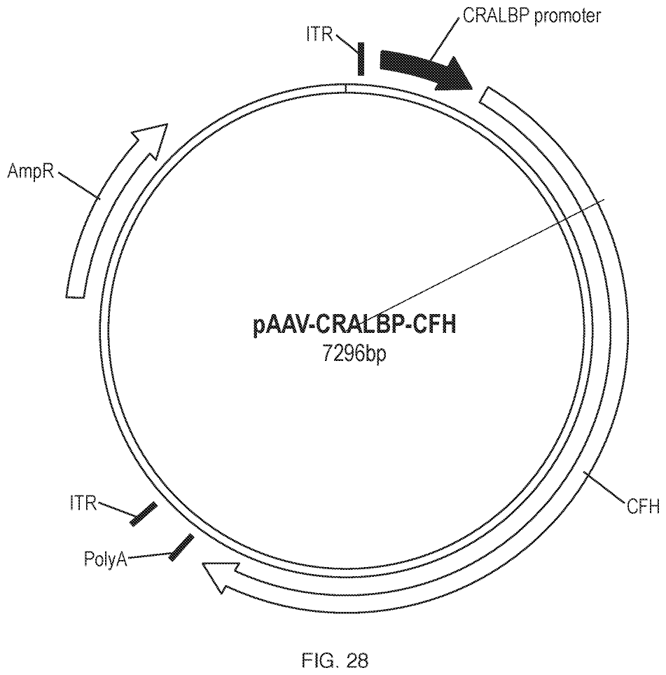

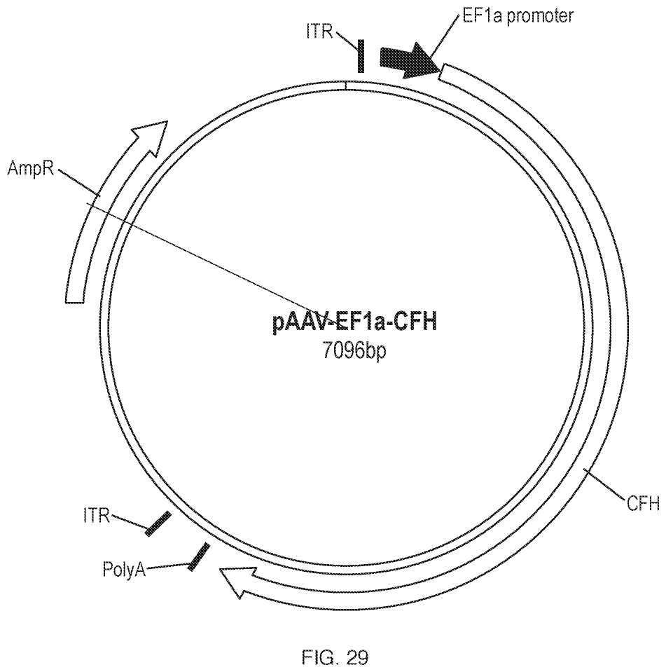

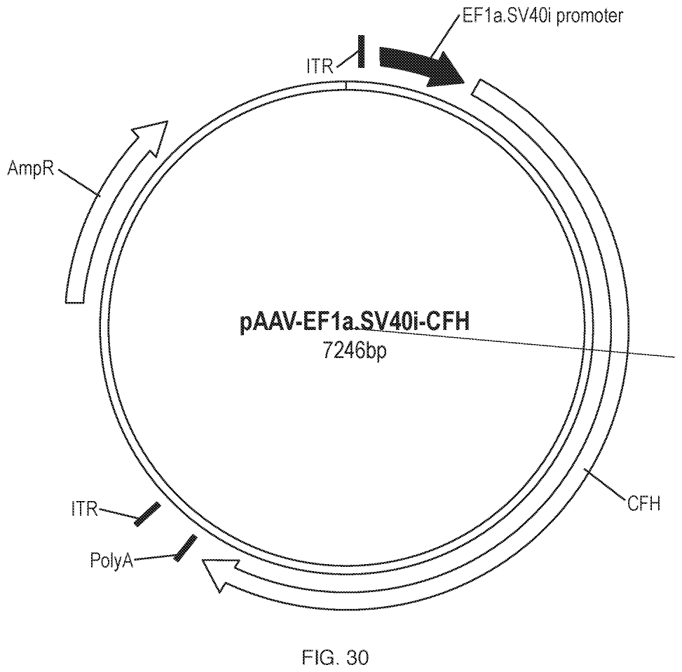

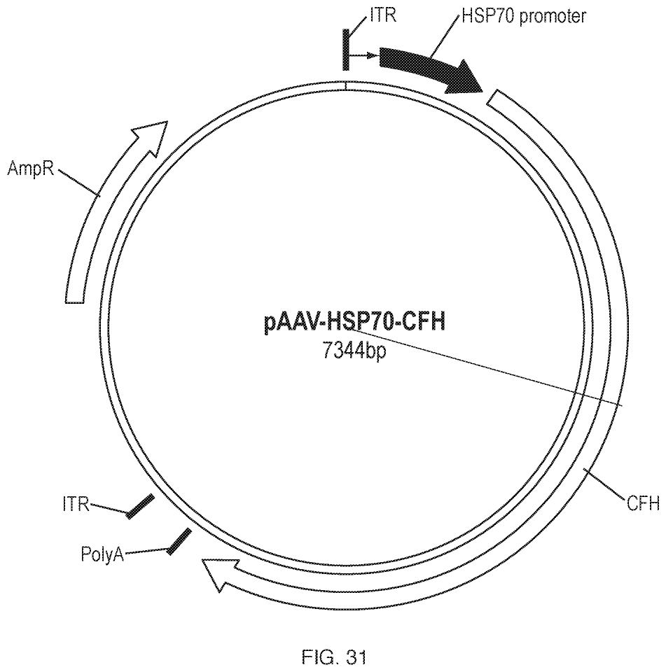

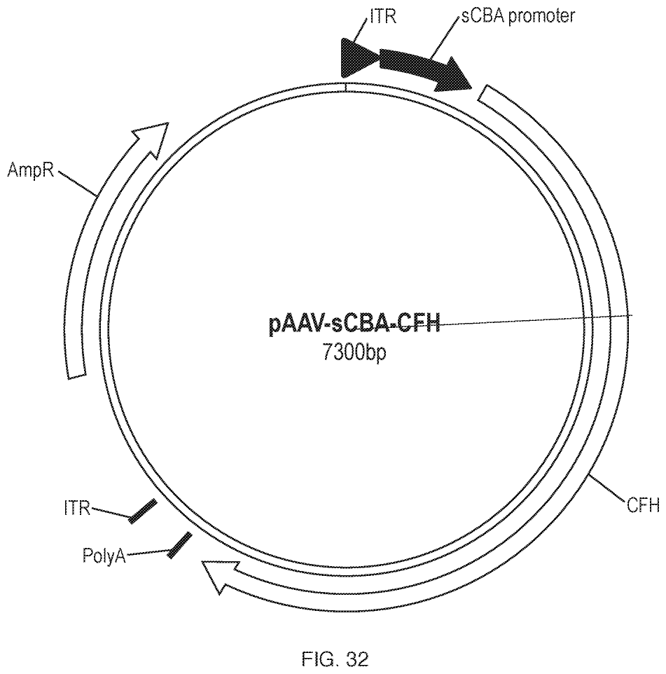

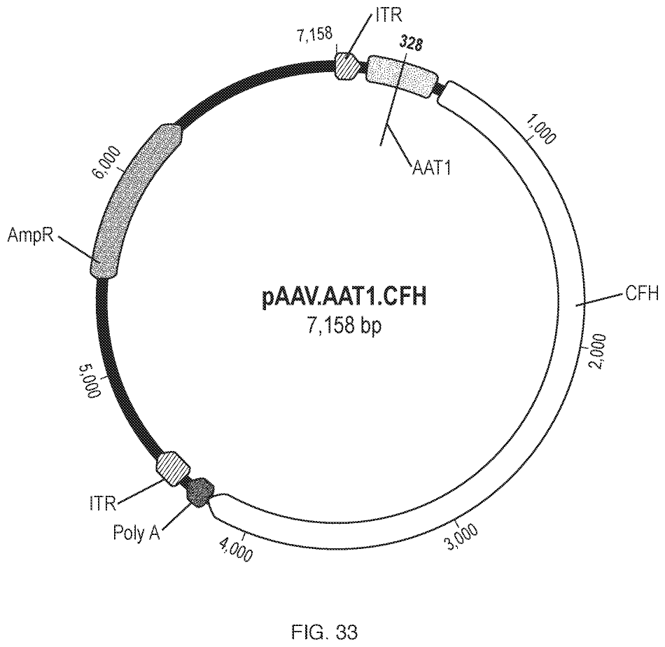

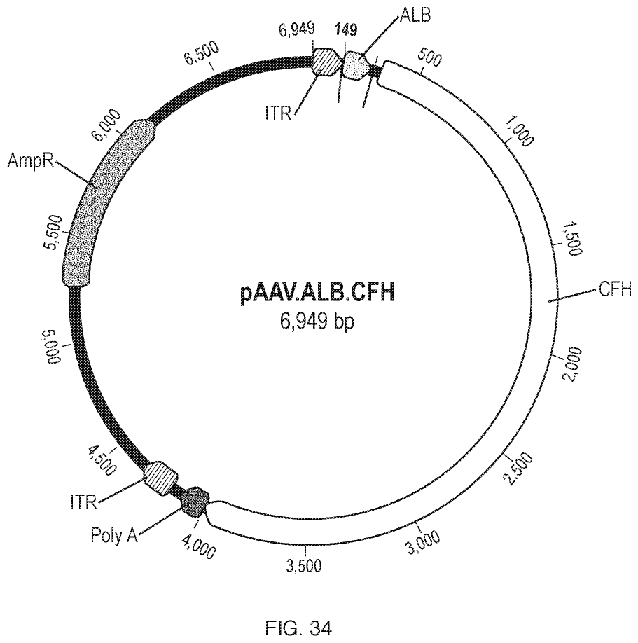

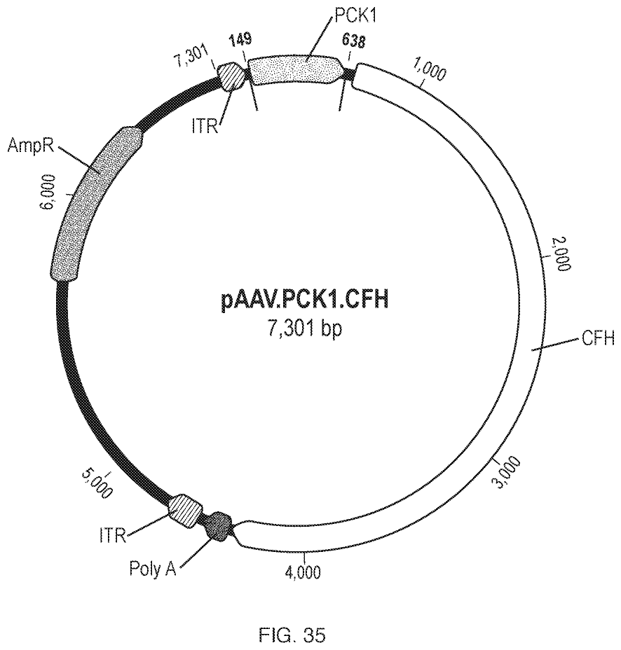

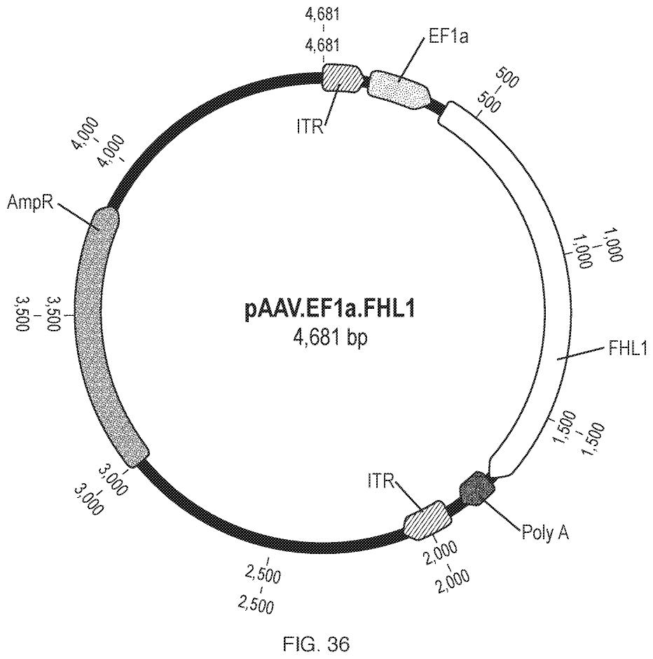

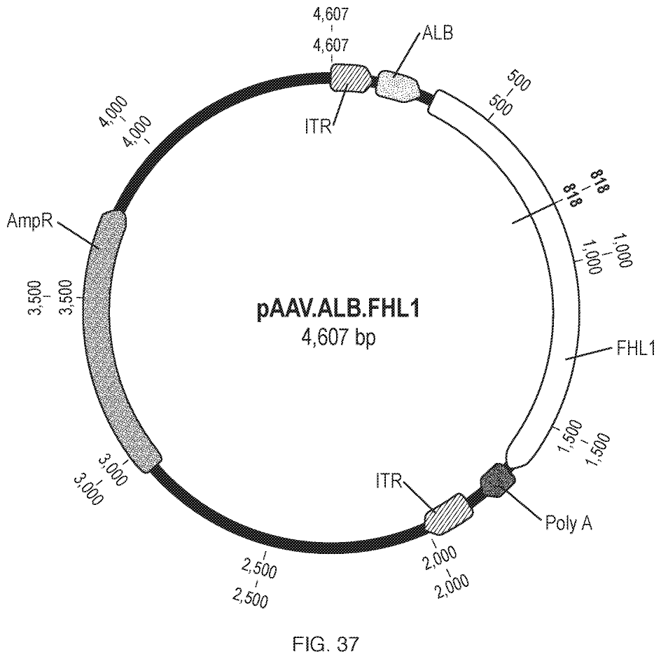

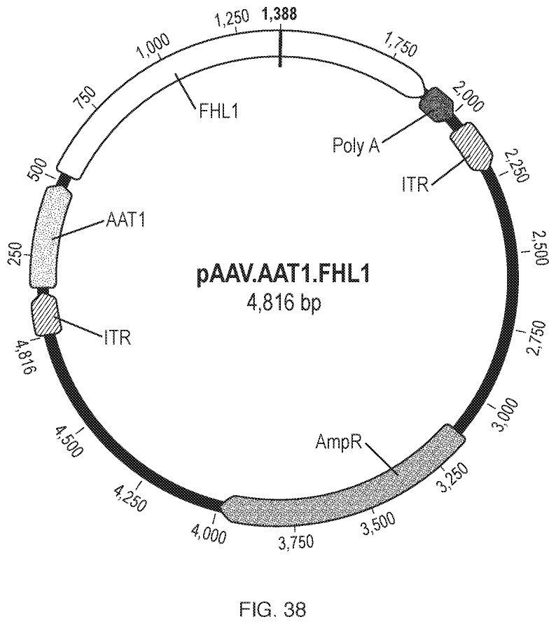

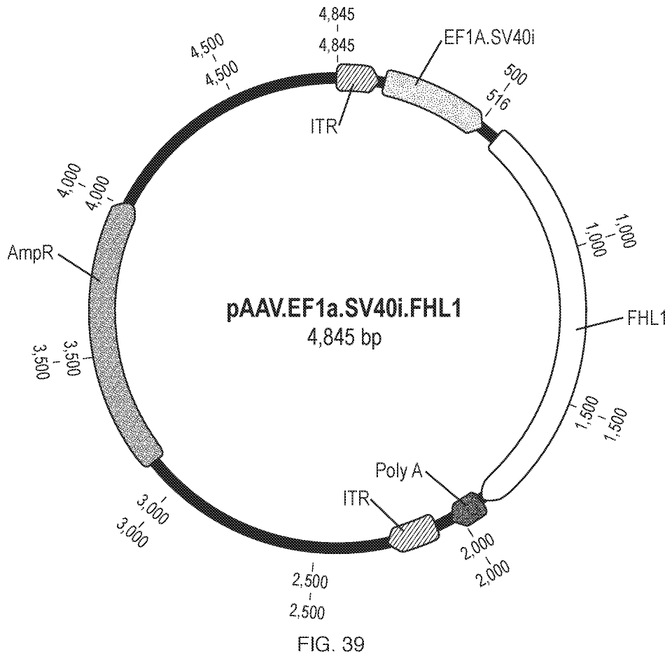

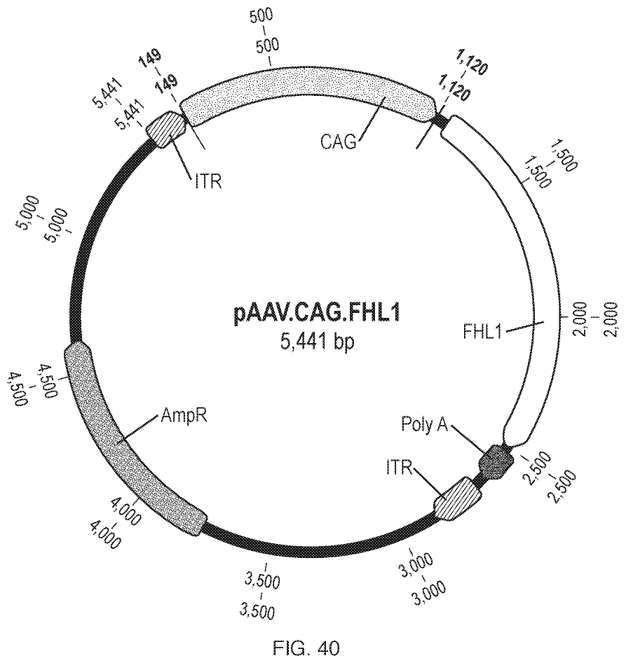

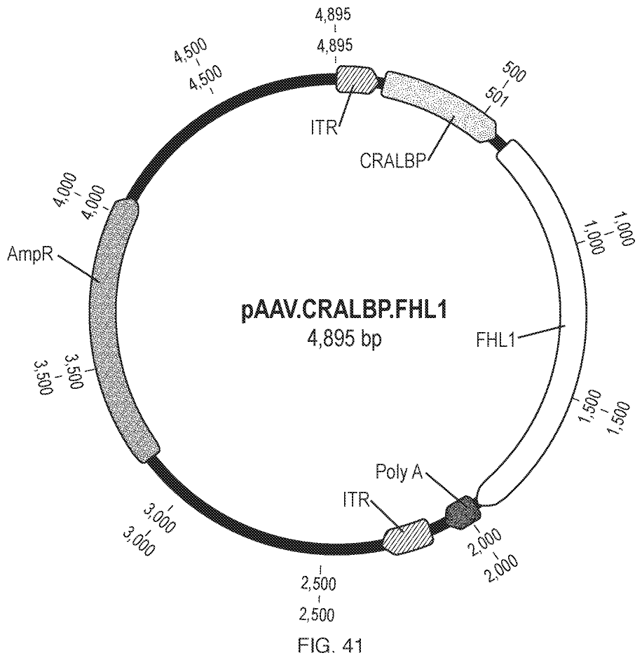

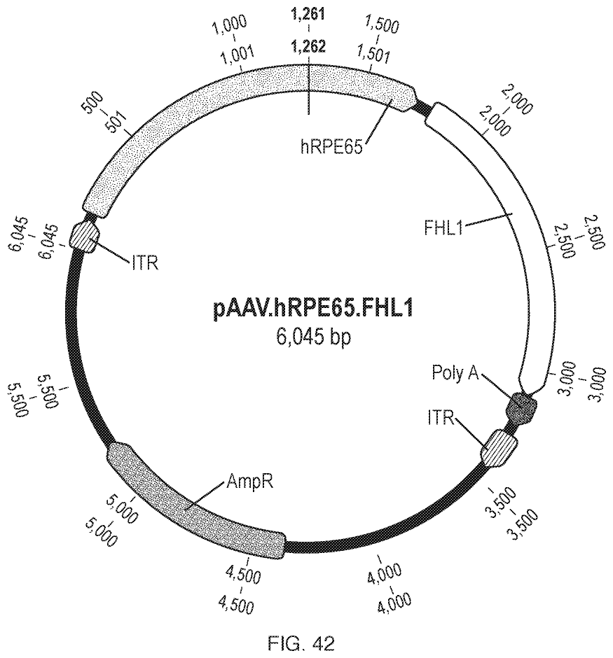

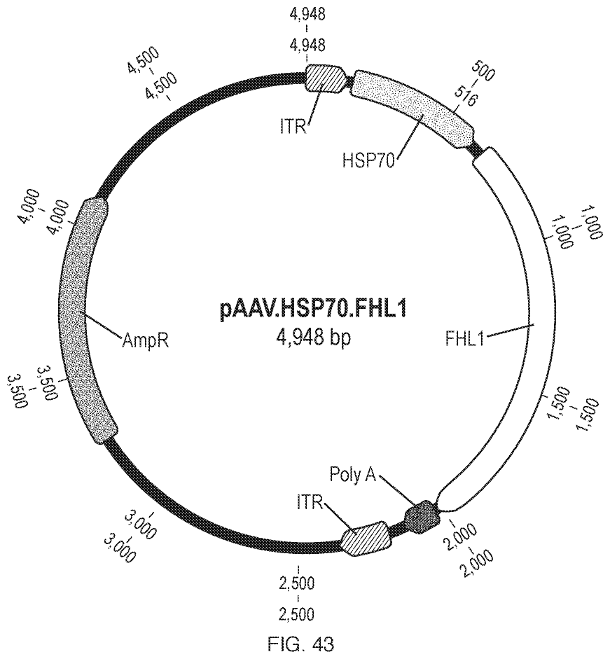

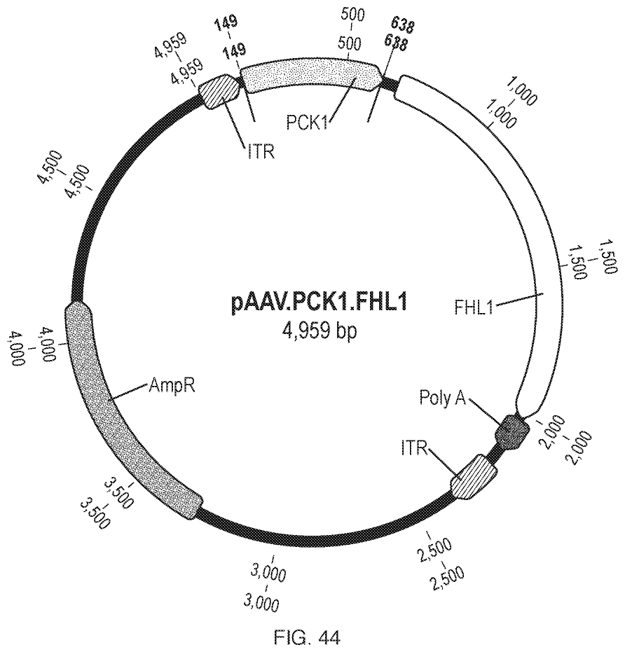

AAV2 vectors were designed comprising either codon-optimized or non-codon-optimized CFH and/or CFHL sequences in combination with a variety of different promoters and, in some cases, SV40 introns. FIGS. 28-44 show vector maps of the different vectors generated. Table 9 below outlines the gene included in the cassette, the promoter included, the Figure laying out the construct map, and the sequence associated with the vector.

TABLE 9
Exemplary AAV Vectors

| | | | | Construct |
|---|---|---|---|---|
| Construct Name | Transgene | Promoter | FIG. | Sequence |
| pAAV-CRALBP- | CFH | CRALBP | 28 | 7 |
| CFH | | | | |
| pAAV-EF1a-CFH | CFH | EF1a | 29 | 9 |
| pAAV-EF1a- | CFH | EF1a | 30 | 11 |
| SV40i-CFH | | | | |
| pAAV-HSP70- | CFH | HSP70 | 31 | 13 |
| CFH | | | | |
| pAAV-sCBA-CFH | CFH | CBA | 32 | 15 |
| pAAV-AAT1-CFH | CFH | AATI | 33 | 17 |
| pAAV-ALB-CFH | CFH | ALB | 34 | 19 |
| pAAV-PCK1-CFH | CFH | PCK1 | 35 | 21 |
| pAAV-EF1a- | CFHL | EF1a | 36 | 22 |
| CFHL | | | | |
| pAAV-ALB-CFHL | CFHL | ALB | 37 | 23 |
| pAAV-AAT1- | CFHL | AAT1 | 38 | 24 |
| CFHL | | | | |
| pAAV-EF1a- | CFHL | EF1a | 39 | 25 |
| SV40i-CFHL | | | | |
| pAAV-CAG- | CFHL | CAG | 40 | 26 |
| CFHL | | | | |
| pAAV-CRALBP- | CFHL | CRALBP | 41 | 27 |
| CFHL | | | | |
| pAAV-hRPE65- | CFHL | hRPE65 | 42 | 28 |
| CFHL | | | | |
| pAAV-HSP70- | CFHL | HSP70 | 43 | 29 |
| CFHL | | | | |
| pAAV-PCK1- | CFHL | PCK1 | 44 | 30 |
| CFHL | | | | |

### Ability of AAV.CFH and AAV.FHL1 Vectors to Transduce Cells and Regulate Complement Activity

The CFH vectors indicated above each are first tested in vitro in ARPE19 cells via transfection and evaluated for expression of the human CFH and FHL1 protein in both cell pellets and in the supernatant. Techniques like Western blot are used for protein detection and quantification. Quantitative Real time PCR are used for determining mRNA expression levels. Regulation of complement activity is tested in a cell culture model of blue light irradiation of A2E-laden retinal pigment epithelial cells as described in van der Burght et al, Acta Ophthalmol, 2013. Briefly, ARPE-19 cell line is grown to confluence and cultured in standard media plus or minus 10 uM A2E for 4 weeks. RPE are irradiated with blue light. Media is replaced with PBS plus calcium, magnesium and 5.5 mM glucose and cells are irradiated with blue light (430+/−30 nm) for 0, 5 or 10 minutes. RPE cells are incubated with appropriately-complement depleted human serum+/− and transfected with the AAV.CFH and AAV.FHL1 vectors. Immunoreactivity of RPE with cell surface markers, CD46, CD55 and CD59 and C3 and MAC deposition will be assessed by fluorescent microscopy or western blot. Levels of iC3b will be measured by Western Blot.

After evaluation in ARPE19 cells, the AAV.CFH and AAV.FHL1 vectors are tested in mouse models of light-induced retinal degeneration and laser induced choroidal neovascularization via intravitreal injections. Amount of protein produced and its biodistribution in the retina is tested via Western blot and immunohistochemistry. Rescue of photoreceptor thinning and RPE cell death is assessed via optical coherence tomography, fundus photography and histological analyses. Immunoreactivity of RPE with cell surface markers, CD46, CD55 and CD59 and C3 and MAC deposition is assessed by fluorescent microscopy or western blot. Levels of iC3b (cleavage product of C3) is measured by Western Blot.

Appropriate dose for non-human primates is determined based on mouse studies. Non-human primate studies is conducted in cynomolgus monkeys via intravitreal injections. Therapeutic benefits is evaluated based on levels of CFH and FHL1 proteins produced and secreted by the RPE. Amount of secreted CFH and FHL1 protein is measured in the retina and the choroid compared to uninjected or sham injected cohorts. Increased levels of CFH and FHL1 in the retina and choroid is expected to provide therapeutic benefits in the AMD population with rare mutations that lead to the loss or decreased amount of these protein.

### Example 3: Treatment of Patients with AMD with AAV Vectors

This study evaluates the efficacy of the vectors of Example 2 for treating patients with AMD having any one or more of the mutations referenced in Example 1. Patients with AMD are treated with any of the CFH AAV2 vectors, or a control. The vectors are administered at varying doses between 2.5×10⁸ vg to 1.4×10¹¹ vg/per eye in about 100 μl. The vectors are administered by intravitreal injection in a solution of PBS with additional NaCl and pluronic. Patients are monitored for improvements in AMD symptoms.

### Numbered Embodiments

Embodiments disclosed herein include embodiments P1 to P126 as provided in the numbered embodiments of the disclosure.

Embodiment P1: A method of treating a subject having a disease or disorder associated with undesired activity of the alternative complement pathway, comprising the step of administering to the subject an adeno-associated viral (AAV) vector encoding a Complement Factor H (CFH) polypeptide or biologically active fragment or variant thereof, and wherein the subject has one or more CFH gene mutations.

Embodiment P2: A method of treating a subject having age-related macular degeneration (AMD), comprising the step of administering to the subject an adeno-associated viral (AAV) vector encoding a Complement Factor H (CFH) polypeptide or biologically active fragment or variant thereof, and wherein the subject has one or more CFH gene mutations.

Embodiment P3: The method of embodiment P1 or P2, wherein the nucleotide sequence is at least 80% identical to the nucleotide sequence of SEQ ID NO: 1-3 or 5, or codon-optimized variant and/or a fragment thereof.

Embodiment P4: The method of embodiment P1 or P2, wherein the nucleotide sequence is at least 90% identical to the nucleotide sequence of SEQ ID NO: 1-3 or 5, or codon-optimized variant and/or a fragment thereof.

Embodiment P5: The method of embodiment P1 or P2, wherein the nucleotide sequence is the sequence of SEQ ID NO: 1-3 or 5, or codon-optimized variant and/or a fragment thereof.

Embodiment P6: The method of any one of embodiments P1-P5, wherein the vector encodes a CFH polypeptide or biologically active fragment or variant thereof comprising at least four CCP domains.

Embodiment P7: The method of any one of embodiments P1-P5, wherein the vector encodes a CFH polypeptide or biologically active fragment or variant thereof comprising at least five CCP domains.

Embodiment P8: The method of any one of embodiments P1-P5, wherein the vector encodes a CFH polypeptide or biologically active fragment or variant thereof comprising at least six CCP domains.

Embodiment P9: The method of any one of embodiments P1-P5, wherein the vector encodes a CFH polypeptide or biologically active fragment or variant thereof comprising at least seven CCP domains.

Embodiment P10: The method of any one of embodiments P1-P5, wherein the vector encodes a CFH polypeptide or biologically active fragment or variant thereof comprising at least three CCP domains.

Embodiment P11: The method of any one of embodiments P1-P5, wherein the vector encodes a CFH polypeptide or biologically active fragment or variant thereof comprising the H402 polymorphism.

Embodiment P12: The method of any one of embodiments P1-P5, wherein the vector encodes a CFH polypeptide or biologically active fragment or variant thereof comprising the V62 polymorphism.

Embodiment P13: The method of any one of embodiments P1-P12, wherein the CFH polypeptide or biologically active fragment or variant thereof comprises the amino acid sequence of SEQ ID NO: 4.

Embodiment P14: The method of embodiment P13, wherein the amino acid sequence of SEQ ID NO: 4 is the C-terminal sequence of the CFH protein.

Embodiment P15: The method of any one of embodiments P1-P14, wherein the CFH polypeptide or biologically active fragment or variant thereof is capable of diffusing across the Bruch's membrane.

Embodiment P16: The method of any one of embodiments P1-P15, wherein the CFH polypeptide or biologically active fragment or variant thereof is capable of binding C3b.

Embodiment P17: The method of any one of embodiments P1-P16, wherein the CFH polypeptide or biologically active fragment or variant thereof is capable of facilitating the breakdown of C3b.

Embodiment P18: The method of any one of embodiments P1-P17, wherein the vector comprises a promoter that is less than 1000 nucleotides in length.

Embodiment P19: The method of any one of embodiments P1-P17, wherein the vector comprises a promoter that is less than 500 nucleotides in length.

Embodiment P20: The method of any one of embodiments P1-P17, wherein the vector comprises a promoter that is less than 400 nucleotides in length.

Embodiment P21: The method of any one of embodiments P1-P17, wherein the promoter comprises a promoter having the nucleotide sequence of SEQ ID NO: 6, or a fragment thereof.

Embodiment P22: The method of any one of embodiments P1-P17, wherein the promoter comprises a promoter having the nucleotide sequence of SEQ ID NO: 8, or a fragment thereof.

Embodiment P23: The method of any one of embodiments P1-P17, wherein the promoter comprises a promoter having the nucleotide sequence of SEQ ID NO: 12, or a fragment thereof.

Embodiment P24: The method of any one of embodiments P1-P17, wherein the promoter comprises a promoter having the nucleotide sequence of SEQ ID NO: 14, or a fragment thereof.

Embodiment P25: The method of any one of embodiments P1-P17, wherein the promoter comprises a promoter having the nucleotide sequence of SEQ ID NO: 16, or a fragment thereof.

Embodiment P26: The method of any one of embodiments P1-P17, wherein the promoter comprises a promoter having the nucleotide sequence of SEQ ID NO: 18, or a fragment thereof.

Embodiment P27: The method of any one of embodiments P1-P17, wherein the promoter comprises a promoter having the nucleotide sequence of SEQ ID NO: 20, or a fragment thereof.

Embodiment P28: The method of any one of embodiments P1-P17, wherein the promoter comprises a promoter having the nucleotide sequence of SEQ ID NO: 31, or a fragment thereof.

Embodiment P29: The method of any one of embodiments P1-P17, wherein the promoter comprises a promoter having the nucleotide sequence of SEQ ID NO: 32, or a fragment thereof.

Embodiment P30: The method of any one of embodiments P1-P29, wherein the promoter comprises an additional viral intron.

Embodiment P31: The method of any one of embodiments P1-P30, wherein the additional viral intron comprises the nucleotide sequence of SEQ ID NO: 10, or a fragment thereof.

Embodiment P32: The method of any one of embodiments P1-P31, wherein the vector is an AAV2 vector.

Embodiment P33: The method of any one of embodiments P1-P32, wherein the vector comprises a CMV promoter.

Embodiment P34: The method of any one of embodiments P1-P33, wherein the vector comprises a Kozak sequence.

Embodiment P35: The method of any one of embodiments P1-P34, wherein the vector comprises one or more ITR sequence flanking the vector portion encoding CFH.

Embodiment P36: The method of any one of embodiments P1-P35, wherein the vector comprises a polyadenylation sequence.

Embodiment P37: The method of any one of embodiments P1-P35, wherein the vector comprises a selective marker.

Embodiment P38: The method of embodiment P37, wherein the selective marker is an antibiotic-resistance gene.

Embodiment P39: The method of embodiment P38, wherein the antibiotic-resistance gene is an ampicillin-resistance gene.

Embodiment P40: The method of any one of embodiments P1-P39, wherein the vector or composition is capable of inducing at least 20%, 50%, 100%, 150%, 200%, 250%, 300%, 400%, 500%, 700%, 900%, 1000%, 1100%, 1500%, or 2000% expression of CFH polypeptide or biologically active fragment or variant thereof in a target cell (e.g., an RPE or liver cell) as compared to the endogenous expression of CFH in the target cell.

Embodiment P41: The method of any one of embodiments P1-P39, wherein the expression of the vector or composition in a target cell (e.g., an RPE or liver cell) results in at least 20%, 50%, 100%, 150%, 200%, 250%, 300%, 400%, 500%, 700%, 900%, 1000%, 1100%, 1500%, or 2000% levels of CFH polypeptide or biologically active fragment or variant thereof activity in the target cell as compared to endogenous levels of CFH activity in the target cell.

Embodiment P42: The method of any one of embodiments P1-P41, wherein the vector or composition induces CFH polypeptide or biologically active fragment or variant thereof expression in a target cell of the eye.

Embodiment P43: The method of embodiment P42, wherein the vector or composition induces CFH polypeptide or biologically active fragment or variant thereof expression in a target cell of the retina or macula.

Embodiment P44: The method of embodiment P42 or P43, wherein the target cell of the retina is selected from the group of layers consisting of: inner limiting membrane, nerve fiber, ganglion cell layer (GCL), inner plexiform layer, inner nuclear layer, outer plexiform layer, outer nuclear layer, external limiting membrane, rods and cones, and retinal pigment epithelium (RPE).

Embodiment P45: The vector or composition of embodiment P43, wherein the target cell is in the choroid plexus.

Embodiment P46: The vector or composition of embodiment P43, wherein the target cell is in the macula.

Embodiment P47: The method of any one of embodiments P1-P46, wherein the vector induces CFH expression in a cell of the GCL and/or RPE.

Embodiment P48: The AAV vector of any one of embodiments P1-P47, wherein the vector comprises a promoter having the nucleotide sequence of SEQ ID NO: 36, or a fragment thereof.

Embodiment P49: The method of any one of embodiments P1-P48, wherein the vector comprises a promoter that is associated with strong expression in the liver.

Embodiment P50: The method of embodiment P49, wherein the promoter comprises a nucleotide sequence that is at least 90%, 95% or 100% identical to the nucleotide sequence of any one of SEQ ID NOs: 16, 18, or 20.

Embodiment P51: The method of any one of embodiments P1-P48, wherein the vector comprises a promoter that is associated with strong expression in the eye.

Embodiment P52: The method of embodiment P51, wherein the promoter comprises a nucleotide sequence that is at least 90%, 95%, or 100% identical to the nucleotide sequence of any one of SEQ ID NOs: 6 or 32.

Embodiment P53: The method of any one of embodiments P1-P52, wherein the subject has a loss-of-function mutation in the subject's CFH gene.

Embodiment P54: The method of any one of embodiments P1-P53, wherein the subject has one or more CFH mutations selected from the group consisting of: R2T, L3V, R53C, R53H, S58A, D90G, D130N, R175Q, R175P, I221V, R303W, R303Q, Q400K, Y402H, P503A, R567G, G650V, S8901, T956M, G1194D, or R1210C.

Embodiment P55: The method of any one of embodiments P1-P53, wherein the subject has any one or more of the following CFH mutations: R2T, R53C, R53H, S58A, D130N, R175Q, R175P, 1221V, R303W, R303Q, P503A, R567G, G650V, G1194D, or R1210C.

Embodiment P56: The method of any one of embodiments P1-P53, wherein the subject has an R2T mutation.

Embodiment P57: The method of any one of embodiments P1-P53, wherein the subject has an L3V mutation.

Embodiment P58: The method of any one of embodiments P1-P53, wherein the subject has an R53C mutation.

Embodiment P59: The method of any one of embodiments P1-P53, wherein the subject has an R53H mutation.

Embodiment P60: The method of any one of embodiments P1-P53, wherein the subject has an S58A mutation.

Embodiment P61: The method of any one of embodiments P1-P53, wherein the subject has a D90G mutation.

Embodiment P62: The method of any one of embodiments P1-P53, wherein the subject has a D130N mutation.

Embodiment P63: The method of any one of embodiments P1-P53, wherein the subject has an R175Q mutation.

Embodiment P64: The method of any one of embodiments P1-P53, wherein the subject has an R175P mutation.

Embodiment P65: The method of any one of embodiments P1-P53, wherein the subject has an I221V mutation.

Embodiment P66: The method of any one of embodiments P1-P53, wherein the subject has an R303W mutation.

Embodiment P67: The method of any one of embodiments P1-P53, wherein the subject has an R303Q mutation.

Embodiment P68: The method of any one of embodiments P1-P53, wherein the subject has a Q400K mutation.

Embodiment P69: The method of any one of embodiments P1-P53, wherein the subject has a Y402H mutation.

Embodiment P70: The method of any one of embodiments P1-P53, wherein the subject has a P503A mutation.

Embodiment P71: The method of any one of embodiments P1-P53, wherein the subject has an R567G mutation.

Embodiment P72: The method of any one of embodiments P1-P53, wherein the subject has a G650V mutation.

Embodiment P73: The method of any one of embodiments P1-P53, wherein the subject has an S890I mutation.

Embodiment P74: The method of any one of embodiments P1-P53, wherein the subject has a T956M mutation.

Embodiment P75: The method of any one of embodiments P1-P53, wherein the subject has a G1194D mutation.

Embodiment P76: The method of any one of embodiments P1-P53, wherein the subject has an R1210C mutation.

Embodiment P77: The method of any one of embodiments P1-P76, wherein the subject expresses a mutant CFH polypeptide having reduced CFH activity as compared to a wildtype CFH polypeptide (e.g., a CFH polypeptide having the amino acid sequence of any one of SEQ ID NOs: 33, 34, 37 or 38).

Embodiment P78: The method of embodiment P77, wherein the CFH activity is the ability to bind to C3b.

Embodiment P79: The method of embodiment P77, wherein the CFH activity has the ability to act as a cofactor with CFI and facilitate C3b cleavage.

Embodiment P80: The method of embodiment P77, wherein the CFH activity is the ability to bind to a cell surface (e.g., an erythrocyte and/or endothelial cell).

Embodiment P81: The method of embodiment P77, wherein the CFH activity is the ability to bind to heparin.

Embodiment P82: The method of embodiment P77, wherein the CFH activity is the ability to reduce C5b9 levels generated as a result of complement activation, e.g., as measured in a Wieslab AP assay.

Embodiment P83: The method of embodiment P77, wherein the CFH activity is the ability to inhibit hemolysis.

Embodiment P84: The method of any one of embodiments P1-P83, wherein if a CFH polypeptide having the CFH mutation were tested in a functional assay, the mutant CFH polypeptide would display reduced CFH activity as compared to a wildtype CFH polypeptide (e.g., a CFH polypeptide having the amino acid sequence of any one of SEQ ID NOs: 33, 34, 37 or 38).

Embodiment P85: The method of embodiment P84, wherein the functional assay tests the ability of CFH to bind to C3b.

Embodiment P86: The method of embodiment P84, wherein the functional assay tests the ability of CFH to act as a cofactor with CFI and facilitate C3b cleavage.

Embodiment P87: The method of embodiment P84, wherein the functional assay tests the ability of CFH to bind to a cell surface (e.g., an erythrocyte and/or endothelial cell).

Embodiment P88: The method of embodiment P84, wherein the functional assay tests the ability of CFH to bind to heparin.

Embodiment P89: The method of embodiment P84, wherein the functional assay tests the ability of CFH to reduce C5b9 levels generated as a result of complement activation, e.g., as measured in a Wieslab AP assay.

Embodiment P90: The method of embodiment P84, wherein the functional assay tests the ability of CFH to inhibit hemolysis.

Embodiment P91: The method of any one of embodiments P1-P90, wherein the subject has atypical hemolytic uremic syndrome (aHUS).

Embodiment P92: The method of any one of embodiments P1-P91, wherein the subject is homozygous for at least one of the one or more CFH mutations.

Embodiment P93: The method of any one of embodiments P1-P91, wherein the subject is heterozygous for at least one of the one or more CFH mutations.

Embodiment P94: The method of any one of embodiments P1-P93, wherein the subject is suffering from a renal disease or complication.

Embodiment P95: The method of any one of embodiments P1-P94, wherein the vector or composition is administered to the retina at a dose in the range of 1×10¹⁰ vg/eye to 1×10¹³ vg/eye.

Embodiment P96: The method of embodiment P95, wherein the vector or composition is administered to the retina at a dose of about 1.4×10¹² vg/eye.

Embodiment P97: The method of any one of embodiments P1-P96, wherein the vector is administered intravitreally.

Embodiment P98: The method of any one of embodiments P1-P97, wherein the subject is a subject in whom it has been determined has one or more CFH mutations.

Embodiment P99: The method of embodiment P98, wherein the subject is a subject in whom it has been determined has any one of the following CFH mutations: R2T, L3V, R53C, R53H, S58A, D90G, D130N, R175Q, R175P, I221V, R303W, R303Q, Q400K, Y402H, P503A, R567G, G650V, S8901, T956M, G1194D, or R1210C.

Embodiment P100: The method of embodiment P98, wherein the subject is a subject in whom it has been determined has any one or more of the following CFH mutations: R2T, R53C, R53H, S58A, D130N, R175Q, R175P, I221V, R303W, R303Q, P503A, R567G, G650V, G1194D, or R1210C.

Embodiment P101: The method of embodiment P98, wherein the subject is a subject in whom it has been determined has an R2T mutation.

Embodiment P102: The method of embodiment P98, wherein the subject is a subject in whom it has been determined has an L3V mutation.

Embodiment P103: The method of embodiment P98, wherein the subject is a subject in whom it has been determined has an R53C mutation.

Embodiment P104: The method of embodiment P98, wherein the subject is a subject in whom it has been determined has an R53H mutation.

Embodiment P105: The method of embodiment P98, wherein the subject is a subject in whom it has been determined has an S58A mutation.

Embodiment P106: The method of embodiment P98, wherein the subject is a subject in whom it has been determined has a D90G mutation.

Embodiment P107: The method of embodiment P98, wherein the subject is a subject in whom it has been determined has a D130N mutation.

Embodiment P108: The method of embodiment P98, wherein the subject is a subject in whom it has been determined has an R175Q mutation.

Embodiment P109: The method of embodiment P98, wherein the subject is a subject in whom it has been determined has an R175P mutation.

Embodiment P110: The method of embodiment P98, wherein the subject is a subject in whom it has been determined has an I221V mutation.

Embodiment P111: The method of embodiment P98, wherein the subject is a subject in whom it has been determined has an R303W mutation.

Embodiment P112: The method of embodiment P98, wherein the subject is a subject in whom it has been determined has an R303Q mutation.

Embodiment P113: The method of embodiment P98, wherein the subject is a subject in whom it has been determined has a Q400K mutation.

Embodiment P114: The method of embodiment P98, wherein the subject is a subject in whom it has been determined has a Y402H mutation.

Embodiment P115: The method of embodiment P98, wherein the subject is a subject in whom it has been determined has a P503A mutation.

Embodiment P116: The method of embodiment P98, wherein the subject is a subject in whom it has been determined has an R567G mutation.

Embodiment P117: The method of embodiment P98, wherein the subject is a subject in whom it has been determined has a G650V mutation.

Embodiment P118: The method of embodiment P98, wherein the subject is a subject in whom it has been determined has an S890I mutation.

Embodiment P119: The method of embodiment P98, wherein the subject is a subject in whom it has been determined has a T956M mutation.

Embodiment P120: The method of embodiment P98, wherein the subject is a subject in whom it has been determined has a G1194D mutation.

Embodiment P121: The method of embodiment P98, wherein the subject is a subject in whom it has been determined has a R1210C mutation.

Embodiment P122: The method of any one of embodiments P98-P121, wherein the subject is a subject in whom it has been determined is homozygous for at least one of the one or more CFH mutations.

Embodiment P123: The method of any one of embodiments P98-P121, wherein the subject is a subject in whom it has been determined is heterozygous for at least one of the one or more CFH mutations.

Embodiment P124: The method of any one of embodiments P1-P123, wherein the vector is an AAV.7m8 vector.

Embodiment P125: The method of any one of embodiments P1-P124, wherein the subject is homozygous for a Y402H mutation.

Embodiment P126: The method of any one of embodiments P1-P125, wherein the subject is a subject in whom it has been determined is homozygous for a Y402H mutation.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the disclosure should be determined by reference to the claims, along with their full scope of equivalents, and the specification, along with such variations.

1. A method of treating a subject having a disease or disorder associated with undesired activity of the alternative complement pathway, comprising the step of administering to the subject an adeno-associated viral (AAV) vector comprising a nucleotide sequence encoding a Complement Factor H (CFH) polypeptide or biologically active fragment or variant thereof, and wherein the subject has one or more CFH, Complement Component 3 (C3), and/or Complement Factor B (CFB) gene mutations.

2. A method of treating a subject having age-related macular degeneration (AMD), comprising the step of administering to the subject an adeno-associated viral (AAV) vector comprising a nucleotide sequence encoding a Complement Factor H (CFH) polypeptide or biologically active fragment or variant thereof, and wherein the subject has one or more CFH, C3, and/or CFB gene mutations.

3. The method of claim 1 or 2, wherein the amino acid sequence of the CFH polypeptide is at least 90% identical to the amino acid sequence of SEQ ID NO: 33 or a fragment thereof.

4. The method of any one of claims 1-3, wherein the amino acid sequence of the CFH polypeptide is at least 95% identical to the amino acid sequence of SEQ ID NO: 33 or a fragment thereof.

5. The method of any one of claims 1-4, wherein the amino acid sequence of the CFH polypeptide comprises the amino acid sequence of SEQ ID NO: 33 or a fragment thereof.

6. The method of any one of claims 1-5, wherein the nucleotide sequence is at least 90% identical to the nucleotide sequence of SEQ ID NO: 1, 2, 3 or 5, or codon-optimized variant and/or a fragment thereof.

7. The method of any one of claims 1-6, wherein the nucleotide sequence is the sequence of SEQ ID NO: 1, 2, 3 or 5, or codon-optimized variant and/or a fragment thereof.

8. The method of any one of claims 1-7, wherein the CFH polypeptide or biologically active fragment or variant thereof comprises the V62 polymorphism.

9. The method of any one of claims 1-8, wherein the CFH polypeptide or biologically active fragment or variant thereof comprises the Y402 polymorphism.

10. The method of any one of claims 1-9, wherein the CFH polypeptide or biologically active fragment or variant thereof comprises at least four CCP domains.

11. The method of any one of claims 1-10, wherein the CFH polypeptide or biologically active fragment or variant thereof comprises at least five CCP domains.

12. The method of any one of claims 1-11, wherein the CFH polypeptide or biologically active fragment or variant thereof comprises at least six CCP domains.

13. The method of any one of claims 1-12, wherein the CFH polypeptide or biologically active fragment or variant thereof comprises at least seven CCP domains.

14. The method of any one of claims 1-9, wherein the CFH polypeptide or biologically active fragment or variant thereof comprises at least three CCP domains.

15. The method of any one of claims 1-14, wherein the CFH polypeptide or biologically active fragment or variant thereof comprises the amino acid sequence of SEQ ID NO: 4.

16. The method of claim 15, wherein the amino acid sequence of SEQ ID NO: 4 is the C-terminal sequence of the CFH polypeptide or biologically active fragment or variant thereof.

17. The method of any one of claims 1-16, wherein the CFH polypeptide or biologically active fragment or variant thereof is capable of diffusing across the Bruch's membrane.

18. The method of any one of claims 1-17, wherein the CFH polypeptide or biologically active fragment or variant thereof is capable of binding C3b.

19. The method of any one of claims 1-18, wherein the CFH polypeptide or biologically active fragment or variant thereof is capable of facilitating the breakdown of C3b.

20. The method of any one of claims 1-19, wherein the vector comprises a promoter operably linked to the nucleotide sequence encoding the CFH polypeptide or biologically active fragment or variant thereof.

21. The method of claim 20, wherein the promoter is less than 1000 nucleotides in length.

22. The method of claim 20 or 21, wherein the promoter is less than 500 nucleotides in length.

23. The method of any one of claims 20-22, wherein the promoter is less than 400 nucleotides in length.

24. The method of any one of claims 20-23, wherein the promoter comprises a CMV promoter.

25. The method of any one of claims 20-23, wherein the promoter comprises the nucleotide sequence of SEQ ID NO: 8, or a fragment thereof.

26. The method of any one of claims 20-23, wherein the promoter comprises the nucleotide sequence of SEQ ID NO: 12, or a fragment thereof.

27. The method of any one of claims 20-23, wherein the promoter comprises the nucleotide sequence of SEQ ID NO: 14, or a fragment thereof.

28. The method of any one of claims 20-23, wherein the promoter comprises the nucleotide sequence of SEQ ID NO: 31, or a fragment thereof.

29. The method of any one of claims 20-23, wherein the promoter comprises the nucleotide sequence of SEQ ID NO: 36, or a fragment thereof.

30. The method of any one of claims 20-23, wherein the promoter is associated with strong expression in the eye.

31. The method of claim 30, wherein the promoter comprises a nucleotide sequence at least 90%, 95%, or 100% identical to the nucleotide sequence of any one of SEQ ID NOs: 6 or 32.

32. The method of claim 30 or 31, wherein the promoter comprises the nucleotide sequence of SEQ ID NO: 6, or a fragment thereof.

33. The method of claim 30 or 31, wherein the promoter comprises the nucleotide sequence of SEQ ID NO: 32, or a fragment thereof.

34. The method of any one of claims 20-23, wherein the promoter is associated with strong expression in the liver.

35. The method of claim 34, wherein the promoter comprises a nucleotide sequence at least 90%, 95% or 100% identical to the nucleotide sequence of any one of SEQ ID NOs: 16, 18, or 20.

36. The method of claim 34 or 35, wherein the promoter comprises the nucleotide sequence of SEQ ID NO: 16, or a fragment thereof.

37. The method of claim 34 or 35, wherein the promoter comprises the nucleotide sequence of SEQ ID NO: 18, or a fragment thereof.

38. The method of claim 34 or 35, wherein the promoter comprises the nucleotide sequence of SEQ ID NO: 20, or a fragment thereof.

39. The method of any one of claims 1-38, wherein the vector comprises a viral intron operably linked to the promoter.

40. The method of claim 39, wherein the viral intron comprises the nucleotide sequence of SEQ ID NO: 10, or a fragment thereof.

41. The method of any one of claims 1-40, wherein the vector comprises a Kozak sequence operably linked to the CFH polypeptide or biologically active fragment or variant thereof.

42. The method of any one of claims 1-41, wherein the vector comprises a polyadenylation sequence operably linked to the CFH polypeptide or biologically active fragment or variant thereof.

43. The method of any one of claims 1-42, wherein the vector comprises AAV2 capsid proteins.

44. The method of any one of claims 1-43, wherein the vector comprises AAV.7m8 capsid proteins.

45. The method of any one of claims 1-44, wherein the vector comprises one or more ITR sequence flanking the vector portion encoding CFH.

46. The method of any one of claims 1-45, wherein the vector comprises a selective marker.

47. The method of claim 46, wherein the selective marker is an antibiotic-resistance gene.

48. The method of claim 47, wherein the antibiotic-resistance gene is an ampicillin-resistance gene.

49. The method of any one of claims 1-48, wherein expression of the CFH polypeptide or biologically active fragment or variant thereof is induced in a target cell of the subject.

50. The method of claim 49, wherein the target cell is a cell of the eye.

51. The method of claim 50, wherein the target cell is a cell of the retina.

52. The method of claim 51, wherein target cell is a cell of a layer of the retina selected from the group consisting of inner limiting membrane, nerve fiber, ganglion cell layer (GCL), inner plexiform layer, inner nuclear layer, outer plexiform layer, outer nuclear layer, external limiting membrane, rods and cones, and retinal pigment epithelium (RPE).

53. The method of claim 52, wherein the target cell is an RPE cell.

54. The method of claim 50, wherein the target cell is a cell of the macula.

55. The method of any one of claims 1-49, wherein the vector is administered intraocularly.

56. The method of claim 55, wherein the vector is administered intravitreally.

57. The method of claim 55 or 56, wherein the vector is administered to the retina at a dose in the range of 1×10¹⁰ vg/eye to 1×10¹³ vg/eye.

58. The method of claim 57, wherein the vector or composition is administered to the retina at a dose of about 1.4×10¹² vg/eye.

59. The method of any one claims 50-58, wherein the level of the CFH polypeptide or biologically active fragment or variant thereof in the aqueous humor or vitreous humor of the subject is at least 50 ng/mL, at least 55 ng/mL, at least 60 ng/mL, at least 65 ng/mL, at least 70 ng/mL, at least 75 ng/mL, at least 80 ng/mL, at least 85 ng/mL, at least 90 ng/mL, or at least 100 ng/mL.

60. The method of claim 50, wherein the target cell is a liver cell.

61. The method of claim 60, wherein the vector is administered systemically.

62. The method of claim 61, wherein the vector is administered intravenously.

63. The method of any one of claims 60-62, wherein the level of the CFH polypeptide or biologically active fragment or variant thereof in the plasma of the subject is at least 100 μg/mL, at least 150 μg/mL, at least 200 μg/mL, at least 250 μg/mL, or at least 300 μg/mL.

64. The method of claim 50, wherein the target cell is a cell of the choroid plexus.

65. The method of any one of claims 1-64, wherein the subject has a mutation in the CFH gene, optionally wherein the mutation is a loss-of-function mutation.

66. The method of any one of claims 1-65, wherein the subject has one or more CFH mutations selected from the group consisting of: Y402H, R2T, L3V, R53C, R53H, S58A, D90G, D130N, R175Q, R175P, I221V, R303W, R303Q, Q400K, P503A, R567G, G650V, S8901, T956M, G1194D, or R1210C.

67. The method of any one of claims 1-65, wherein the subject has any one or more of the following CFH mutations: R2T, R53C, R53H, S58A, D130N, R175Q, R175P, 1221V, R303W, R303Q, P503A, R567G, G650V, G1194D, or R1210C.

68. The method of claim 65 or 66, wherein the subject has a Y402H mutation.

69. The method of claim 67, wherein the subject is homozygous for a Y402H mutation.

70. The method of any one of claims 65-67, wherein the subject has an R2T mutation.

71. The method of claim 65 or 66, wherein the subject has an L3V mutation.

72. The method of any one of claims 65-67, wherein the subject has an R53C mutation.

73. The method of any one of claims 65-67, wherein the subject has an R53H mutation.

74. The method of any one of claims 65-67, wherein the subject has an S58A mutation.

75. The method of claim 65 or 66, wherein the subject has a D90G mutation.

76. The method of any one of claims 65-67, wherein the subject has a D130N mutation.

77. The method of any one of claims 65-67, wherein the subject has an R175Q mutation.

78. The method of any one of claims 65-67, wherein the subject has an R175P mutation.

79. The method of any one of claims 65-67, wherein the subject has an 1221V mutation.

80. The method of any one of claims 65-67, wherein the subject has an R303W mutation.

81. The method of any one of claims 65-67, wherein the subject has an R303Q mutation.

82. The method of claim 65 or 66, wherein the subject has a Q400K mutation.

83. The method of any one of claims 65-67, wherein the subject has a P503A mutation.

84. The method of any one of claims 65-67, wherein the subject has an R567G mutation.

85. The method of any one of claims 65-67, wherein the subject has a G650V mutation.

86. The method of claim 65 or 66, wherein the subject has an S890I mutation.

87. The method of claim 65 or 66, wherein the subject has a T956M mutation.

88. The method of any one of claims 65-67, wherein the subject has a G1194D mutation.

89. The method of any one of claims 65-67, wherein the subject has an R1210C mutation.

90. The method of any one of claims 65-89, wherein the one or more CFH mutations reduce CFH activity as compared to a wildtype CFH polypeptide.

91. The method of claim 90, wherein the CFH activity is the ability to bind to C3b.

92. The method of claim 90, wherein the CFH activity has the ability to act as a cofactor with CFI and facilitate C3b cleavage.

93. The method of claim 90, wherein the CFH activity is the ability to bind to a cell surface.

94. The method of claim 90, wherein the CFH activity is the ability to bind to heparin.

95. The method of claim 90, wherein the CFH activity is the ability to reduce C5b9 levels generated as a result of complement activation.

96. The method of claim 90, wherein the CFH activity is the ability to inhibit hemolysis.

97. The method of any one of claims 90-96, wherein the wildtype CFH polypeptide comprises a CFH polypeptide having the amino acid sequence of any one of SEQ ID NOs: 33, 34, 37 or 38.

98. The method of any one of claims 1-97, wherein the subject has a mutation in the subject's C3 gene, optionally wherein the mutation is a gain-of-function mutation.

99. The method of claim 98, wherein the subject has one or more of the following C3 mutations: R102G, K155Q, V619M, and R735W.

100. The method of any one of claims 1-99, wherein the subject has a mutation in the subject's CFB gene, optionally wherein the mutation is a gain-of-function mutation.

101. The method of claim 100, wherein the subject has the I242L mutation of CFB.

102. The method of any one of claims 1-101, wherein the subject is homozygous for CFH 62V, C3 102G, and complement factor B (CFB) 32R.

103. The method of any one of claims 1-102, wherein the subject is homozygous for at least one of the one or more CFH, C3, and/or CFB mutations.

104. The method of any one of claims 1-103, wherein the subject is heterozygous for at least one of the one or more CFH, C3, and/or CFB mutations.

105. The method of any one of claims 1-104, wherein the subject has been determined to have the one or more CFH, C3, and/or CFB mutations.

106. The method of any one of claims 1-105, wherein the subject has atypical hemolytic uremic syndrome (aHUS).

107. The method of any one of claims 1-106, wherein the subject has a renal disease or complication.


SEQUENCE LISTING (43 sequences)

SEQ ID NO: 1 - DNA, 1381 bp, Artificial Sequence
  SEQ ID NO: 1 Codon Optimized Human Factor H Like 1 (FHL1)
gcggccgccaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctaataagcttggatccagatct

SEQ ID NO: 2 - DNA, 1381 bp, Artificial Sequence
  SEQ ID NO: 2-Codon Optimized Human Factor H Like 1 (FHL1)
gcggccgccaccatgagactgctggctaaaattatctgcctgatgctgtgggctatctgcgtcgctgaggattgtaacgagctgcccccccggagaaatacagagatcctgaccggctcttggagcgaccagacatatcccgagggcacccaggccatctacaagtgcaggcctggctatcgctctctgggcaacgtgatcatggtgtgcaggaagggagagtgggtggccctgaatcccctgaggaagtgccagaagcgcccttgtggacacccaggcgacacacccttcggcacctttacactgaccggcggcaacgtgttcgagtacggcgtgaaggccgtgtatacctgcaacgagggctaccagctgctgggcgagatcaattacagagagtgtgacacagatggctggaccaacgatatccctatctgcgaggtggtgaagtgtctgcctgtgaccgccccagagaatggcaagatcgtgagctccgccatggagccagacagggagtatcacttcggccaggccgtgcgcttcgtgtgcaactccggctacaagatcgagggcgatgaggagatgcactgtagcgacgatggcttctggtccaaggagaagcccaagtgcgtggagatcagctgtaagtcccctgacgtgatcaatggctctccaatcagccagaagatcatctataaggagaacgagaggtttcagtacaagtgcaatatgggctacgagtattctgagaggggcgatgccgtgtgcacagagagcggatggcggcccctgccttcctgcgaggagaagtcttgtgacaacccttatatcccaaatggcgattacagcccactgcggatcaagcacagaacaggcgatgagatcacctatcagtgccggaacggcttttaccccgccacaagaggcaataccgccaagtgtacatccaccggatggatcccagcaccaagatgcaccctgaagccctgtgactatcctgatatcaagcacggcggcctgtatcacgagaacatgagacggccctacttccctgtggccgtgggcaagtactattcctactattgcgacgagcactttgagacaccctccggctcttactgggaccacatccactgtacccaggatggatggagccccgcagtgccatgcctgaggaagtgttacttcccttatctggagaatggctacaaccagaattatggccgcaagtttgtgcagggcaagagcatcgatgtggcatgccacccaggatacgcactgccaaaggcacagaccacagtgacctgtatggaaaacggctggtcccctacccctcgctgtatcagagtgtcattcaccctgtaataagcttggatccagatct

SEQ ID NO: 3 - DNA, 1381 bp, Artificial Sequence
  SEQ ID NO: 3-Non-Codon Optimized Human Factor H Like 1 (FHL1)
gcggccgccaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatcgaagtcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctaataagcttggatccagatct

SEQ ID NO: 4 - protein, 4 aa, Artificial Sequence
  SEQ ID NO: 4: SFTL Sequence
SFTL

SEQ ID NO: 5 - DNA, 3696 bp, Artificial Sequence
  SEQ ID NO: 5-CFH Nucleotide Sequence
atgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatag

SEQ ID NO: 6 - DNA, 432 bp, Artificial Sequence
  SEQ ID NO: 6-CRALBP Promoter
acgcgttaactagtaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgaccatcgat

SEQ ID NO: 7 - DNA, 7296 bp, Artificial Sequence
  SEQ ID NO: 7-Representative CFH AAV vector (with CRALBP Promoter)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttaactagtaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgaccatcgatagctgcaggcggccgccgccaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaactcgagggatcccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 8 - DNA, 226 bp, Artificial Sequence
  SEQ ID NO: 8-EF1a Promoter
acgcgttaactagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 9 - DNA, 7096 bp, Artificial Sequence
  SEQ ID NO: 9-Representative CFH AAV vector (EF1a promoter)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttaactagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagatcgatagctgcaggcggccgccgccaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaactcgagggatcccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 10 - DNA, 150 bp, Artificial Sequence
  SEQ ID NO: 10-SV40i Intron
gtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgg

SEQ ID NO: 11 - DNA, 7246 bp, Artificial Sequence
  SEQ ID NO: 11-Representative CFH AAV vector (with EF1a promoter and SV40i intron)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttaactagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggatcgatagctgcaggcggccgccgccaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaactcgagggatcccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 12 - DNA, 476 bp, Artificial Sequence
  SEQ ID NO: 12-HSP70 Promoter
actagtcctgcagggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaacc

SEQ ID NO: 13 - DNA, 7344 bp, Artificial Sequence
  SEQ ID NO: 13-Representative CFH AAV Vector (with HSP70 Promoter)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgttaactagtcctgcagggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccctgcagatcgatgcggccgcaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaactcgagggatcccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 14 - DNA, 272 bp, Artificial Sequence
  SEQ ID NO: 14-sCBA Promoter
actagtccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggg

SEQ ID NO: 15 - DNA, 7300 bp, Artificial Sequence
  SEQ ID NO: 15-Representative CFH AAV Vector (with sCBA Promoter and SV40i Intron)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttaactagtccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggatcgatagctgcaggcggccgccgccaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaactcgagggatcccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 16 - DNA, 353 bp, Artificial Sequence
  SEQ ID NO: 16-hAAT1 Promoter
gttaacggctgcccactgggcatttcataggtggctcagtcctcttccctctgcagctggccccagaaacctgccagttattggtgccaggtctgtgccaggagggcgaggcctgtcatttctagtaatcctctgggcagtgtgactgtacctcttgcggcaactcaaagggagagggtgacttgtcccgggtcacagagctgaaagggcaggtacaacaggtgacatgccgggctgtctgagtttatgagggcccagtcttgtgtctgccgggcaatgagcaaggctccttcctgtccaagctccccgcccctccccagcctactgcctccacccgaagtctacttcctggg

SEQ ID NO: 17 - DNA, 7229 bp, Artificial Sequence
  SEQ ID NO: 17-Representative CFH AAV Vector (with hAAT1 promoter)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttaacggctgcccactgggcatttcataggtggctcagtcctcttccctctgcagctggccccagaaacctgccagttattggtgccaggtctgtgccaggagggcgaggcctgtcatttctagtaatcctctgggcagtgtgactgtacctcttgcggcaactcaaagggagagggtgacttgtcccgggtcacagagctgaaagggcaggtacaacaggtgacatgccgggctgtctgagtttatgagggcccagtcttgtgtctgccgggcaatgagcaaggctccttcctgtccaagctccccgcccctccccagcctactgcctccacccgaagtctacttcctgggatcgatagctgcaggcggccgccgccaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaactcgagggatcccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 18 - DNA, 159 bp, Artificial Sequence
  SEQ ID NO: 18-ALB Promoter
gttaacacgcgttaactagtcagttccagatggtaaatatacacaagggatttagtcaaacaattttttggcaagaatattatgaattttgtaatcggttggcagccaatgaaatacaaagatgagtctagttaataatctacaattattggttaaaga

SEQ ID NO: 19 - DNA, 7035 bp, Artificial Sequence
  SEQ ID NO: 19-Representative CFH AAV Vector (with ALB Promoter)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttaacacgcgttaactagtcagttccagatggtaaatatacacaagggatttagtcaaacaattttttggcaagaatattatgaattttgtaatcggttggcagccaatgaaatacaaagatgagtctagttaataatctacaattattggttaaagaatcgatagctgcaggcggccgccgccaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaactcgagggatcccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 20 - DNA, 496 bp, Artificial Sequence
  SEQ ID NO: 20-PCK1 Promoter
gttaacagcccccagttaggttaggcatttccaatctttgccaataagccacatatttgcccaagttagggtgcatccttcccatgaactttgactgtgacctttgactatggggtgacatcttatagctgtggtgttttgccaaccagcagctcttggtacacaaaatgtgctgctagcaggtgccccggccaaccttgtccttgacccacctgcctgttaagaaaagggtgttgtgttttgcaacagcagtaaaatgggtcaaggtttagtcagttggaagttgtgtcaaaactcactatggttggttgagggctcgaagtctcccagcattcattaacaactatctgttcaatgattatctccctggggcgtgttgcagtgagttggcccaaagcataactgaccctggccgtgatccagagacctgccccctgacgtcagtggcgagcctccctgggtgcagctgaggggcagggctattcttttccacagt

SEQ ID NO: 21 - DNA, 7372 bp, Artificial Sequence
  SEQ ID NO: 21-Representative CFH AAV Vector (with PCK1Promoter)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttaacagcccccagttaggttaggcatttccaatctttgccaataagccacatatttgcccaagttagggtgcatccttcccatgaactttgactgtgacctttgactatggggtgacatcttatagctgtggtgttttgccaaccagcagctcttggtacacaaaatgtgctgctagcaggtgccccggccaaccttgtccttgacccacctgcctgttaagaaaagggtgttgtgttttgcaacagcagtaaaatgggtcaaggtttagtcagttggaagttgtgtcaaaactcactatggttggttgagggctcgaagtctcccagcattcattaacaactatctgttcaatgattatctccctggggcgtgttgcagtgagttggcccaaagcataactgaccctggccgtgatccagagacctgccccctgacgtcagtggcgagcctccctgggtgcagctgaggggcagggctattcttttccacagtatcgatagctgcaggcggccgccgccaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaattatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggttaactcgagggatcccggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 22 - DNA, 4681 bp, Artificial Sequence
  SEQ ID NO: 22-Representative FHL AAV Vector (with EF1a Promoter)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccggggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagaccggtctcgaaggcctgcaggcggccgccgccaccatgaatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctgataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 23 - DNA, 4607 bp, Artificial Sequence
  SEQ ID NO: 23-Representative FHL AAV Vector (with ALB Promoter)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagttccagatggtaaatatacacaagggatttagtcaaacaattttttggcaagaatattatgaattttgtaatcggttggcagccaatgaaatacaaagatgagtctagttaataatctacaattattggttaaagaccggtctcgaaggcctgcaggcggccgccgccaccatgaatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctgataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 24 - DNA, 4607 bp, Artificial Sequence; identical to SEQ ID NO: 23
  SEQ ID NO: 24-Representative FHL AAV Vector (with AAT1 Promoter)

SEQ ID NO: 25 - DNA, 4845 bp, Artificial Sequence
  SEQ ID NO: 25-Representative FHL AAV Vector (with EF1a.SV40i Promoter)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgacgcgttaactagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggaccggtctcgaaggcctgcaggcggccgccgccaccatgaatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctgataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 26 - DNA, 5441 bp, Artificial Sequence
  SEQ ID NO: 26-Representative FHL AAV Vector (with CAG Promoter)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgttggcaaagaattctgcagtcgacggtaccgcgggcccgggatccaccggtcgccaccatggtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctgtagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcaccggtctcgaaggcctgcaggcggccgccgccaccatgaatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctgataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 27 - DNA, 4895 bp, Artificial Sequence
  SEQ ID NO: 27-Representative FHL AAV Vector (with CRALBP Promoter)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgacgcgttaactagtaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgaccaccggtctcgaaggcctgcaggcggccgccgccaccatgaatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctgataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 28 - DNA, 6045 bp, Artificial Sequence
  SEQ ID NO: 28-Representative FHL AAV Vector (with hRPE65 Promoter)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgtatatttattgaagtttaatattgtgtttgtgatacagaagtatttgctttaattctaaataaaaattttatgcttttattgctggtttaagaagatttggattatccttgtactttgaggagaagtttcttatttgaaatattttggaaacaggtcttttaatgtggaaagatagatattaatctcctcttctattactctccaagatccaacaaaagtgattataccccccaaaatatgatggtagtatcttatactaccatcattttataggcatagggctcttagctgcaaataatggaactaactctaataaagcagaacgcaaatattgtaaatattagagagctaacaatctctgggatggctaaaggatggagcttggaggctacccagccagtaacaatattccgggctccactgttgaatggagacactacaactgccttggatgggcagagatattatggatgctaagccccaggtgctaccattaggacttctaccactgtccctaacgggtggagcccatcacatgcctatgccctcactgtaaggaaatgaagctactgttgtatatcttgggaagcacttggattaattgttatacagttttgttgaagaagacccctagggtaagtagccataactgcacactaaatttaaaattgttaatgagtttctcaaaaaaaatgttaaggttgttagctggtatagtatatatcttgcctgttttccaaggacttctttgggcagtaccttgtctgtgctggcaagcaactgagacttaatgaaagagtattggagatatgaatgaattgatgctgtatactctcagagtgccaaacatataccaatggacaagaaggtgaggcagagagcagacaggcattagtgacaagcaaagatatgcagaatttcattctcagcaaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatgaaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaagccataactccttttaagggatttagaaggcataaaaaggcccctggctgagaacttccttcttcattctgcagtaccggtctcgaaggcctgcaggcggccgccgccaccatgaatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctgataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 29 - DNA, 4948 bp, Artificial Sequence
  SEQ ID NO: 29-Representative FHL AAV Vector (with HSP70 Promoter)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgttaactagtcctgcagggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccaccggtctcgaaggcctgcaggcggccgccgccaccatgaatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctgataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 30 - DNA, 4959 bp, Artificial Sequence
  SEQ ID NO: 30-Representative FHL AAV Vector (with PCK1 Promoter)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgagcccccagttaggttaggcatttccaatctttgccaataagccacatatttgcccaagttagggtgcatccttcccatgaactttgactgtgacctttgactatggggtgacatcttatagctgtggtgttttgccaaccagcagctcttggtacacaaaatgtgctgctagcaggtgccccggccaaccttgtccttgacccacctgcctgttaagaaaagggtgttgtgttttgcaacagcagtaaaatgggtcaaggtttagtcagttggaagttgtgtcaaaactcactatggttggttgagggctcgaagtctcccagcattcattaacaactatctgttcaatgattatctccctggggcgtgttgcagtgagttggcccaaagcataactgaccctggccgtgatccagagacctgccccctgacgtcagtggcgagcctccctgggtgcagctgaggggcagggctattcttttccacagtaccggtctcgaaggcctgcaggcggccgccgccaccatgaatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctgataagatatcgatacattgatgagtttggacaaaccacaactagaatgcagtgaaaaaaatgctttatttgtgaaatttgtgatgctattgctttatttgtaaccattataagctgcaataaacaagatatcgttaactcgagggatcccacgtgctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 31 - DNA, 978 bp, Artificial Sequence
  SEQ ID NO: 31-CAG Promoter
gttaacttggcaaagaattctgcagtcgacggtaccgcgggcccgggatccaccggtcgccaccatggtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctgtagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatc

SEQ ID NO: 32 - DNA, 1585 bp, Artificial Sequence
  SEQ ID NO: 32- hRPE65 Promoter
gttaactatatttattgaagtttaatattgtgtttgtgatacagaagtatttgctttaattctaaataaaaattttatgcttttattgctggtttaagaagatttggattatccttgtactttgaggagaagtttcttatttgaaatattttggaaacaggtcttttaatgtggaaagatagatattaatctcctcttctattactctccaagatccaacaaaagtgattataccccccaaaatatgatggtagtatcttatactaccatcattttataggcatagggctcttagctgcaaataatggaactaactctaataaagcagaacgcaaatattgtaaatattagagagctaacaatctctgggatggctaaaggatggagcttggaggctacccagccagtaacaatattccgggctccactgttgaatggagacactacaactgccttggatgggcagagatattatggatgctaagccccaggtgctaccattaggacttctaccactgtccctaacgggtggagcccatcacatgcctatgccctcactgtaaggaaatgaagctactgttgtatatcttgggaagcacttggattaattgttatacagttttgttgaagaagacccctagggtaagtagccataactgcacactaaatttaaaattgttaatgagtttctcaaaaaaaatgttaaggttgttagctggtatagtatatatcttgcctgttttccaaggacttctttgggcagtaccttgtctgtgctggcaagcaactgagacttaatgaaagagtattggagatatgaatgaattgatgctgtatactctcagagtgccaaacatataccaatggacaagaaggtgaggcagagagcagacaggcattagtgacaagcaaagatatgcagaatttcattctcagcaaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatgaaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaagccataactccttttaagggatttagaaggcataaaaaggcccctggctgagaacttccttcttcattctgcagttgg

SEQ ID NO: 33 - protein, 1230 aa, Artificial Sequence
  SEQ ID NO: 33- CFH Amino Acid Sequence
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAK

SEQ ID NO: 34 - protein, 449 aa, Artificial Sequence
  SEQ ID NO: 34- FHL1 Amino Acid Sequence
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 35 - skipped

SEQ ID NO: 36 - DNA, 223 bp, Artificial Sequence
  SEQ ID NO: 36- MECP Promoter Sequence
ggccgaaatggacaggaaatctcgccaattgacggcatcgccgctgagactcccccctcccccgtcctccccgtcccagcccggccatcacagccaatgacgggcgggctcgcagcggcgccgagggcggggcgcgggcgcgcaggtgcagcagcgcgcgggccggccaagagggcggggcgcgacgtcggccgtgcggggtcccggcgtcggcggcgcgcgc

SEQ ID NO: 37 - protein, 1213 aa, Artificial Sequence
  SEQ ID NO: 37- Exemplary CFH Amino Acid Sequence
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 38 - protein, 1231 aa, Artificial Sequence
  SEQ ID NO: 38- Exemplary Precursor CFH Amino Acid Sequence
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 39 - protein, 246 aa, Artificial Sequence
  SEQ ID NO: 39- CFH (1-4) Amino Acid Sequence
DCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEK

SEQ ID NO: 40 - protein, 304 aa, Artificial Sequence
  SEQ ID NO: 40- CFH (1-5) Amino Acid Sequence
DCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 41 - protein, 426 aa, Artificial Sequence
  SEQ ID NO: 41- CFH (1-7) Amino Acid Sequence
DCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRV

SEQ ID NO: 42 - protein, 126 aa, Artificial Sequence
  SEQ ID NO: 42- CFH (19-20) Amino Acid Sequence
TGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 43 - protein, 185 aa, Artificial Sequence
  SEQ ID NO: 43- CFH (18-20) Amino Acid Sequence
SCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR